(19) United States
(12) Patent Application Publication
(10) Pub. No.: US 2022/0056154 A1
(43) Pub. Date: Feb. 24, 2022

(54) Bispecific Antibody Compositions and Methods for Treating COVID-19

(71) Applicant: Maddon Advisors LLC, Scarsdale, NY (US)

(72) Inventor: Paul J. Maddon, Scarsdale, NY (US)

(73) Assignee: Maddon Advisors LLC, Scarsdale, NY (US)

(21) Appl. No.: 17/499,030

(22) Filed: Oct. 12, 2021

Related U.S. Application Data
(63) Continuation-in-part of application No. PCT/US21/26811, filed on Apr. 12, 2021.
(60) Provisional application No. 63/029,765, filed on May 26, 2020.
(60) Provisional application No. 63/029,772, filed on May 26, 2020.
(60) Provisional application No. 63/028,627, filed on May 22, 2020.
(60) Provisional application No. 63/128,639, filed on Dec. 21, 2020.
(60) Provisional application No. 63/017,159, filed on Apr. 29, 2020.
(60) Provisional application No. 63/008,988, filed on Apr. 13, 2020.

(51) Int. Cl. C07K 16/40; C12N 15/86; C07K 16/46; C07K 16/28

(52) U.S. Cl. CPC C07K 16/40; C12N 15/86; C07K 2317/31; C07K 16/2803; C07K 2317/24; C07K 16/468

(57) ABSTRACT

This invention provides a bispecific antibody that (i) specifically binds to the extracellular portion of human angiotensin converting enzyme 2 (hACE2); (ii) specifically binds to the extracellular portion of human TMPRSS2 (hTMPRSS2); (iii) does not significantly inhibit the ability of hACE2 to cleave angiotensin II and/or a synthetic MCA-based peptide; and (iv) specifically inhibits the entry into hACE2⁺/hTMPRSS2⁺ human cells of a pseudovirus bearing SARS-CoV-2 S protein. This invention also provides related pharmaceutical compositions, recombinant nucleic acid molecules, vectors, AAV particles, therapeutic and prophylactic methods, and kits.

This application is a continuation-in-part of PCT International Application No. PCT/US21/26811, filed Apr. 12, 2021, which claims the benefit of U.S. Provisional Application No. 63/008,988, filed Apr. 13, 2020; U.S. Provisional Application No. 63/017,159, filed Apr. 29, 2020; U.S. Provisional Application No. 63/028,627, filed May 22, 2020; U.S. Provisional Application No. 63/028,639, filed May 22, 2020; U.S. Provisional Application No. 63/029,765, filed May 26, 2020; and U.S. Provisional Application No. 63/029,772, filed May 26, 2020, the contents of all of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to bispecific antibodies that target both human ACE2 and TMPRSS2, as well as related engineered viruses. These antibodies and viruses are useful for therapeutically and prophylactically addressing SARS-CoV-2 infection.

### BACKGROUND OF THE INVENTION

Since the beginning of the COVID-19 outbreak, there has been—and continues to be—an intensive worldwide effort to develop effective anti-SARS-CoV-2 therapeutics and prophylactics. To date, this nascent effort has yielded a few effective vaccines, but little success otherwise. For at least this reason, there is an urgent need for an effective way to treat and prevent SARS-CoV-2 infection.

### SUMMARY OF THE INVENTION

This invention provides a bispecific antibody that (i) specifically binds to the extracellular portion of human angiotensin converting enzyme 2 (hACE2); (ii) specifically binds to the extracellular portion of human TMPRSS2 (hTMPRSS2); (iii) does not significantly inhibit the ability of hACE2 to cleave angiotensin II and/or a synthetic MCA-based peptide; and (iv) specifically inhibits the entry into hACE2⁺/hTMPRSS2⁺ human cells of a pseudovirus bearing SARS-CoV-2 S protein.

This invention also provides an isolated nucleic acid molecule encoding (a) the present bispecific antibody, if the bispecific antibody has only one chain; or (b) one or more chains of the present bispecific antibody, if the bispecific antibody has a plurality of chains. This invention further provides a recombinant vector comprising the present nucleic acid molecule operably linked to a promoter of RNA transcription.

This invention further provides a composition comprising (i) the present bispecific antibody, and (ii) a pharmaceutically acceptable carrier.

This invention provides a method for reducing the likelihood of a human subject's becoming infected with SARS-CoV-2 comprising administering to the subject a prophylactically effective amount of the present bispecific antibody. This invention also provides a method for treating a human subject who is infected with SARS-CoV-2 comprising administering to the subject a therapeutically effective amount of the present bispecific antibody.

This invention provides a recombinant AAV vector comprising a nucleic acid sequence encoding (a) the present bispecific antibody, if the bispecific antibody has only one chain, or (b) one or more chains of the present bispecific antibody, if the bispecific antibody has a plurality of chains. This invention also provides a recombinant AAV particle comprising the present recombinant AAV vector and an AAV capsid protein. This invention further provides a composition comprising (i) a plurality of the present AAV particles and (ii) a pharmaceutically acceptable carrier.

This invention provides a method for reducing the likelihood of a human subject's becoming infected with SARS-CoV-2 comprising administering to the subject a prophylactically effective number of the present AAV particles. This invention also provides a method for treating a human subject who is infected with SARS-CoV-2 comprising administering to the subject a therapeutically effective number of the present AAV particles.

This invention further provides a kit comprising, in separate compartments, (a) a diluent and (b) the present bispecific antibody either as a suspension or in lyophilized form. This invention still further provides a kit comprising, in separate compartments, (a) a diluent and (b) a suspension of a plurality of the present recombinant AAV particles.

### BRIEF DESCRIPTION OF THE FIGURES

FIG. 1

This figure sets forth the amino acid sequence of hACE2, as well as the nucleic acid sequence encoding it (Tipnis, et al.).

FIG. 2

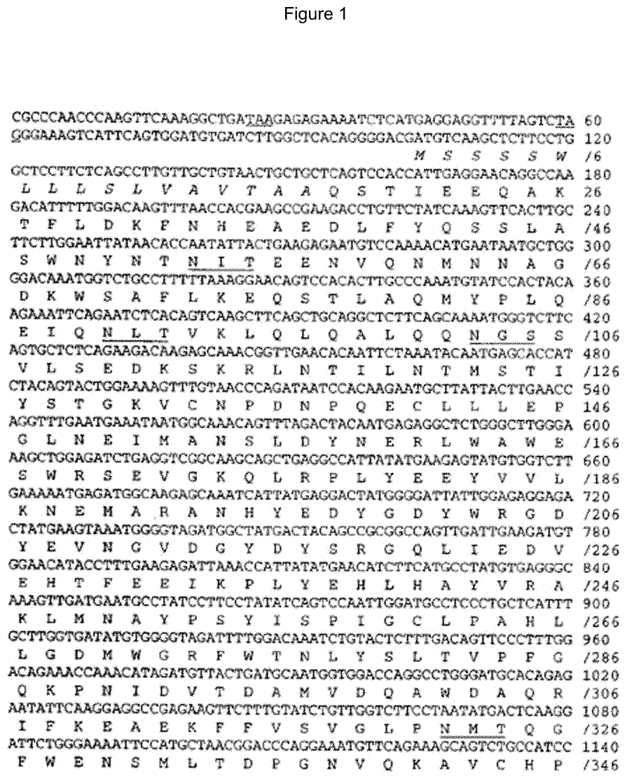

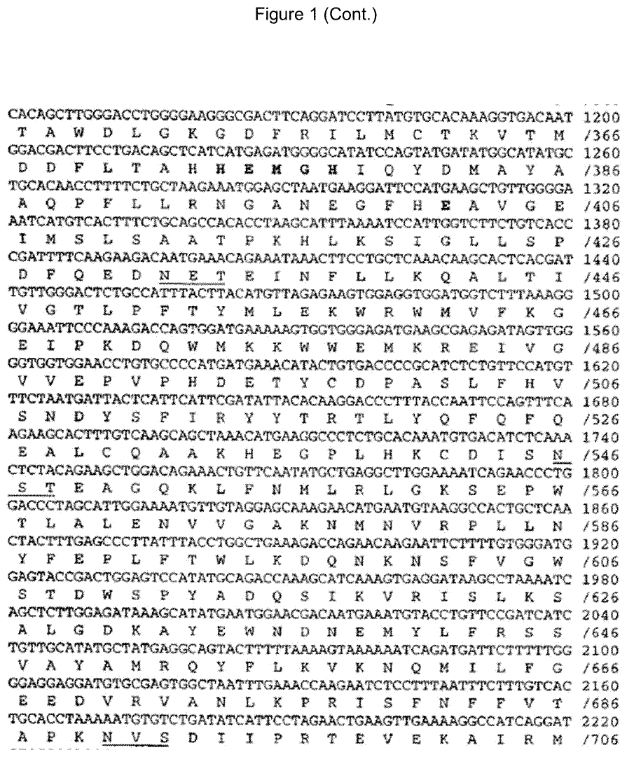

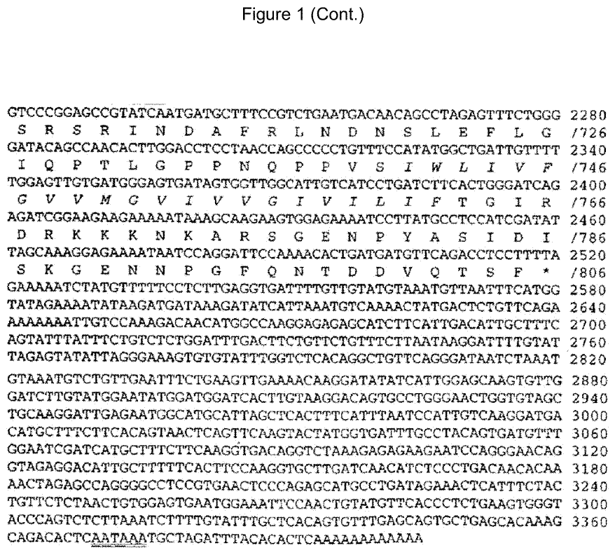

This figure sets forth the nucleotide and predicted amino acid sequence of human TMPRSS2 (GenBank Accession No. U75329). The potential initiation methionine codon and the translation stop codon are bold and underlined. The trapped sequences are underlined (for example the trapped sequence HMC26A01 extending from nucleotide 740 to 955). The different domains of the predicted polypeptide are dotted underlined (for example the SRCR domain extends from amino acid residue 148 to 242). The locations of the introns are shown with arrows. (Figure from, and text adapted from, FIG. 1 of A. Paoloni-Giacobino, et al.)

FIG. 3

This figure sets forth the characterization of SARS-CoV-2 RBD. It shows multiple sequence alignment of RBDs of SARS-CoV-2, SARS-CoV, and MERS-CoV spike (S) proteins. GenBank accession numbers are QHR63250.1 (SARS-CoV-2 S), AY278488.2 (SARS-CoV S), and AFS88936.1 (MERS-CoV S). Variable amino acid residues between SARS-CoV-2 and SARS-CoV are highlighted in dark grey (cyan), and conserved residues among SARS-CoV-2, SARS-CoV, and MERS-CoV are highlighted in light grey (yellow). Asterisks represent fully conserved residues, colons represent highly conserved residues, and periods represent lowly conserved residues. (Figure from, and text adapted from, FIG. 1(a) of Tai, et al.).

FIGS. 4A-4D

Each of FIGS. 4A, 4B, and 4C shows a schematic diagram of two expression cassettes for inclusion in two AAV-antibody vectors. In FIG. 4A, both vectors are needed for the expression of a single bispecific antibody (e.g., an IgG(kih) that comprises heavy chain 1 (HC1) and light chain 1 (LC1) (that together bind to a first epitope such as hACE2) and heavy chain 2 (HC2) and light chain 2 (LC2) (that together bind to a second epitope such as TMPRSS2). In FIG. 4B, both vectors are needed for the expression of a single bispecific antibody (e.g., an IgG(kih) that comprises heavy chain 1 (HC1) and a light chain (LC) (that together bind to a first epitope such as hACE2) and heavy chain 2 (HC2) and the same light chain (LC) (that together bind to a second epitope such as TMPRSS2). In FIG. 4C, both vectors are needed for the expression of a single bispecific antibody (e.g., an IgG(kih) that comprises heavy chain 1 (HC1) and a common light chain (LC) (that together bind to a first epitope such as hACE2) and heavy chain 2 (HC2) (that, together with the common light chain (LC), bind to a second epitope such as TMPRSS2). FIG. 4D shows a schematic diagram of an expression cassette for inclusion in an AAV-antibody vector. Only one vector is needed for the expression of a bispecific antibody (e.g., a tandem scFv (taFv) bispecific antibody that comprises the four antigen-binding segments Fv1 and Fv2 (that together bind to a first epitope such as hACE2) and Fv3 and Fv4 (that together bind to a second epitope such as hTMPRSS2)).

FIG. 5

This figure, taken from Du, et al., shows a humanization strategy for monoclonal antibody 11B11. Sequence alignments highlight the humanization strategy of murine 11B11, which strategy involves retaining all the CDRs and substituting the remaining amino acids with the corresponding residues of the human immunoglobulins. Human IGHV2-23*04, which exhibits high sequence identity to murine 11B11 in the heavy chain, was selected as the humanization backbone for the H chain, while IGKV2-39*01 was selected as the humanization backbone for the L chain. Panel (a) shows the heavy chain sequences, and panel (b) shows the light chain sequences. This description is adapted from Du, et al.

### DETAILED DESCRIPTION OF THE INVENTION

This invention provides certain bispecific antibodies that target both human ACE2 and TMPRSS2, as well as related engineered viruses. These antibodies and viruses are useful for therapeutically and prophylactically addressing SARS-CoV-2 infection.

### Definitions

In this application, certain terms are used which shall have the meanings set forth as follows.

As used herein, “administer”, with respect to antibodies, means to deliver the antibodies to a subject's body via any known method suitable for that purpose. Specific modes of administration include, without limitation, intravenous administration, intramuscular administration, and subcutaneous administration. Similarly, as used herein, “administer”, with respect to recombinant viral particles, means to deliver the particles to a subject's body via any known method suitable for that purpose. Specific modes of administration include, without limitation, intravenous administration, intramuscular administration, and subcutaneous administration.

In this invention, antibodies can be formulated using one or more routinely used pharmaceutically acceptable carriers. Such carriers are well known to those skilled in the art. For example, injectable drug delivery systems include solutions containing salts (e.g., sodium chloride and sodium phosphate). In a specific embodiment, the injectable drug delivery system comprises antibody (e.g., 100 mg, 200 mg, 300 mg, 400 mg, or 500 mg) in the form of a lyophilized powder in a multi-use vial, which is then reconstituted and diluted in, for example, 0.9% Sodium Chloride Injection, USP. In another specific embodiment, the injectable drug delivery system comprises antibody (e.g., 100 mg/50 ml, 200 mg/50 ml, 300 mg/50 ml, 400 mg/50 ml, or 500 mg/50 ml) in the form of a suspension in a single-use vial, which is then withdrawn and diluted in, for example, 0.9% Sodium Chloride Injection, USP. Injectable drug delivery systems also include suspensions, gels, microspheres and polymeric injectables, and can comprise excipients such as solubility-altering agents (e.g., ethanol, propylene glycol, and sucrose) and polymers (e.g., polycaprylactones and PLGAs).

In addition, in this invention, recombinant viral particles can be formulated using one or more routinely used pharmaceutically acceptable carriers. Such carriers are well known to those skilled in the art. For example, injectable drug delivery systems include solutions containing salts (e.g., sodium chloride and sodium phosphate) and surfactants (e.g., a poloxamer). In a specific embodiment, the injectable drug delivery system comprises an aqueous solution of sodium chloride (e.g., 180 mM), sodium phosphate (e.g., 10 mM), and a poloxamer (e.g., 0.001% Poloxamer 188). Injectable drug delivery systems also include suspensions, gels, microspheres and polymeric injectables, and can comprise excipients such as solubility-altering agents (e.g., ethanol, propylene glycol, and sucrose) and polymers (e.g., polycaprylactones and PLGAs).

As used herein, the term “antibody” includes, without limitation, (a) an immunoglobulin molecule comprising two heavy chains (i.e., H chains, such as μ, δ, γ, α and ε) and two light chains (i.e., L chains, such as λ and κ) and which recognizes an antigen; (b) polyclonal and monoclonal immunoglobulin molecules; (c) monovalent (e.g., Fab) and divalent fragments thereof, and (d) bispecific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4 (preferably, in this invention, IgG2, IgG4, or a combination of IgG2 and IgG4). Antibodies can be both naturally occurring and non-naturally occurring.

Furthermore, antibodies include chimeric antibodies, wholly synthetic antibodies, single chain antibodies (e.g., scFv), and fragments thereof. Antibodies may contain, for example, all or a portion of a constant region (e.g., an Fc region) and a variable region, or contain only a variable region (responsible for antigen binding). Antibodies may be human, humanized, chimeric, or nonhuman. Methods for designing and making human and humanized antibodies are well known (See, e.g., Chiu and Gilliland; Lafleur, et al.). Antibodies include, without limitation, the present bispecific antibodies as defined herein.

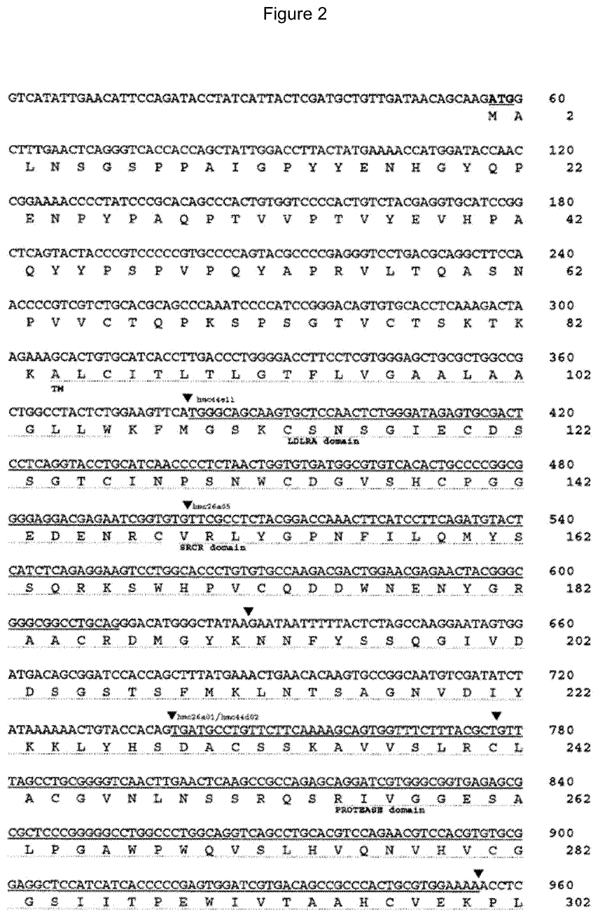

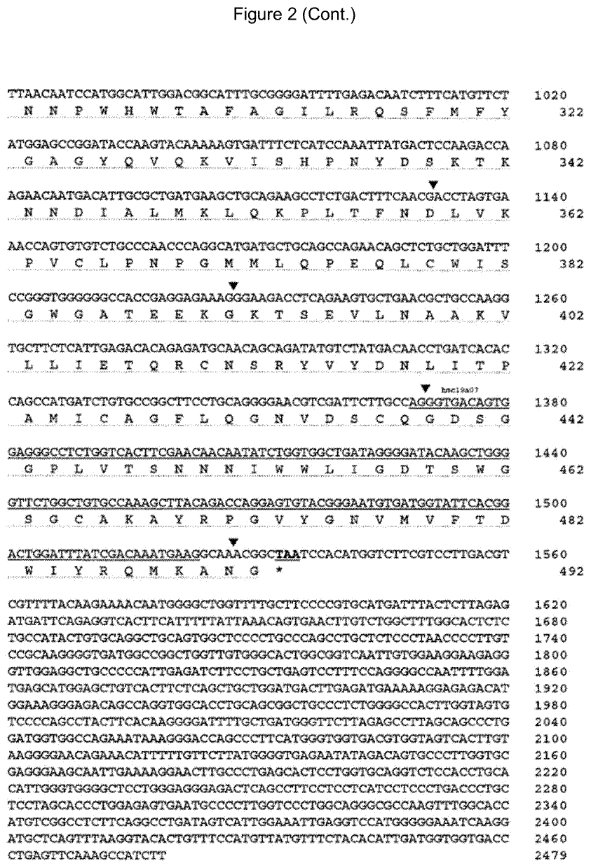

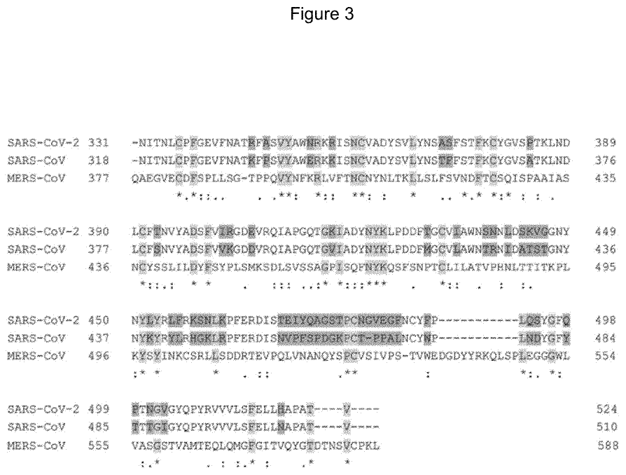

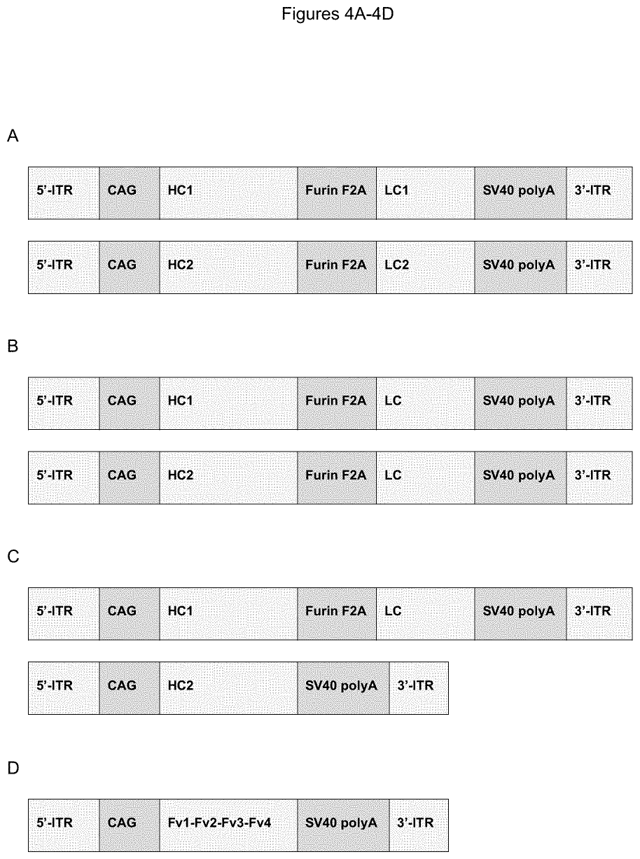

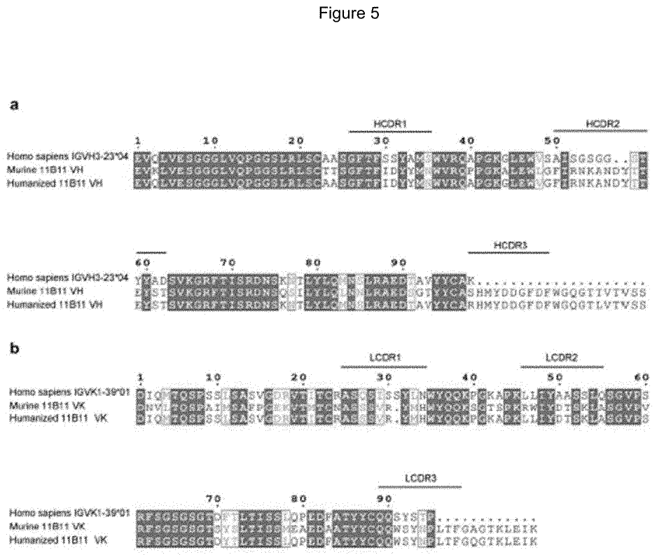

As used herein, a “bispecific antibody” includes, without limitation, an antibody that specifically binds to two different epitopes either on the same or different antigens. Bispecific antibody types are numerous and include, without limitation, the following: (i) bispecific antibody conjugates (e.g., IgG2, F(ab′)₂, and CovX-Body); (ii) hybrid bispecific IgGs (e.g., IgG, mouse/rat chimeric IgG, and κ/λ-body common HC); (iii) “variable domain only” bispecific antibody molecules (e.g., tandem scFv (taFv), triplebody, Diabody (db), dsDb, db(kih), DART, scDb, dsFv-dsFv′, tandAbs, triple heads, tandem dAb/VHH, triple dAb/VHH, and tetravalent dAb/VHH); (iv) CH1/CL fusion proteins (e.g., scFv2-CH1/CL and VHH2-CH1/CL); (v) Fab fusion proteins (e.g., Fab-scFv (bibody), Fab-scFv₂ (tribody), Fab-Fv, Fab-dsFv, Fab-VHH, and orthogonal Fab-Fab); (vi) non-immunoglobulin fusion proteins (e.g., scFv₂-album in, scDb-album in, taFv-album in, taFv-toxin, miniantibody, DNL-Fab₃, DNL-Fab₂-scFv, DNL-Fab₂-IgG-cytokine₂, and ImmTAC (TCR-scFv)); (vii) Fc-modified IgGs (e.g., IgG(kih), IgG(kih) common LC, ZW1 IgG common LC, Biclonics common LC, CrossMab (IgG-kih), scFab-IgG(kih), Fab-scFab-IgG(kih), orthogonal Fab IgG(kih), DuetMab, CH3 charge pairs+CH1/CL charge pairs, hinge/CH3 charge pairs, Duobody, four-in-one CrossMab (kih), LUZ-Y common LC, LUZ-Y scFab-IgG, and FcFc*); (viii) appended and Fc-modified IgGs (e.g., IgG(kih)-Fv, IgG(HA-TA)-Fv, IgG(kih)-scFab, scFab-Fc(kih)-scFv2, scFab-Fc(kih)-scFv, half DVD-Ig, DVI-Ig (four-in-one), and CrossMab-Fab); (ix) modified Fc and CH3 fusion proteins (e.g., scFv-Fc(kih), scFv-Fc (CH3 charge pairs), scFv-Fc (EW-RVT), scFv-Fc (HA-TF), scFv-Fc (SEEDbody), taFv-Fc (kih), scFv-Fc(kih)-Fv, Fab-Fc(kih)-scFv, Fab-scFv-Fc(kih), Fab-scFv-Fc(BEAT), Fab-scFv-Fc(SEEDbody), DART-Fc, scFv-CH3(kih), and TriFabs); (x) appended IgGs-HC fusions (e.g., IgG-HC-scFv, IgG-dAb, IgG-taFv, IgG-CrossFab, IgG-orthogonal Fab, IgG-(CαCβ) Fab, scFv-HC-IgG, tandem Fab-IgG (orthogonal Fab), Fab-IgG(CαCβFab), Fab-IgG(CR3), and Fab-hinge-IgG(CR3); (xi) appended IgGs-LC fusions (e.g., IgG-scFv(LC), scFv(LC)-IgG, and dAb-IgG); (xii) appended IgGs-HC and LC fusions (e.g., DVD-Ig, TVD-Ig, CODV-Ig, scFv₄-Ig, and Zybody); (xiii) Fc fusions (e.g., Di-diabody, scDb-Fc, taFv-Fc, scFv-Fc-scFv, HCAb-VHH, Fab-scFv-Fc, scFv₄-Ig, and scFv₂-Fcab); (xiv) CH3 fusions (e.g., Di-diabody and scDb-C_{H}3); (xv) IgE/IgM CH2 fusions (e.g., scFv-EHD2-scFv and scFv-MHD2-scFv); (xvi) F(ab′)₂ fusions (e.g., F(ab′)₂-scFv₂); (xvii) CH1/CL fusion proteins (e.g., scFv₂-CH1-hinge/CL); (xviii) modified IgGs (e.g., DAF (two-in-one-IgG), DutaMab, and mAb²); and (xix) non-immunoglobulin fusions (e.g., DNL-Fab₄-IgG). A chart illustrating these bispecific antibody types is found in FIG. 2 of Brinkmann, et al.

As used herein, “CDR1” shall mean complementarity-determining region 1, which includes heavy chain CDR1 and light chain CDR1. “CDR2” shall mean complementarity-determining region 2, which includes heavy chain CDR2 and light chain CDR2. Finally, “CDR3” shall mean complementarity-determining region 3, which includes heavy chain CDR3 and light chain CDR3.

As used herein, “effector function”, with respect to an antibody, includes, without limitation, antibody-dependent cell-mediated cytotoxicity (ADCC), antibody-dependent cellular phagocytosis (ADCP), and complement fixation.

As used herein, the present bispecific antibody binds to an hACE2 “epitope” comprising a given amino acid residue if, for example, that residue directly contacts (e.g., via a hydrogen bond) at least one amino acid residue in the antibody's paratope.

As used herein, the present bispecific antibody binds to an hTMPRSS2 “epitope” comprising a given amino acid residue if, for example, that residue directly contacts (e.g., via a hydrogen bond) at least one amino acid residue in the antibody's paratope.

As used herein, a subject who has been “exposed” to SARS-CoV-2 includes, for example, a subject who experienced a high-risk event (e.g., one in which he/she came into contact with the bodily fluids of an infected human subject, such as by inhaling droplets of virus-containing saliva or touching a virus-containing surface). In one embodiment, this exposure occurs two weeks, one week, five days, four days, three days, two days, one day, six hours, two hours, one hour, or 30 minutes prior to receiving the subject prophylaxis.

As used herein, “human angiotensin converting enzyme 2”, also referred to herein as “hACE2”, shall mean (i) the protein having the amino acid sequence set forth in FIG. 1; or (ii) a naturally occurring human variant thereof (e.g., the I21T variant, the N33D variant, the D38E variant, and the K26R variant). In a preferred embodiment, hACE2 shall mean the protein having the amino acid sequence set forth in FIG. 1.

As used herein, a “human subject” can be of any age, gender, or state of co-morbidity. In one embodiment, the subject is male, and in another, the subject is female. In another embodiment, the subject is co-morbid (e.g., afflicted with diabetes, asthma, and/or heart disease). In a further embodiment, the subject is not co-morbid. In still another embodiment, the subject is younger than 60 years old. In yet another embodiment, the subject is at least 60 years old, at least 65 years old, at least 70 years old, at least 75 years old, at least 80 years old, at least 85 years old, or at least 90 years old.

As used herein, “human TMPRSS2”, also referred to herein as “hTMPRSS2”, shall mean (i) the protein having the amino acid sequence set forth in FIG. 2; or (ii) a naturally occurring human variant thereof. Human TMPRSS2 is also known in the art as epitheliasin, and as transmembrane protease, serine 2. hTMPRSS2 cleaves the SARS-CoV-2 S protein. Without wishing to be bound by any particular theory of hTMPRSS2 function, it is believed that hTMPRSS2 cleaves SARS-CoV-2 S protein at an “S1/S2” cleavage site (i.e., between amino acid residues R685 and S686) and an “S2” cleavage site (i.e., between amino acid residues R815 and S816). See, e.g., Coutard, et al.

As used herein, a subject is “infected” with a virus if the virus is present in the subject. Present in the subject includes, without limitation, present in at least some cells in the subject, and/or present in at least some extracellular fluid in the subject. In one embodiment, the virus present in the subject's cells is replicating. A subject who is exposed to a virus may or may not become infected with it.

Heavy chain modifications that “inhibit half antibody formation” in IgG4 are described, for example, in C. Dumet, et al. They include, without limitation, the following, with numbering according to the EU Index: (i) S228P; (ii) the mutation combination S228P/R409K; and (iii) K447del and the mutation combination S228P/K447del. Related heavy chain modifications that solve the heavy chain-mispairing problem include, for example, the “knobs-into-holes” (kih) modifications described in M. Godar, et al., and WO/1996/027011.

As used herein, a “long serum half-life”, with respect to a bispecific antibody, is a serum half-life of at least five days (preferably as measured in vivo in a human, but which may also be measured, for example, in mice, rats, rabbits, and monkeys (e.g., rhesus monkeys, cynamolgous macaques, and marmosets)). In a preferred embodiment, a bispecific antibody has a long serum half-life if its half-life is at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 90 days, at least 95 days, or at least 100 days. In another preferred embodiment, a bispecific antibody has a long serum half-life if its half-life is from 15 days to 20 days, from 20 days to 25 days, from 25 days to 30 days, from 30 days to 35 days, from 35 days to 40 days, from 40 days to 45 days, from 45 days to 50 days, from 50 days to 55 days, from 55 days to 60 days, from 60 days to 65 days, from 65 days to 70 days, from 70 days to 75 days, from 75 days to 80 days, from 80 days to 85 days, from 85 days to 90 days, from 90 days to 95 days, from 95 days to 100 days, or over 100 days. Examples of bispecific IgG heavy chain modifications that increase half-life relative to corresponding wild-type IgG heavy chains (such as those that increase antibody binding to FcRn) are described in C. Dumet, et al. and G. J. Robbie, et al. They include, without limitation, the following, with numbering according to the EU Index: (i) point mutations at position 252, 254, 256, 309, 311, 433, 434, and/or 436, including the “YTE” mutation combination M252Y/S254T/T256E (U.S. Pat. No. 7,083,784); (ii) the “LS” mutation combination M428L/N434S (WO/2009/086320); (iii) the “QL” mutation combination T250Q/M428L; and (iv) the mutation combinations M428L/V308F and Q311V/N434S.

As used herein, a bispecific antibody having a “low effector function” includes, without limitation, (i) a bispecific antibody that has no effector function (e.g., by virtue of having no Fc domain), and (ii) a bispecific antibody that has a moiety (e.g., a modified Fc domain) possessing an effector function lower than that of a wild-type IgG1 antibody. Bispecific antibodies having a low effector function include, for example, a tandem scFv bispecific antibody, and a bispecific IgG4 antibody (e.g., a bispecific IgG4 antibody having heavy chains engineered to reduce effector function relative to wild-type IgG4 heavy chains). An example of a bispecific IgG1 heavy chain modification that lowers effector function relative to wild-type IgG1 heavy chains is the L234A/L235A/P329G (LALA-PG) modification described in Ferarri, et al., with numbering according to the EU Index. Examples of bispecific IgG4 heavy chain modifications that lower effector function relative to wild-type IgG4 heavy chains are described in C. Dumet, et al. They include, without limitation, the following, with numbering according to the EU Index: (i) L235E (WO/1994/028027); (ii) L235A, F234A, and G237A (WO/1994/029351 and WO/1995/026403); (iii) D265A (U.S. Pat. No. 7,332,581); (iv) L328 substitution, A330R, and F243L (WO/2004/029207); (v) IgG2/IgG4 format wherein IgG2 (up to T260) is joined to IgG4 (WO/2005/007809); (vi) F243A/V264A combination (WO/2011/149999); (vii) E233P/F234A/L235A/G236del/G237A combination (WO/2017/079369); and (viii) S228P/L235E combination. Examples of such IgG4 heavy chain modifications are also described in T. Schlothauer, et al., and include, without limitation, S228P/L235E/P329G (SPLE P329G), with numbering according to the EU Index.

As used herein, the “normal function” of hACE2 includes, without limitation, at least one of the following: (i) the ability to convert angiotensin II to angiotensin-(1-7) (i.e., by enzymatically cleaving the C-terminal phenylalanine residue from angiotensin II to form angiotensin-(1-7)); (ii) the ability to cleave [des-Arg]-bradykinin (also known as [des-Arg⁹]-bradykinin); (iii) the ability to hydrolyze Aβ-43 to yield Aβ-42; (iv) the ability to convert angiotensin I to angiotensin-(1-9); (v) the ability to cleave neurotensin; (vi) the ability to cleave kinetensin; (vii) the ability to cleave a synthetic MCA-based peptide; (viii) the ability to cleave apelin-13; and (ix) the ability to cleave dynorphin A 1-13. In one embodiment, the normal function of hACE2 means (i) the ability to convert angiotensin II to angiotensin-(1-7); (ii) the ability to cleave [des-Arg]-bradykinin; (iii) the ability to hydrolyze Aβ-43 to yield Aβ-42; (iv) the ability to convert angiotensin I to angiotensin-(1-9); (v) the ability to cleave neurotensin; (vi) the ability to cleave kinetensin; (vii) the ability to cleave a synthetic MCA-based peptide; (viii) the ability to cleave apelin-13; and (ix) the ability to cleave dynorphin A 1-13. In a preferred embodiment, the normal function of hACE2 means the ability to convert angiotensin II to angiotensin-(1-7). By way of example, hACE2 activity can be measured using angiotensin II as a substrate to yield angiotensin-(1-7) according to known methods using known reagents, as described in the examples below. hACE2 activity can also be measured using a synthetic MCA-based peptide (e.g., a Mc-Ala/Dnp fluorescence resonance energy transfer (FRET) peptide that yields Mc-Ala upon cleavage by hACE2) according to known methods using known reagents, as described in the examples below.

As used herein, a “prophylactically effective amount” of the present antibodies includes, without limitation, (i) 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 150 mg, 200 mg, 250 mg, 300 mg, 350 mg, 400 mg, 450 mg, 500 mg, 600 mg, 700 mg, 800 mg, 900 mg, 1 g, 2 g, 3 g, 4 g, 5 g, 6 g, 7 g, 8 g, 9 g, or 10 g; (ii) 5 mg to 20 mg, 20 mg to 50 mg, 50 mg to 100 mg, 100 mg to 200 mg, 200 mg to 300 mg, 300 mg to 400 mg, 400 mg to 500 mg, 500 mg to 1 g, 1 g to 2 g, 2 g to 5 g, or 5 g to 10 g; (iii) 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg; or (iv) 1 mg/kg to 10 mg/kg, 10 mg/kg to 20 mg/kg, 20 mg/kg to 30 mg/kg, 30 mg/kg to 40 mg/kg, 40 mg/kg to 50 mg/kg, 50 mg/kg to 100 mg/kg, 75 mg/kg to 125 mg/kg, 100 mg/kg to 150 mg/kg, or 150 mg/kg to 200 mg/kg. In the preferred embodiment, the prophylactically effective amount of antibodies is administered as a single, one-time-only dose. In another embodiment, the prophylactically effective amount of antibodies is administered as two or more doses over a period of days, weeks, or months (e.g., twice daily for one or two weeks; once daily for one or two weeks; every other day for two weeks; three times per week for two weeks; twice per week for two weeks; once per week for two weeks; twice with the administrations separated by two weeks; once per month; once every two months; once every three months; once every four months; twice per year; or once per year).

As used herein, a “prophylactically effective amount” of the present recombinant viral particles (e.g., recombinant AAV particles) includes, without limitation, (i) from 1×10¹⁰ to 5×10¹⁰ particles (also referred to as “viral genomes” or “vg”) per kg of body weight, from 5×10¹⁰ to 1×10¹¹ particles/kg, from 1×10¹¹ to 5×10¹¹ particles/kg, from 5×10¹¹ to 1×10¹² particles/kg, from 1×10¹² to 5×10¹² particles/kg, from 5×10¹² to 1×10¹³ particles/kg, from 1×10¹³ to 5×10¹³ particles/kg, or from 5×10¹³ to 1×10¹⁴ particles/kg; or (ii) 1×10¹⁰ particles/kg, 5×10¹⁰ particles/kg, 1×10″ particles/kg, 5×10″ particles/kg, 1×10¹² particles/kg, 5×10¹² particles/kg, 1×10¹³ particles/kg, 5×10¹³ particles/kg, or 1×10¹⁴ particles/kg, 5×10¹⁴ particles/kg, or 1×10¹⁵ particles/kg. In the preferred embodiment, the prophylactically effective amount of viral particles is administered as a single, one-time-only dose. In another embodiment, the prophylactically effective amount of viral particles is administered as two or more doses over a period of months or years.

As used herein, a “recombinant AAV (adeno-associated virus) particle”, also referred to as “rAAV particle”, includes, without limitation, an AAV capsid protein (e.g., VP1, VP2 and/or VP3) and a vector comprising a nucleic acid encoding an exogenous protein (e.g., an antibody heavy chain) situated between a pair of AAV inverted terminal repeats in a manner permitting the AAV particle to infect a target cell. Preferably, the recombinant AAV particle is incapable of replication within its target cell. The AAV serotype may be any AAV serotype suitable for use in gene therapy, such as AAV1, AAV2, AAV3, AAV4, AAV5, AAV6, AAV7, AAV8, AAV9, AAV10, AAVrh10, AAV11, AAV12, LK01, LK02 or LK03.

As used herein, “reducing the likelihood” of a human subject's becoming infected with a virus includes, without limitation, reducing such likelihood by at least 10%, at least 20%, at least 30%, at least 40%, at least 50%, at least 60%, at least 70%, at least 80%, at least 90%, at least 95%, or at least 99%. Preferably, reducing the likelihood of a human subject's becoming infected with a virus means preventing the subject from becoming infected with it. Similarly, “reducing the likelihood” of a human subject's becoming symptomatic of a viral infection includes, without limitation, reducing such likelihood by at least 10%, at least 20%, at least 30%, at least 40%, at least 50%, at least 60%, at least 70%, at least 80%, at least 90%, at least 95%, or at least 99%. Preferably, reducing the likelihood of a human subject's becoming symptomatic of a viral infection means preventing the subject from becoming symptomatic.

As used herein, “SARS-CoV-2” includes, without limitation, the following variants: Wuhan-1; F338L; A348T; N354D; N354K; V367F; R408I; Q409E; Q414E; G446V; L452R; K458N; K458R; I468T; A475V; T478I; V483A; V483I; E484K; N501Y; Y508H; H519P; H519Q; A520S; V615L; P1263L; D614G+69-70del; D614G+A262S; D614G+V341 I; D614G+Q321L; D614G+K417N; D614G+N439K; D614G+Y453F; D614G+S477N; and D614G+F486L.

As used herein, an antibody does not “significantly inhibit the ability of hACE2 to cleave” a substrate if (i) it inhibits the ability of intact hACE2 (i.e., full-length hACE2 that includes the extracellular portion, transmembrane portion, and intracellular portion) to cleave the substrate by less than 90%, and/or (ii) it inhibits the ability of the extracellular portion of hACE2 (e.g., recombinant soluble hACE2) to cleave the substrate by less than 90%. In one embodiment, an antibody does not significantly inhibit the ability of hACE2 to cleave a substrate if it inhibits the ability of intact hACE2 to cleave the substrate by less than 90%. In another embodiment, an antibody does not significantly inhibit the ability of hACE2 to cleave a substrate if it inhibits the ability of the extracellular portion of hACE2 to cleave the substrate by less than 90%. Preferably, an antibody does not significantly inhibit the ability of hACE2 (i.e., intact hACE2 and/or its extracellular portion) to cleave a substrate if it inhibits that ability by less than 80%, less than 70%, less than 60%, less than 50%, less than 40%, less than 30%, less than 20%, less than 10%, less than 5%, or less than 1%. By way of example, an antibody does not significantly inhibit the ability of hACE2 (i.e., intact hACE2 and/or its extracellular portion) to cleave angiotensin II if it inhibits that ability by less than 90%, less than 80%, less than 70%, less than 60%, less than 50%, less than 40%, less than 30%, less than 20%, less than 10%, less than 5%, or less than 1%. By way of further example, an antibody does not significantly inhibit the ability of hACE2 (i.e., intact hACE2 and/or its extracellular portion) to cleave des-Arg-bradykinin if it inhibits that ability by less than 90%, less than 80%, less than 70%, less than 60%, less than 50%, less than 40%, less than 30%, less than 20%, less than 10%, less than 5%, or less than 1%. By way of further example, an antibody does not significantly inhibit the ability of hACE2 (i.e., intact hACE2 and/or its extracellular portion) to cleave neurotensin if it inhibits that ability by less than 90%, less than 80%, less than 70%, less than 60%, less than 50%, less than 40%, less than 30%, less than 20%, less than 10%, less than 5%, or less than 1%. By way of further example, an antibody does not significantly inhibit the ability of hACE2 (i.e., intact hACE2 and/or its extracellular portion) to cleave kinetensin if it inhibits that ability by less than 90%, less than 80%, less than 70%, less than 60%, less than 50%, less than 40%, less than 30%, less than 20%, less than 10%, less than 5%, or less than 1%. By way of further example, an antibody does not significantly inhibit the ability of hACE2 (i.e., intact hACE2 and/or its extracellular portion) to cleave a synthetic MCA-based peptide (preferably Mca-APK(Dnp)) if it inhibits that ability by less than 90%, less than 80%, less than 70%, less than 60%, less than 50%, less than 40%, less than 30%, less than 20%, less than 10%, less than 5%, or less than 1%. By way of further example, an antibody does not significantly inhibit the ability of hACE2 (i.e., intact hACE2 and/or its extracellular portion) to cleave apelin-13 if it inhibits that ability by less than 90%, less than 80%, less than 70%, less than 60%, less than 50%, less than 40%, less than 30%, less than 20%, less than 10%, less than 5%, or less than 1%. By way of further example, an antibody does not significantly inhibit the ability of hACE2 (i.e., intact hACE2 and/or its extracellular portion) to cleave dynorphin A 1-13 if it inhibits that ability by less than 90%, less than 80%, less than 70%, less than 60%, less than 50%, less than 40%, less than 30%, less than 20%, less than 10%, less than 5%, or less than 1%.

As used herein, an antibody does not “significantly inhibit” the ability of a protease to cleave a substrate if it inhibits the ability of the protease to cleave the substrate by less than 90%. The protease in this context can be, for example, (i) an intact transmembrane protease that comprises an extracellular portion, a transmembrane portion, and an intracellular portion, (ii) a recombinant solubilized extracellular portion of an intact transmembrane protease, or (iii) a naturally soluble protease. Preferably, an antibody does not significantly inhibit the ability of a protease to cleave a substrate if it inhibits that ability by less than 80%, less than 70%, less than 60%, less than 50%, less than 40%, less than 30%, less than 20%, less than 10%, less than 5%, or less than 1%. In another preferred embodiment, an antibody does not significantly inhibit the ability of one or more of human TMPRSS1 (also known as hepsin; transmembrane protease, serine 1; TADG-12; and HPN), human TMPRSS3 (also known as transmembrane protease, serine 3; and TADG-12), human TMPRSS4 (also known as transmembrane protease, serine 4; transmembrane protease, serine 3; TMPRSS3; and MT-SP2), human TMPRSS5 (also known as transmembrane protease, serine 5; and spinesin), human TMPRSS6 (also known as transmembrane protease, serine 6; and matripase-2), human TMPRSS7 (also known as transmembrane protease, serine 7; and matripase-3), human TMPRSS9 (also known as transmembrane protease, serine 9; and polyserase-1), human TMPRSS10 (also known as transmembrane protease, serine 10; corin; and Lrp4), human TMPRSS11A (also known as transmembrane protease, serine 11A; DESC3; differentially expressed in squamous cell carcinoma-3; HAT-like 1; and HATL1), human TMPRSS11B (also known as transmembrane protease, serine 11B; and HAT-like 5), human TMPRSS11C (also known as transmembrane protease, serine 11C; HAT-like 3; and neurobin), human TMPRSS11D (also known as transmembrane protease, serine 11D; HAT; human airway trypsin-like protease; adrenal serine protease; and asp), human TMPRSS11E (also known as transmembrane protease, serine 11E; DESC1; and differentially expressed in squamous cell carcinoma-1), human TMPRSS11F (also known as transmembrane protease, serine 11F; and HAT-like 4), human enteropeptidase (also known as PRSS7; protease; serine 7; and enterokinase) and human matriptase (also known as MT-SP1; epithin; PRSS14; protease; serine 14; TADG-15; ST14; and SNC19) to cleave a substrate if it inhibits that ability by less than 80%, less than 70%, less than 60%, less than 50%, less than 40%, less than 30%, less than 20%, less than 10%, less than 5%, or less than 1%.

In still another preferred embodiment, an antibody does not significantly inhibit the ability of any of human TMPRSS1, human TMPRSS3, human TMPRSS4, human TMPRSS5, human TMPRSS6, human TMPRSS7, human TMPRSS9, human TMPRSS10, human TMPRSS11A, human TMPRSS11B, human TMPRSS11C, human TMPRSS11D, human TMPRSS11E, human TMPRSS11F, human enteropeptidase and human matriptase to cleave a substrate if it inhibits that ability by less than 80%, less than 70%, less than 60%, less than 50%, less than 40%, less than 30%, less than 20%, less than 10%, less than 5%, or less than 1%. By way of example, an antibody does not significantly inhibit the ability of human TMPRSS1 (i.e., intact human TMPRSS1 and/or its extracellular portion) to cleave its substrate if it inhibits that ability by less than 90%, less than 80%, less than 70%, less than 60%, less than 50%, less than 40%, less than 30%, less than 20%, less than 10%, less than 5%, or less than 1%.

As used herein, an antibody “specifically binds” to the extracellular portion of hACE2 if it does at least one of the following: (i) binds to the extracellular portion of hACE2 with an affinity greater than that with which it binds to any other human cell surface protein; or (ii) binds to the extracellular portion of hACE2 with an affinity of at least 500 μM. Preferably, an antibody specifically binds to the extracellular portion of hACE2 if it performs both of items (i) and (ii) above. In a preferred embodiment, the antibody binds to hACE2 (i.e., to its extracellular portion) with an affinity of at least 100 μM, at least 10 μM, at least 1 μM, at least 500 nM, at least 300 nM, at least 200 nM, at least 100 nM, at least 50 nM, at least 20 nM, at least 10 nM, at least 5 nM, at least 1 nM, at least 0.5 nM, at least 0.1 nM, at least 0.05 nM, or at least 0.01 nM. In a preferred embodiment, the present antibody binds to hACE2 with an affinity greater than that with which SARS-CoV-2 RBD binds to hACE2.

As used herein, an antibody “specifically binds” to the extracellular portion of hTMPRSS2 if it does at least one of the following: (i) binds to the extracellular portion of hTMPRSS2 with an affinity greater than that with which it binds to any other human cell surface protein (including, without limitation, any other transmembrane protease); or (ii) binds to the extracellular portion of hTMPRSS2 with an affinity of at least 500 μM. Preferably, an antibody specifically binds to the extracellular portion of hTMPRSS2 if it performs both of items (i) and (ii) above. In a preferred embodiment, the antibody binds to the extracellular portion of hTMPRSS2 with an affinity of at least 100 μM, at least 10 μM, at least 1 μM, at least 500 nM, at least 300 nM, at least 200 nM, at least 100 nM, at least 50 nM, at least 20 nM, at least 10 nM, at least 5 nM, at least 1 nM, at least 0.5 nM, at least 0.1 nM, at least 0.05 nM, or at least 0.01 nM. In another preferred embodiment, the antibody binds to the extracellular portion of hTMPRSS2 with an affinity of at least 100 μM, but does not bind to any other human cell surface protein with an affinity greater than 200 μM. In another preferred embodiment, the antibody, by binding to the extracellular portion of hTMPRSS2, “knocks out” hTMPRSS2 (i.e., eliminates all enzymatic function of hTMPRSS2).

As used herein, an antibody “specifically inhibits” binding of SARS-CoV-2 to the extracellular portion of hACE2 if it does at least one of the following: (i) reduces such binding more than it reduces the binding of SARS-CoV-2 to any other human cell surface protein; or (ii) reduces such binding by a factor of at least two. Preferably, an antibody specifically inhibits binding of SARS-CoV-2 to the extracellular portion of hACE2 if it performs both of items (i) and (ii) above. In a preferred embodiment, the antibody reduces binding of SARS-CoV-2 to the extracellular portion of hACE2 by a factor of at least 10, at least 20, at least 50, at least 100, at least 1,000, at least 10,000, at least 100,000, or at least 1,000,000.

As used herein, an antibody “specifically inhibits” binding of the SARS-CoV-2 S1 protein receptor binding domain fragment, also referred to as the RBD (e.g., the protein consisting of S amino acid residues 331 to 524) to the extracellular portion of hACE2 if it does at least one of the following: (i) reduces such binding more than it reduces the binding of SARS-CoV-2 S1 protein receptor binding domain fragment to any other human cell surface protein; or (ii) reduces such binding by a factor of at least two. Preferably, an antibody specifically inhibits binding of SARS-CoV-2 S1 protein receptor binding domain fragment to the extracellular portion of hACE2 if it performs both of items (i) and (ii) above. In a preferred embodiment, the antibody reduces binding of SARS-CoV-2 S1 protein receptor binding domain fragment to the extracellular portion of hACE2 by a factor of at least 10, at least 20, at least 50, at least 100, at least 1,000, at least 10,000, at least 100,000, or at least 1,000,000.

As used herein, an antibody “specifically inhibits” cleavage of SARS-CoV-2 S protein by hTMPRSS2 if it does at least one of the following: (i) reduces such cleavage more than it reduces the cleavage of SARS-CoV-2 S protein by any other human cell surface protease (e.g., any other human TMPRSS protease); or (ii) reduces such cleavage by a factor of at least two. Preferably, an antibody specifically inhibits cleavage of SARS-CoV-2 S protein by hTMPRSS2 if it performs both of items (i) and (ii) above. In a preferred embodiment, the antibody reduces cleavage of SARS-CoV-2 S protein by hTMPRSS2 by a factor of at least 10, at least 20, at least 50, at least 100, at least 1,000, at least 10,000, at least 100,000, or at least 1,000,000. In another preferred embodiment, the antibody does not significantly inhibit the ability of a protease, other than hTMPRSS2, to cleave a substrate.

As used herein, an antibody “specifically inhibits” the entry of SARS-CoV-2 into hACE2⁺/hTMPRSS2⁺ human cells if it does at least one of the following: (i) reduces such entry more than it reduces the entry of SARS-CoV-2 into hACE2⁻/hTMPRSS2⁻ human cells; or (ii) reduces such entry by a factor of at least two. Preferably, an antibody specifically inhibits the entry of SARS-CoV-2 into hACE2⁺/hTMPRSS2⁺ human cells if it performs both of items (i) and (ii) above. In a preferred embodiment, the antibody reduces the entry of SARS-CoV-2 into hACE2⁺/hTMPRSS2⁺ human cells by a factor of at least 10, at least 20, at least 50, at least 100, at least 1,000, at least 10,000, at least 100,000, or at least 1,000,000.

As used herein, an antibody “specifically inhibits” the entry into hACE2⁺/hTMPRSS2⁺ human cells of a pseudovirus (e.g., a replication-defective SARS-CoV-2 pseudovirus) bearing SARS-CoV-2 S protein if it does at least one of the following: (i) reduces such entry more than it reduces the entry into hACE2⁻/hTMPRSS2⁻ human cells of a pseudovirus bearing SARS-CoV-2 S protein; or (ii) reduces such entry by a factor of at least two. Preferably, an antibody specifically inhibits the entry into hACE2⁺/hTMPRSS2⁺ human cells of a pseudovirus bearing SARS-CoV-2 S protein if it performs both of items (i) and (ii) above. In a preferred embodiment, the antibody reduces the entry into hACE2⁺/hTMPRSS2⁺ human cells of a pseudovirus bearing SARS-CoV-2 S protein by a factor of at least 10, at least 20, at least 50, at least 100, at least 1,000, at least 10,000, at least 100,000, or at least 1,000,000.

As used herein, the term “subject” includes, without limitation, a mammal such as a human, a non-human primate, a dog, a cat, a horse, a sheep, a goat, a cow, a rabbit, a pig, a hamster, a rat and a mouse. The present methods are envisioned for these non-human embodiments, mutatis mutandis, as they are for human subjects in this invention.

As used herein, a human subject is “symptomatic” of a SARS-CoV-2 infection if the subject shows one or more symptoms known to appear in a SARS-CoV-2-infected human subject after a suitable incubation period. Such symptoms include, without limitation, detectable SARS-CoV-2 in the subject, and those symptoms shown by patients afflicted with COVID-19. COVID-19-related symptoms include, without limitation, fever, cough, shortness of breath, persistent pain or pressure in the chest, new confusion or inability to arouse, and/or bluish lips or face.

As used herein, a “synthetic MCA-based peptide” is a peptide having affixed at one end an MCA (i.e., (7-methoxycoumarin-4-yl)acetyl) moiety and having affixed at the other end a fluorescence-quenching moiety (e.g., 2,4-dinitrophenyl, which is also referred to as DNP or Dnp). Upon its enzymatic cleavage (e.g., by hACE2), the MCA-containing portion of the cleaved peptide is freed from the portion containing the fluorescence-quenching moiety. This, in turn, results in the now unquenched MCA-containing portion emitting a greater detectable fluorescent signal. As such, synthetic MCA-based peptides cleavable by hACE2 can serve as substrates permitting facile fluorescence-based measurement of hACE2 activity and its inhibition. In one embodiment, the synthetic MCA-based peptide comprises the consensus sequence Pro-X_{(1-3 residues)}-Pro-Hydrophobic Residue (e.g., MCA-Pro-X_{(1-3 residues)}-Pro-Hydrophobic Residue-DNP), whereby hACE2 cleaves between the proline and the hydrophobic residue. In another embodiment, the synthetic MCA-based peptide is MCA-YVADAPK-DNP (also referred to as Mca-YVADAPK(Dnp)). In a preferred embodiment, the synthetic MCA-based peptide is MCA-APK-DNP (also referred to as Mca-APK(Dnp)). In another preferred embodiment, the synthetic MCA-based peptide is the Mc-Ala/Dnp fluorescence resonance energy transfer (FRET) peptide used in the SensoLyte 390 ACE2 Activity Assay Kit *Fluorimetric* (Anaspec) described below. In yet another preferred embodiment, the synthetic MCA-based peptide is the ACE2 Substrate used in the Angiotensin II Converting Enzyme (ACE2) Activity Assay Kit (Fluorometric) (BioVision) described below.

As used herein, a “therapeutically effective amount” of the present antibodies includes, without limitation, (i) 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 150 mg, 200 mg, 250 mg, 300 mg, 350 mg, 400 mg, 450 mg, 500 mg, 600 mg, 700 mg, 800 mg, 900 mg, 1 g, 2 g, 3 g, 4 g, 5 g, 6 g, 7 g, 8 g, 9 g, or 10 g; (ii) 5 mg to 20 mg, 20 mg to 50 mg, 50 mg to 100 mg, 100 mg to 200 mg, 200 mg to 300 mg, 300 mg to 400 mg, 400 mg to 500 mg, 500 mg to 1 g, 1 g to 2 g, 2 g to 5 g, or 5 g to 10 g; (iii) 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg; or (iv) 1 mg/kg to 10 mg/kg, 10 mg/kg to 20 mg/kg, 20 mg/kg to 30 mg/kg, 30 mg/kg to 40 mg/kg, 40 mg/kg to 50 mg/kg, 50 mg/kg to 100 mg/kg, 75 mg/kg to 125 mg/kg, 100 mg/kg to 150 mg/kg, or 150 mg/kg to 200 mg/kg. In the preferred embodiment, the therapeutically effective amount of antibodies is administered as a single, one-time-only dose. In another embodiment, the therapeutically effective amount of antibodies is administered as two or more doses over a period of days, weeks, or months (e.g., twice daily for one or two weeks; once daily for one or two weeks; every other day for two weeks; three times per week for two weeks; twice per week for two weeks; once per week for two weeks; twice with the administrations separated by two weeks; once per month; once every two months; once every three months; once every four months; twice per year; or once per year).

As used herein, a “therapeutically effective amount” of the subject recombinant viral particles (e.g., recombinant AAV particles) includes, without limitation, (i) from 1×10¹⁰ to 5×10¹⁰ particles (also referred to as “viral genomes” or “vg”) per kg of body weight, from 5×10¹⁰ to 1×10¹¹ particles/kg, from 1×10¹¹ to 5×10¹¹ particles/kg, from 5×10¹¹ to 1×10¹² particles/kg, from 1×10¹² to 5×10¹² particles/kg, from 5×10¹² to 1×10¹³ particles/kg, from 1×10¹³ to 5×10¹³ particles/kg, or from 5×10¹³ to 1×10¹⁴ particles/kg; or (ii) 1×10¹⁰ particles/kg, 5×10¹⁰ particles/kg, 1×10¹¹ particles/kg, 5×10¹¹ particles/kg, 1×10¹² particles/kg, 5×10¹² particles/kg, 1×10¹³ particles/kg, 5×10¹³ particles/kg, or 1×10¹⁴ particles/kg, 5×10¹⁴ particles /kg, or 1×10¹⁵ particles/kg. In the preferred embodiment, the therapeutically effective amount of viral particles is administered as a single, one-time-only dose. In another embodiment, the therapeutically effective amount of viral particles is administered as two or more doses over a period of months or years.

As used herein, “treating” a subject afflicted with a disorder (e.g., a subject infected with SARS-CoV-2 and symptomatic of that infection) includes, without limitation, (i) slowing, stopping, or reversing the progression of one or more of the disorder's symptoms, (ii) slowing, stopping or reversing the progression of the disorder underlying such symptoms, (iii) reducing or eliminating the likelihood of the symptoms' recurrence, and/or (iv) slowing the progression of, lowering or eliminating the disorder. In the preferred embodiment, treating a subject afflicted with a disorder includes (i) reversing the progression of one or more of the disorder's symptoms, (ii) reversing the progression of the disorder underlying such symptoms, (iii) preventing the symptoms' recurrence, and/or (iv) eliminating the disorder. For a subject infected with SARS-CoV-2 but not symptomatic of that infection, “treating” the subject also includes, without limitation, reducing the likelihood of the subject's becoming symptomatic of the infection, and preferably, preventing the subject from becoming symptomatic of the infection.

### Embodiments of the Invention

This invention provides certain bispecific antibodies that bind both to hACE2 and TMPRSS2. It also provides recombinant viral particles (preferably recombinant AAV particles) that, when introduced into a subject, cause the long-term expression of those antibodies. These antibodies and viral particles permit prophylaxis and therapy for SARS-CoV-2 infection. Supporting this approach is the recently published reference of Du, et al., which provides in vivo evidence that an anti-hACE2 monoclonal antibody can be used to prevent and treat SARS-CoV-2 infection.

Specifically, this invention provides a bispecific antibody that (i) specifically binds to the extracellular portion of human angiotensin converting enzyme 2 (hACE2); (ii) specifically binds to the extracellular portion of human TMPRSS2 (hTMPRSS2); (iii) does not significantly inhibit the ability of hACE2 to cleave angiotensin II and/or a synthetic MCA-based peptide; and (iv) specifically inhibits the entry into hACE2⁺/hTMPRSS2⁺ human cells of a pseudovirus bearing SARS-CoV-2 S protein.

This invention also provides a bispecific antibody that (i) specifically binds to the extracellular portion of human angiotensin converting enzyme 2 (hACE2); (ii) specifically binds to the extracellular portion of human TMPRSS2 (hTMPRSS2); (iii) does not significantly inhibit the ability of hACE2 to cleave angiotensin II and/or a synthetic MCA-based peptide; (iv) specifically inhibits the entry into hACE2⁺/hTMPRSS2⁺ human cells of a pseudovirus bearing SARS-CoV-2 S protein; and (v) specifically inhibits binding of SARS-CoV-2 (and/or the SARS-CoV-2 S1 protein receptor binding domain fragment (e.g., the protein consisting of S amino acid residues 331 to 524)) to the extracellular portion of hACE2.

This invention further provides a bispecific antibody that (i) specifically binds to the extracellular portion of human angiotensin converting enzyme 2 (hACE2); (ii) specifically binds to the extracellular portion of human TMPRSS2 (hTMPRSS2); (iii) does not significantly inhibit the ability of hACE2 to cleave angiotensin II and/or a synthetic MCA-based peptide; (iv) specifically inhibits the entry into hACE2⁺/hTMPRSS2⁺ human cells of a pseudovirus bearing SARS-CoV-2 S protein; and (v) specifically inhibits the entry of SARS-CoV-2 into hACE2⁺/hTMPRSS2⁺ human cells.

This invention further provides a bispecific antibody that (i) specifically binds to the extracellular portion of human angiotensin converting enzyme 2 (hACE2); (ii) specifically binds to the extracellular portion of human TMPRSS2 (hTMPRSS2); (iii) does not significantly inhibit the ability of hACE2 to cleave angiotensin II and/or a synthetic MCA-based peptide; (iv) specifically inhibits the entry into hACE2⁺/hTMPRSS2⁺ human cells of a pseudovirus bearing SARS-CoV-2 S protein; and (v) specifically inhibits cleavage of SARS-CoV-2 S protein by hTMPRSS2.

This invention further provides a bispecific antibody that (i) specifically binds to the extracellular portion of human angiotensin converting enzyme 2 (hACE2); (ii) specifically binds to the extracellular portion of human TMPRSS2 (hTMPRSS2); (iii) does not significantly inhibit the ability of hACE2 to cleave angiotensin II and/or a synthetic MCA-based peptide; (iv) specifically inhibits the entry into hACE2⁺/hTMPRSS2⁺ human cells of a pseudovirus bearing SARS-CoV-2 S protein; (v) specifically inhibits binding of SARS-CoV-2 (and/or the SARS-CoV-2 S1 protein receptor binding domain fragment (e.g., the protein consisting of S amino acid residues 331 to 524)) to the extracellular portion of hACE2; and (vi) specifically inhibits the entry of SARS-CoV-2 into hACE2⁺/hTMPRSS2⁺ human cells.

This invention further provides a bispecific antibody that (i) specifically binds to the extracellular portion of human angiotensin converting enzyme 2 (hACE2); (ii) specifically binds to the extracellular portion of human TMPRSS2 (hTMPRSS2); (iii) does not significantly inhibit the ability of hACE2 to cleave angiotensin II and/or a synthetic MCA-based peptide; (iv) specifically inhibits the entry into hACE2⁺/hTMPRSS2⁺ human cells of a pseudovirus bearing SARS-CoV-2 S protein; (v) specifically inhibits the entry of SARS-CoV-2 into hACE2⁺/hTMPRSS2⁺ human cells; and (vi) specifically inhibits cleavage of SARS-CoV-2 S protein by hTMPRSS2.

This invention further provides a bispecific antibody that (i) specifically binds to the extracellular portion of human angiotensin converting enzyme 2 (hACE2); (ii) specifically binds to the extracellular portion of human TMPRSS2 (hTMPRSS2); (iii) does not significantly inhibit the ability of hACE2 to cleave angiotensin II and/or a synthetic MCA-based peptide; (iv) specifically inhibits the entry into hACE2⁺/hTMPRSS2⁺ human cells of a pseudovirus bearing SARS-CoV-2 S protein; (v) specifically inhibits binding of SARS-CoV-2 (and/or the SARS-CoV-2 S1 protein receptor binding domain fragment (e.g., the protein consisting of S amino acid residues 331 to 524)) to the extracellular portion of hACE2; and (vi) specifically inhibits cleavage of SARS-CoV-2 S protein by hTMPRSS2.

This invention still further provides a bispecific antibody that (i) specifically binds to the extracellular portion of human angiotensin converting enzyme 2 (hACE2); (ii) specifically binds to the extracellular portion of human TMPRSS2 (hTMPRSS2); (iii) does not significantly inhibit the ability of hACE2 to cleave angiotensin II and/or a synthetic MCA-based peptide; (iv) specifically inhibits the entry into hACE2⁺/hTMPRSS2⁺ human cells of a pseudovirus bearing SARS-CoV-2 S protein; (v) specifically inhibits binding of SARS-CoV-2 (and/or the SARS-CoV-2 S1 protein receptor binding domain fragment (e.g., the protein consisting of S amino acid residues 331 to 524)) to the extracellular portion of hACE2; (vi) specifically inhibits the entry of SARS-CoV-2 into hACE2⁺/hTMPRSS2⁺ human cells; and (vii) specifically inhibits cleavage of SARS-CoV-2 S protein by hTMPRSS2.

The above eight bispecific antibodies are referred to herein, collectively and individually, as the present bispecific antibody. SARS-CoV-2 pseudoviruses and methods of making and using them are known, as are SARS-CoV-2 S1 protein receptor binding domain (RBD) fragments. See, e.g., Shang, et al., and Hoffman, et al. (Cell 2020).

In a preferred embodiment, the present bispecific antibody does not significantly inhibit the ability of hACE2 to cleave angiotensin II (i.e., to convert angiotensin II to angiotensin-(1-7). This inhibition can be measured according to the methods in the examples section below. A specific example of this embodiment of the invention is an antibody that (i) binds to the extracellular portion of hACE2 with an affinity of 50 nM; (ii) reduces binding of SARS-CoV-2 to the extracellular portion of hACE2 by a factor of 100,000; and (iii) inhibits by 20% the ability of hACE2 to cleave angiotensin

In a second embodiment, the present bispecific antibody does not significantly inhibit the ability of hACE2 to cleave des-Arg-bradykinin. This inhibition can be measured according to the methods in the examples section below. A specific example of this embodiment of the invention is an antibody that (i) binds to the extracellular portion of hACE2 with an affinity of 50 nM; (ii) reduces binding of SARS-CoV-2 to the extracellular portion of hACE2 by a factor of 100,000; and (iii) inhibits by 20% the ability of hACE2 to cleave des-Arg-bradykinin.

In a third embodiment, the present bispecific antibody does not significantly inhibit the ability of hACE2 to cleave neurotensin. This inhibition can be measured according to the methods in the examples section below. A specific example of this embodiment of the invention is an antibody that (i) binds to the extracellular portion of hACE2 with an affinity of 50 nM; (ii) reduces binding of SARS-CoV-2 to the extracellular portion of hACE2 by a factor of 100,000; and (iii) inhibits by 20% the ability of hACE2 to cleave neurotensin.

In a fourth embodiment, the present bispecific antibody does not significantly inhibit the ability of hACE2 to cleave kinetensin. This inhibition can be measured according to the methods in the examples section below. A specific example of this embodiment of the invention is an antibody that (i) binds to the extracellular portion of hACE2 with an affinity of 50 nM; (ii) reduces binding of SARS-CoV-2 to the extracellular portion of hACE2 by a factor of 100,000; and (iii) inhibits by 20% the ability of hACE2 to cleave kinetensin.

In a fifth embodiment, the present bispecific antibody does not significantly inhibit the ability of hACE2 to cleave a synthetic MCA-based peptide. This inhibition can be measured according to the methods in the examples section below. A specific example of this embodiment of the invention is an antibody that (i) binds to the extracellular portion of hACE2 with an affinity of 50 nM; (ii) reduces binding of SARS-CoV-2 to the extracellular portion of hACE2 by a factor of 100,000; and (iii) inhibits by 20% the ability of hACE2 to cleave a synthetic MCA-based peptide (preferably Mca-APK(Dnp), which is also referred to as Mac-APK-Dnp). As shown in the examples below, these peptides can be used to measure the inhibition of hACE2 carboxypeptidase activity.

In a sixth embodiment, the present bispecific antibody does not significantly inhibit the ability of hACE2 to cleave apelin-13. This inhibition can be measured according to the methods in the examples section below. A specific example of this embodiment of the invention is an antibody that (i) binds to the extracellular portion of hACE2 with an affinity of 50 nM; (ii) reduces binding of SARS-CoV-2 to the extracellular portion of hACE2 by a factor of 100,000; and (iii) inhibits by 20% the ability of hACE2 to cleave apelin-13.

In a seventh embodiment, the present bispecific antibody does not significantly inhibit the ability of hACE2 to cleave dynorphin A 1-13. This inhibition can be measured according to the methods in the examples section below. A specific example of this embodiment of the invention is an antibody that (i) binds to the extracellular portion of hACE2 with an affinity of 50 nM; (ii) reduces binding of SARS-CoV-2 to the extracellular portion of hACE2 by a factor of 100,000; and (iii) inhibits by 20% the ability of hACE2 to cleave dynorphin A 1-13.

In another preferred embodiment of the invention, the present bispecific antibody binds to an epitope that does not include hACE2 amino acid residues required for normal function. So, in one embodiment, the present bispecific antibody does not specifically bind to an epitope on hACE2 comprising an amino acid residue selected from the group consisting of Arg273, His345, Pro346, His374, Glu375, His378, Glu402, His505, and Tyr515. The following embodiments are exemplary. (i) The present bispecific antibody does not specifically bind to an epitope on hACE2 comprising Arg273. (ii) The present bispecific antibody does not specifically bind to an epitope on hACE2 comprising His345. (iii) The present bispecific antibody does not specifically bind to an epitope on hACE2 comprising Pro346. (iv) The present bispecific antibody does not specifically bind to an epitope on hACE2 comprising His374. (v) The present bispecific antibody does not specifically bind to an epitope on hACE2 comprising Glu375. (vi) The present bispecific antibody does not specifically bind to an epitope on hACE2 comprising His378. (vii) The present bispecific antibody does not specifically bind to an epitope on hACE2 comprising Glu402. (viii) The present bispecific antibody does not specifically bind to an epitope on hACE2 comprising His505. (ix) The present bispecific antibody does not specifically bind to an epitope on hACE2 comprising Tyr515.

In another embodiment, the present bispecific antibody does not specifically bind to an epitope on hACE2 comprising an amino acid residue selected from the group consisting of residues 19 to 102, residues 290 to 397, and residues 417 to 430. The following embodiments are exemplary. (i) The present bispecific antibody does not specifically bind to an epitope on hACE2 comprising an amino acid residue within residues 19 to 102. (ii) The present bispecific antibody does not specifically bind to an epitope on hACE2 comprising an amino acid residue within residues 290 to 397. (iii) The present bispecific antibody does not specifically bind to an epitope on hACE2 comprising an amino acid residue within residues 417 to 430.

In a further embodiment, the present bispecific antibody does not specifically bind to an epitope on hACE2 comprising an amino acid residue selected from the group consisting of residues 103 to 289, residues 398 to 416, and residues 431 to 615. The following embodiments are exemplary. (i) The present bispecific antibody does not specifically bind to an epitope on hACE2 comprising an amino acid residue within residues 103 to 289. (ii) The present bispecific antibody does not specifically bind to an epitope on hACE2 comprising an amino acid residue within residues 398 to 416. (iii) The present bispecific antibody does not specifically bind to an epitope on hACE2 comprising an amino acid residue within residues 431 to 615.

In a further embodiment, the present bispecific antibody specifically binds to an epitope on hACE2 comprising an amino acid residue selected from the group consisting of residues 1-18, residues 417-430, and residues 616-740. The following embodiments are exemplary. (i) The present bispecific antibody specifically binds to an epitope on hACE2 comprising an amino acid residue within residues 1-5. (ii) The present bispecific antibody specifically binds to an epitope on hACE2 comprising an amino acid residue within residues 5-10. (iii) The present bispecific antibody specifically binds to an epitope on hACE2 comprising an amino acid residue within residues 10-15. (iv) The present bispecific antibody specifically binds to an epitope on hACE2 comprising an amino acid residue within residues 15-18. (v) The present bispecific antibody specifically binds to an epitope on hACE2 comprising an amino acid residue within residues 417-420. (vi) The present bispecific antibody specifically binds to an epitope on hACE2 comprising an amino acid residue within residues 420-425. (vii) The present bispecific antibody specifically binds to an epitope on hACE2 comprising an amino acid residue within residues 425-430. (viii) The present bispecific antibody specifically binds to an epitope on hACE2 comprising an amino acid residue within residues 616-620. (ix) The present bispecific antibody specifically binds to an epitope on hACE2 comprising an amino acid residue within residues 620-625. (x) The present bispecific antibody specifically binds to an epitope on hACE2 comprising an amino acid residue within residues 625-630. (xi) The present bispecific antibody specifically binds to an epitope on hACE2 comprising an amino acid residue within residues 630-635. (xii) The present bispecific antibody specifically binds to an epitope on hACE2 comprising an amino acid residue within residues 635-640. (xiii) The present bispecific antibody specifically binds to an epitope on hACE2 comprising an amino acid residue within residues 640-645. (xiv) The present bispecific antibody specifically binds to an epitope on hACE2 comprising an amino acid residue within residues 645-650. (xv) The present bispecific antibody specifically binds to an epitope on hACE2 comprising an amino acid residue within residues 650-655. (xvi) The present bispecific antibody specifically binds to an epitope on hACE2 comprising an amino acid residue within residues 655-660. (xvii) The present bispecific antibody specifically binds to an epitope on hACE2 comprising an amino acid residue within residues 660-665. (xviii) The present bispecific antibody specifically binds to an epitope on hACE2 comprising an amino acid residue within residues 665-670. (xix) The present bispecific antibody specifically binds to an epitope on hACE2 comprising an amino acid residue within residues 670-675. (xx) The present bispecific antibody specifically binds to an epitope on hACE2 comprising an amino acid residue within residues 675-680. (xxi) The present bispecific antibody specifically binds to an epitope on hACE2 comprising an amino acid residue within residues 680-685. (xxii) The present bispecific antibody specifically binds to an epitope on hACE2 comprising an amino acid residue within residues 685-690. (xxiii) The present bispecific antibody specifically binds to an epitope on hACE2 comprising an amino acid residue within residues 690-695. (xxiv) The present bispecific antibody specifically binds to an epitope on hACE2 comprising an amino acid residue within residues 695-700. (xxv) The present bispecific antibody specifically binds to an epitope on hACE2 comprising an amino acid residue within residues 700-705. (xxvi) The present bispecific antibody specifically binds to an epitope on hACE2 comprising an amino acid residue within residues 705-710. (xxvii) The present bispecific antibody specifically binds to an epitope on hACE2 comprising an amino acid residue within residues 710-715. (xviii) The present bispecific antibody specifically binds to an epitope on hACE2 comprising an amino acid residue within residues 715-720. (xxix) The present bispecific antibody specifically binds to an epitope on hACE2 comprising an amino acid residue within residues 720-725. (xxx)

The present bispecific antibody specifically binds to an epitope on hACE2 comprising an amino acid residue within residues 725-730. (xxxi) The present bispecific antibody specifically binds to an epitope on hACE2 comprising an amino acid residue within residues 730-735. (xxxii) The present bispecific antibody specifically binds to an epitope on hACE2 comprising an amino acid residue within residues 735-740.

In a further embodiment, the present bispecific antibody specifically binds to an epitope on hACE2 comprising an amino acid residue selected from the group consisting of residues 19-416. The following embodiments are exemplary. (i) The present bispecific antibody specifically binds to an epitope on hACE2 comprising an amino acid residue within residues 19-25. (ii) The present bispecific antibody specifically binds to an epitope on hACE2 comprising an amino acid residue within residues 26-30. (iii) The present bispecific antibody specifically binds to an epitope on hACE2 comprising an amino acid residue within residues 31-35. (iv) The present bispecific antibody specifically binds to an epitope on hACE2 comprising an amino acid residue within residues 36-40. (v) The present bispecific antibody specifically binds to an epitope on hACE2 comprising an amino acid residue within residues 41-45. (vi) The present bispecific antibody specifically binds to an epitope on hACE2 comprising an amino acid residue within residues 46-50. (vii) The present bispecific antibody specifically binds to an epitope on hACE2 comprising an amino acid residue within residues 51-55. (viii) The present bispecific antibody specifically binds to an epitope on hACE2 comprising an amino acid residue within residues 56-60. (ix) The present bispecific antibody specifically binds to an epitope on hACE2 comprising an amino acid residue within residues 61-65. (x) The present bispecific antibody specifically binds to an epitope on hACE2 comprising an amino acid residue within residues 66-70. (xi) The present bispecific antibody specifically binds to an epitope on hACE2 comprising an amino acid residue within residues 71-75. (xii) The present bispecific antibody specifically binds to an epitope on hACE2 comprising an amino acid residue within residues 76-80. (xiii) The present bispecific antibody specifically binds to an epitope on hACE2 comprising an amino acid residue within residues 81-85. (xiv) The present bispecific antibody specifically binds to an epitope on hACE2 comprising an amino acid residue within residues 86-90. (xv) The present bispecific antibody specifically binds to an epitope on hACE2 comprising an amino acid residue within residues 91-95. (xvi) The present bispecific antibody specifically binds to an epitope on hACE2 comprising an amino acid residue within residues 96-100. (xvii) The present bispecific antibody specifically binds to an epitope on hACE2 comprising an amino acid residue within residues 101-105. (xviii) The present bispecific antibody specifically binds to an epitope on hACE2 comprising an amino acid residue within residues 106-110. (xix) The present bispecific antibody specifically binds to an epitope on hACE2 comprising an amino acid residue within residues 111-115. (xx) The present bispecific antibody specifically binds to an epitope on hACE2 comprising an amino acid residue within residues 116-120. (xxi) The present bispecific antibody specifically binds to an epitope on hACE2 comprising an amino acid residue within residues 121-125. (xxii) The present bispecific antibody specifically binds to an epitope on hACE2 comprising an amino acid residue within residues 126-130. (xxiii) The present bispecific antibody specifically binds to an epitope on hACE2 comprising an amino acid residue within residues 131-135. (xxiv) The present bispecific antibody specifically binds to an epitope on hACE2 comprising an amino acid residue within residues 136-140. (xxv) The present bispecific antibody specifically binds to an epitope on hACE2 comprising an amino acid residue within residues 141-145. (xxvi) The present bispecific antibody specifically binds to an epitope on hACE2 comprising an amino acid residue within residues 146-150. (xxvii) The present bispecific antibody specifically binds to an epitope on hACE2 comprising an amino acid residue within residues 151-155. (xxviii) The present bispecific antibody specifically binds to an epitope on hACE2 comprising an amino acid residue within residues 156-160. (xxix) The present bispecific antibody specifically binds to an epitope on hACE2 comprising an amino acid residue within residues 161-165. (xxx) The present bispecific antibody specifically binds to an epitope on hACE2 comprising an amino acid residue within residues 166-170. (xxxi) The present bispecific antibody specifically binds to an epitope on hACE2 comprising an amino acid residue within residues 171-175. (xxxii) The present bispecific antibody specifically binds to an epitope on hACE2 comprising an amino acid residue within residues 176-180. (xxxiii) The present bispecific antibody specifically binds to an epitope on hACE2 comprising an amino acid residue within residues 181-185. (xxxiv) The present bispecific antibody specifically binds to an epitope on hACE2 comprising an amino acid residue within residues 186-190. (xxxv) The present bispecific antibody specifically binds to an epitope on hACE2 comprising an amino acid residue within residues 191-195. (xxxvi) The present bispecific antibody specifically binds to an epitope on hACE2 comprising an amino acid residue within residues 196-200. (xxxvii) The present bispecific antibody specifically binds to an epitope on hACE2 comprising an amino acid residue within residues 201-205. (xxxviii) The present bispecific antibody specifically binds to an epitope on hACE2 comprising an amino acid residue within residues 206-210. (xxxix) The present bispecific antibody specifically binds to an epitope on hACE2 comprising an amino acid residue within residues 211-215. (xl) The present bispecific antibody specifically binds to an epitope on hACE2 comprising an amino acid residue within residues 216-220. (xli) The present bispecific antibody specifically binds to an epitope on hACE2 comprising an amino acid residue within residues 221-225. (xlii) The present bispecific antibody specifically binds to an epitope on hACE2 comprising an amino acid residue within residues 226-230. (xliii) The present bispecific antibody specifically binds to an epitope on hACE2 comprising an amino acid residue within residues 231-235. (xliv) The present bispecific antibody specifically binds to an epitope on hACE2 comprising an amino acid residue within residues 236-240. (xlv) The present bispecific antibody specifically binds to an epitope on hACE2 comprising an amino acid residue within residues 241-245. (xlvi) The present bispecific antibody specifically binds to an epitope on hACE2 comprising an amino acid residue within residues 246-250. (xlvii) The present bispecific antibody specifically binds to an epitope on hACE2 comprising an amino acid residue within residues 251-255. (xlviii) The present bispecific antibody specifically binds to an epitope on hACE2 comprising an amino acid residue within residues 256-260. (xlix) The present bispecific antibody specifically binds to an epitope on hACE2 comprising an amino acid residue within residues 261-265. (l) The present bispecific antibody specifically binds to an epitope on hACE2 comprising an amino acid residue within residues 266-270. (li) The present bispecific antibody specifically binds to an epitope on hACE2 comprising an amino acid residue within residues 271-275. (lii) The present bispecific antibody specifically binds to an epitope on hACE2 comprising an amino acid residue within residues 276-280. (liii) The present bispecific antibody specifically binds to an epitope on hACE2 comprising an amino acid residue within residues 281-285. (liv) The present bispecific antibody specifically binds to an epitope on hACE2 comprising an amino acid residue within residues 286-290. (lv) The present bispecific antibody specifically binds to an epitope on hACE2 comprising an amino acid residue within residues 291-295. (lvi) The present bispecific antibody specifically binds to an epitope on hACE2 comprising an amino acid residue within residues 296-300. (lvii) The present bispecific antibody specifically binds to an epitope on hACE2 comprising an amino acid residue within residues 301-305. (lviii) The present bispecific antibody specifically binds to an epitope on hACE2 comprising an amino acid residue within residues 306-310. (lix) The present bispecific antibody specifically binds to an epitope on hACE2 comprising an amino acid residue within residues 311-315. (lx) The present bispecific antibody specifically binds to an epitope on hACE2 comprising an amino acid residue within residues 316-320. (lxi) The present bispecific antibody specifically binds to an epitope on hACE2 comprising an amino acid residue within residues 321-325. (lxii) The present bispecific antibody specifically binds to an epitope on hACE2 comprising an amino acid residue within residues 326-330. (lxiii) The present bispecific antibody specifically binds to an epitope on hACE2 comprising an amino acid residue within residues 331-335. (lxiv) The present bispecific antibody specifically binds to an epitope on hACE2 comprising an amino acid residue within residues 336-340. (lxv) The present bispecific antibody specifically binds to an epitope on hACE2 comprising an amino acid residue within residues 341-345. (lxvi) The present bispecific antibody specifically binds to an epitope on hACE2 comprising an amino acid residue within residues 346-350. (lxvii) The present bispecific antibody specifically binds to an epitope on hACE2 comprising an amino acid residue within residues 351-355. (lxviii) The present bispecific antibody specifically binds to an epitope on hACE2 comprising an amino acid residue within residues 356-360. (lxix) The present bispecific antibody specifically binds to an epitope on hACE2 comprising an amino acid residue within residues 361-365. (lxx) The present bispecific antibody specifically binds to an epitope on hACE2 comprising an amino acid residue within residues 366-370. (lxxi) The present bispecific antibody specifically binds to an epitope on hACE2 comprising an amino acid residue within residues 371-375. (lxxii) The present bispecific antibody specifically binds to an epitope on hACE2 comprising an amino acid residue within residues 376-380. (lxxiii) The present bispecific antibody specifically binds to an epitope on hACE2 comprising an amino acid residue within residues 381-385. (lxxiv) The present bispecific antibody specifically binds to an epitope on hACE2 comprising an amino acid residue within residues 386-390. (lxxv) The present bispecific antibody specifically binds to an epitope on hACE2 comprising an amino acid residue within residues 391-395. (lxxvi) The present bispecific antibody specifically binds to an epitope on hACE2 comprising an amino acid residue within residues 396-400. (lxxvii) The present bispecific antibody specifically binds to an epitope on hACE2 comprising an amino acid residue within residues 401-405. (lxxviii) The present bispecific antibody specifically binds to an epitope on hACE2 comprising an amino acid residue within residues 406-410. (lxxix) The present bispecific antibody specifically binds to an epitope on hACE2 comprising an amino acid residue within residues 411-416.

In a further embodiment, the present bispecific antibody specifically binds to an epitope on hACE2 comprising an amino acid residue selected from the group consisting of residues 431-615. The following embodiments are exemplary. (i) The present bispecific antibody specifically binds to an epitope on hACE2 comprising an amino acid residue within residues 431-435. (ii) The present bispecific antibody specifically binds to an epitope on hACE2 comprising an amino acid residue within residues 436-440. (iii) The present bispecific antibody specifically binds to an epitope on hACE2 comprising an amino acid residue within residues 441-445. (iv) The present bispecific antibody specifically binds to an epitope on hACE2 comprising an amino acid residue within residues 446-450. (v) The present bispecific antibody specifically binds to an epitope on hACE2 comprising an amino acid residue within residues 451-455. (vi) The present bispecific antibody specifically binds to an epitope on hACE2 comprising an amino acid residue within residues 456-460. (vii) The present bispecific antibody specifically binds to an epitope on hACE2 comprising an amino acid residue within residues 461-465. (viii) The present bispecific antibody specifically binds to an epitope on hACE2 comprising an amino acid residue within residues 466-470. (ix) The present bispecific antibody specifically binds to an epitope on hACE2 comprising an amino acid residue within residues 471-475. (x) The present bispecific antibody specifically binds to an epitope on hACE2 comprising an amino acid residue within residues 476-480. (xi) The present bispecific antibody specifically binds to an epitope on hACE2 comprising an amino acid residue within residues 481-485. (xii) The present bispecific antibody specifically binds to an epitope on hACE2 comprising an amino acid residue within residues 486-490. (xiii) The present bispecific antibody specifically binds to an epitope on hACE2 comprising an amino acid residue within residues 491-495. (xiv) The present bispecific antibody specifically binds to an epitope on hACE2 comprising an amino acid residue within residues 496-500. (xv) The present bispecific antibody specifically binds to an epitope on hACE2 comprising an amino acid residue within residues 501-505. (xvi) The present bispecific antibody specifically binds to an epitope on hACE2 comprising an amino acid residue within residues 506-510. (xvii) The present bispecific antibody specifically binds to an epitope on hACE2 comprising an amino acid residue within residues 511-515. (xviii) The present bispecific antibody specifically binds to an epitope on hACE2 comprising an amino acid residue within residues 516-520. (xix) The present bispecific antibody specifically binds to an epitope on hACE2 comprising an amino acid residue within residues 521-525. (xx) The present bispecific antibody specifically binds to an epitope on hACE2 comprising an amino acid residue within residues 526-530. (xxi) The present bispecific antibody specifically binds to an epitope on hACE2 comprising an amino acid residue within residues 531-535. (xxii) The present bispecific antibody specifically binds to an epitope on hACE2 comprising an amino acid residue within residues 536-540. (xxiii) The present bispecific antibody specifically binds to an epitope on hACE2 comprising an amino acid residue within residues 541-545. (xxiv) The present bispecific antibody specifically binds to an epitope on hACE2 comprising an amino acid residue within residues 546-550. (xxv) The present bispecific antibody specifically binds to an epitope on hACE2 comprising an amino acid residue within residues 551-555. (xxvi) The present bispecific antibody specifically binds to an epitope on hACE2 comprising an amino acid residue within residues 556-560. (xxvii) The present bispecific antibody specifically binds to an epitope on hACE2 comprising an amino acid residue within residues 561-565. (xxviii) The present bispecific antibody specifically binds to an epitope on hACE2 comprising an amino acid residue within residues 566-570. (xxix) The present bispecific antibody specifically binds to an epitope on hACE2 comprising an amino acid residue within residues 571-575. (xxx) The present bispecific antibody specifically binds to an epitope on hACE2 comprising an amino acid residue within residues 576-580. (xxxi) The present bispecific antibody specifically binds to an epitope on hACE2 comprising an amino acid residue within residues 581-585. (xxxii) The present bispecific antibody specifically binds to an epitope on hACE2 comprising an amino acid residue within residues 586-590. (xxxiii) The present bispecific antibody specifically binds to an epitope on hACE2 comprising an amino acid residue within residues 591-595. (xxxiv) The present bispecific antibody specifically binds to an epitope on hACE2 comprising an amino acid residue within residues 596-600. (xxxv) The present bispecific antibody specifically binds to an epitope on hACE2 comprising an amino acid residue within residues 601-605. (xxxvi) The present bispecific antibody specifically binds to an epitope on hACE2 comprising an amino acid residue within residues 606-610. (xxxvii) The present bispecific antibody specifically binds to an epitope on hACE2 comprising an amino acid residue within residues 611-615.

In a further embodiment, the present bispecific antibody specifically binds to an epitope on hACE2 comprising an amino acid residue selected from the group consisting of Ser19, Gln24, Thr27, Phe28, Lys31, His34, Glu35, Glu37, Asp38, Tyr41, Gln42, Leu45, Leu79, Met82, Tyr83, Gln325, Glu329, Asn330, Lys353, Gly354, Asp355, and Arg357. The following embodiments are exemplary. (i) The present bispecific antibody specifically binds to an epitope on hACE2 comprising residue Ser19. (ii) The present bispecific antibody specifically binds to an epitope on hACE2 comprising residue Gln24. (iii) The present bispecific antibody specifically binds to an epitope on hACE2 comprising residue Thr27. (iv) The present bispecific antibody specifically binds to an epitope on hACE2 comprising residue Phe28. (v) The present bispecific antibody specifically binds to an epitope on hACE2 comprising residue Lys31. (vi) The present bispecific antibody specifically binds to an epitope on hACE2 comprising residue His34. (vii) The present bispecific antibody specifically binds to an epitope on hACE2 comprising residue Glu35. (viii) The present bispecific antibody specifically binds to an epitope on hACE2 comprising residue Glu37. (ix) The present bispecific antibody specifically binds to an epitope on hACE2 comprising residue Asp38. (x) The present bispecific antibody specifically binds to an epitope on hACE2 comprising residue Tyr41. (xi) The present bispecific antibody specifically binds to an epitope on hACE2 comprising residue Gln42. (xii) The present bispecific antibody specifically binds to an epitope on hACE2 comprising residue Leu45. (xiii) The present bispecific antibody specifically binds to an epitope on hACE2 comprising residue Leu79. (xiv) The present bispecific antibody specifically binds to an epitope on hACE2 comprising residue Met82. (xv) The present bispecific antibody specifically binds to an epitope on hACE2 comprising residue Tyr83. (xvi) The present bispecific antibody specifically binds to an epitope on hACE2 comprising residue Gln325. (xvii) The present bispecific antibody specifically binds to an epitope on hACE2 comprising residue Glu329. (xviii) The present bispecific antibody specifically binds to an epitope on hACE2 comprising residue Asn330. (xix) The present bispecific antibody specifically binds to an epitope on hACE2 comprising residue Lys353. (xx) The present bispecific antibody specifically binds to an epitope on hACE2 comprising residue Gly354. (xxi) The present bispecific antibody specifically binds to an epitope on hACE2 comprising residue Asp355. (xxii) The present bispecific antibody specifically binds to an epitope on hACE2 comprising residue Arg357. In a preferred embodiment, the present bispecific antibody specifically binds to an epitope on hACE2 comprising residue Lys31. In another preferred embodiment, the present bispecific antibody specifically binds to an epitope on hACE2 comprising residue Lys353.

Preferably, the present bispecific antibody comprises the heavy and light chain variable regions identified, respectively, as humanized 11B11 VH and humanized 11B11 VK, and set forth in FIG. 5 below (taken from Supplementary FIG. 2 of Du, et al.). These variable regions include heavy chain CDR1 (GFTFIDYYMN), CDR2 (FIRNKANDYTTEYST), and CDR3 (RHMYDDGFDF), and light chain CDR1 (ASSSVRYMH), CDR2 (LLIYDTSKLA), and CDR3 (QQWSYNPLTF). In a preferred embodiment, the present bispecific antibody comprises the heavy chain CDR1 having the amino acid sequence GFTFIDYYMN. In another preferred embodiment, the present bispecific antibody comprises the heavy chain CDR2 having the amino acid sequence FIRNKANDYTTEYST. In another preferred embodiment, the present bispecific antibody comprises the heavy chain CDR3 having the amino acid sequence RHMYDDGFDF. In another preferred embodiment, the present bispecific antibody comprises the light chain CDR1 having the amino acid sequence ASSSVRYMH. In another preferred embodiment, the present bispecific antibody comprises the light chain CDR2 having the amino acid sequence LLIYDTSKLA. In another preferred embodiment, the present bispecific antibody comprises the light chain CDR3 having the amino acid sequence QQWSYNPLTF. In a further preferred embodiment, the present bispecific antibody comprises the heavy chain CDR1 having the amino acid sequence GFTFIDYYMN, the heavy chain CDR2 having the amino acid sequence FIRNKANDYTTEYST, the heavy chain CDR3 having the amino acid sequence RHMYDDGFDF, the light chain CDR1 having the amino acid sequence ASSSVRYMH, the light chain CDR2 having the amino acid sequence LLIYDTSKLA, and the light chain CDR3 having the amino acid sequence QQWSYNPLTF. The following additional embodiments are envisioned, and are exemplified in Examples 15 and 16 below. (i) The present bispecific antibody comprises a point mutant of the heavy chain CDR1. (ii) The present bispecific antibody comprises a point mutant of the heavy chain CDR2. (iii) The present bispecific antibody comprises a point mutant of the heavy chain CDR3. (iv) The present bispecific antibody comprises a point mutant of the light chain CDR1. (v) The present bispecific antibody comprises a point mutant of the light chain CDR2. (vi) The present bispecific antibody comprises a point mutant of the light chain CDR3.

In yet a further embodiment, the present bispecific antibody comprises a heavy chain CDR3 comprising an amino acid sequence selected from the group consisting of (i) CAKDRGYSSSWYGGFDYW; (ii) CARHTWWKGAGFFDHW; (iii) CARGTRFLEWSLPLDVW; (iv) CATTENPNPRW; (v) CATTEDPYPRW; (vi) CARASPNTGWHFDHW; (vii) CATTMNPNPRW; (viii) CAAIAYEEGVYR-WDW; and (ix) RHMYDDGFDF. The following embodiments are exemplary. (i) The present bispecific antibody comprises a heavy chain CDR3 comprising the amino acid sequence CAKDRGYSSSWYGGFDYW. (ii) The present bispecific antibody comprises a heavy chain CDR3 comprising the amino acid sequence CARHTWWKGAGF-FDHW. (iii) The present bispecific antibody comprises a heavy chain CDR3 comprising the amino acid sequence CARGTRFLEWSLPLDVW. (iv) The present bispecific antibody comprises a heavy chain CDR3 comprising the amino acid sequence CATTENPNPRW. (v) The present bispecific antibody comprises a heavy chain CDR3 comprising the amino acid sequence CATTEDPYPRW. (vi) The present bispecific antibody comprises a heavy chain CDR3 comprising the amino acid sequence CARASPNTGWHFDHW. (vii) The present bispecific antibody comprises a heavy chain CDR3 comprising the amino acid sequence CATTMNPNPRW. (viii) The present bispecific antibody comprises a heavy chain CDR3 comprising the amino acid sequence CAAIAYEEGVYRWDW.

In yet a further embodiment, the present bispecific antibody comprises one or more of (i) a heavy chain CDR1 comprising the amino acid sequence GFTFIDYYMN; (ii) a heavy chain CDR2 comprising the amino acid sequence FIRNKANDYTTEYST; (iii) a heavy chain CDR3 comprising the amino acid sequence RHMYDDGFDF; (iv) a light chain CDR1 comprising the amino acid sequence ASSSVRYMH; (v) a light chain CDR2 comprising the amino acid sequence LLIYDTSKLA; and (vi) a light chain CDR3 comprising the amino acid sequence QQWSYNPLTF. Preferably, the present bispecific antibody comprises (i) a heavy chain CDR1 comprising the amino acid sequence GFTFIDYYMN; (ii) a heavy chain CDR2 comprising the amino acid sequence FIRNKANDYTTEYST; (iii) a heavy chain CDR3 comprising the amino acid sequence RHMYDDGFDF; (iv) a light chain CDR1 comprising the amino acid sequence ASSSVRYMH; (v) a light chain CDR2 comprising the amino acid sequence LLIYDTSKLA; and (vi) a light chain CDR3 comprising the amino acid sequence QQWSYNPLTF.

In one embodiment, the present bispecific antibody does not significantly inhibit the ability of human TMPRSS1 to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS1 to cleave its substrate by 20%.

In a second embodiment, the present bispecific antibody does not significantly inhibit the ability of human TMPRSS3 to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS3 to cleave its substrate by 20%.

In a third embodiment, the present bispecific antibody does not significantly inhibit the ability of human TMPRSS4 to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS4 to cleave its substrate by 20%.

In a fourth embodiment, the present bispecific antibody does not significantly inhibit the ability of human TMPRSS5 to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS5 to cleave its substrate by 20%.

In a fifth embodiment, the present bispecific antibody does not significantly inhibit the ability of human TMPRSS6 to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS6 to cleave its substrate by 20%.

In a sixth embodiment, the present bispecific antibody does not significantly inhibit the ability of human TMPRSS7 to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS7 to cleave its substrate by 20%.

In a seventh embodiment, the present bispecific antibody does not significantly inhibit the ability of human TMPRSS9 to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS9 to cleave its substrate by 20%.

In an eighth embodiment, the present bispecific antibody does not significantly inhibit the ability of human TMPRSS10 to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS10 to cleave its substrate by 20%.

In a ninth embodiment, the present bispecific antibody does not significantly inhibit the ability of human TMPRSS11A to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS11A to cleave its substrate by 20%.

In a tenth embodiment, the present bispecific antibody does not significantly inhibit the ability of human TMPRSS11B to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS11B to cleave its substrate by 20%.

In an eleventh embodiment, the present bispecific antibody does not significantly inhibit the ability of human TMPRSS11C to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS11C to cleave its substrate by 20%.

In a twelfth embodiment, the present bispecific antibody does not significantly inhibit the ability of human TMPRSS11D to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS11D to cleave its substrate by 20%.

In a thirteenth embodiment, the present bispecific antibody does not significantly inhibit the ability of human TMPRSS11E to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS11E to cleave its substrate by 20%.

In a fourteenth embodiment, the present bispecific antibody does not significantly inhibit the ability of human TMPRSS11F to cleave its substrate. This inhibition can be measured according to the methods in the examples section below. A specific example of this embodiment of the invention is an antibody that (i) binds to the extracellular portion of hTMPRSS2 with an affinity of 50 nM; (ii) reduces the entry into hACE2⁺/hTMPRSS2⁺ human cells of a pseudovirus bearing SARS-CoV-2 S protein by a factor of 10,000; and (iii) reduces the ability of human TMPRSS11F to cleave its substrate by 20%.

In one embodiment, the present bispecific antibody specifically binds to an epitope on hTMPRSS2 comprising amino acid residues in the low-density lipoprotein receptor class A (LDLA) domain. In an exemplary embodiment, the present bispecific antibody specifically binds to an epitope on the LDLA domain comprising an amino acid residue within residues selected from the group consisting of 113-115; 115-120; 120-125; 125-130; 130-135; 135-140; 140-145; and 145-148.

In another embodiment, the present bispecific antibody specifically binds to an epitope on hTMPRSS2 comprising amino acid residues in the scavenger receptor cysteine-rich (SRCR) domain. In an exemplary embodiment, the present bispecific antibody specifically binds to an epitope on the SRCR domain comprising an amino acid residue within residues selected from the group consisting of 149-155; 155-160; 160-165; 165-170; 170-175; 175-180; 180-185; 185-190; 190-195; 195-200; 200-205; 205-210; 210-215; 215-220; 220-225; 225-230; 230-235; and 235-242.

In a further embodiment, the present bispecific antibody specifically binds to an epitope on hTMPRSS2 comprising amino acid residues in the serine protease domain. In an exemplary embodiment, the present bispecific antibody specifically binds to an epitope on the serine protease domain comprising an amino acid residue within residues selected from the group consisting of 255-260; 260-265; 265-270; 270-275; 275-280; 280-285; 285-290; 290-295; 295-300; 300-305; 305-310; 310-315; 315-320; 320-325; 325-330; 330-335; 335-340; 340-345; 345-350; 350-355; 355-360; 360-365; 365-370; 370-375; 375-380; 380-385; 385-390; 390-395; 395-400; 400-405; 405-410; 410-415; 415-420; 420-425; 425-430; 430-435; 435-440; 440-445; 445-450; 450-455; 455-460; 460-465; 465-470; 470-475; 475-480; 480-485; 485-490; and 490-492.

In a further embodiment, the present bispecific antibody specifically binds to an epitope on hTMPRSS2 comprising amino acid residues in the serine protease domain and the SRCR domain. In an exemplary embodiment, the present bispecific antibody specifically binds to an epitope on the serine protease domain and the SRCR domain comprising an amino acid residue within residues selected from the group consisting of 230-270; 230-255; 231-256; 232-257; 233-258; 234-259; 235-260; 236-261; 237-262; 238-263; 239-264; 240-265; 241-266; 242-267; 230-258; 231-259; 232-260; 233-261; 234-262; 235-263; 236-264; 237-265; 238-266; 239-267; 240-268; 241-269; and 242-270.

In yet a further embodiment, the present bispecific antibody specifically binds to an epitope on hTMPRSS2 comprising an amino acid residue within residues selected from the group consisting of 106-200; 200-300; 300-400; 400-492; 106-150; 150-200; 200-250; 250-300; 300-350; 350-400; 400-450; 450-492; 106-110; 110-115; 115-120; 120-125; 125-130; 130-135; 135-140; 140-145; 145-150; 150-155; 155-160; 160-165; 165-170; 170-175; 175-180; 180-185; 185-190; 190-195; 195-200; 200-205; 205-210; 210-215; 215-220; 220-225; 225-230; 230-235; 235-240; 240-245; 245-250; 250-255; 255-260; 260-265; 265-270; 270-275; 275-280; 280-285; 285-290; 290-295; 295-300; 300-305; 305-310; 310-315; 315-320; 320-325; 325-330; 330-335; 335-340; 340-345; 345-350; 350-355; 355-360; 360-365; 365-370; 370-375; 375-380; 380-385; 385-390; 390-395; 395-400; 400-405; 405-410; 410-415; 415-420; 420-425; 425-430; 430-435; 435-440; 440-445; 445-450; 450-455; 455-460; 460-465; 465-470; 470-475; 475-480; 480-485; 485-490; and 490-492.

In a further embodiment, the present bispecific antibody specifically binds to an epitope on hTMPRSS2 comprising an amino acid residue selected from the group consisting of His18, Gln21, Glu23, Asn24, Pro25, Val28, Val49, Pro50, Gln51, Tyr52, Ala53, Pro54, Arg55, Gln59, Val65, Gln68, Pro69, Val96, Gly97, Ala98, Ala99, Ala101, Asn146, Arg147, Cys148, Val149, Arg150, Leu151, Asp187, Met188, Tyr190, Ile221, Tyr222, Lys223, His279, Val280, Cys281, His296, Glu299, Asp345, Asn368, Pro369, Gly370, Met371, Met372, Leu373, Gln374, Glu376, Gln377, Leu378, Asp435, Ser436, Gln438, Asp440, Ser441, Thr447, Lys449, Asn450, Asn451, Ile452, Trp454, Thr459, Ser460, Trp461, Gly464, Val473, and Tyr474. The following embodiments are exemplary. (i) The present bispecific antibody specifically binds to an epitope on hTMPRSS2 comprising residue His18. (ii) The present bispecific antibody specifically binds to an epitope on hTMPRSS2 comprising residue Gln21. (iii) The present bispecific antibody specifically binds to an epitope on hTMPRSS2 comprising residue Glu23. (iv) The present bispecific antibody specifically binds to an epitope on hTMPRSS2 comprising residue Asn24. (v) The present bispecific antibody specifically binds to an epitope on hTMPRSS2 comprising residue Pro25. (vi) The present bispecific antibody specifically binds to an epitope on hTMPRSS2 comprising residue Val28. (vii) The present bispecific antibody specifically binds to an epitope on hTMPRSS2 comprising residue Val49. (viii) The present bispecific antibody specifically binds to an epitope on hTMPRSS2 comprising residue Pro50. (ix) The present bispecific antibody specifically binds to an epitope on hTMPRSS2 comprising residue Gln51. (x) The present bispecific antibody specifically binds to an epitope on hTMPRSS2 comprising residue Tyr52. (xi) The present bispecific antibody specifically binds to an epitope on hTMPRSS2 comprising residue Ala53. (xii) The present bispecific antibody specifically binds to an epitope on hTMPRSS2 comprising residue Pro54. (xiii) The present bispecific antibody specifically binds to an epitope on hTMPRSS2 comprising residue Arg55. (xiv) The present bispecific antibody specifically binds to an epitope on hTMPRSS2 comprising residue Gln59. (xv) The present bispecific antibody specifically binds to an epitope on hTMPRSS2 comprising residue Gln68. (xvi) The present bispecific antibody specifically binds to an epitope on hTMPRSS2 comprising residue Pro69. (xvii) The present bispecific antibody specifically binds to an epitope on hTMPRSS2 comprising residue Val96. (xviii) The present bispecific antibody specifically binds to an epitope on hTMPRSS2 comprising residue Gly97. (xix) The present bispecific antibody specifically binds to an epitope on hTMPRSS2 comprising residue Ala98. (xx) The present bispecific antibody specifically binds to an epitope on hTMPRSS2 comprising residue Ala99. (xxi) The present bispecific antibody specifically binds to an epitope on hTMPRSS2 comprising residue Ala101. (xxii) The present bispecific antibody specifically binds to an epitope on hTMPRSS2 comprising residue Asn146. (xxiii) The present bispecific antibody specifically binds to an epitope on hTMPRSS2 comprising residue Arg147. (xxiv) The present bispecific antibody specifically binds to an epitope on hTMPRSS2 comprising residue Cys148. (xxv) The present bispecific antibody specifically binds to an epitope on hTMPRSS2 comprising residue Val149. (xxvi) The present bispecific antibody specifically binds to an epitope on hTMPRSS2 comprising residue Arg150. (xxvii) The present bispecific antibody specifically binds to an epitope on hTMPRSS2 comprising residue Leu151. (xxviii) The present bispecific antibody specifically binds to an epitope on hTMPRSS2 comprising residue Asp187. (xxix) The present bispecific antibody specifically binds to an epitope on hTMPRSS2 comprising residue Met188. (xxx) The present bispecific antibody specifically binds to an epitope on hTMPRSS2 comprising residue Tyr190. (xxxi) The present bispecific antibody specifically binds to an epitope on hTMPRSS2 comprising residue Ile221. (xxxii) The present bispecific antibody specifically binds to an epitope on hTMPRSS2 comprising residue Tyr222. (xxxiii) The present bispecific antibody specifically binds to an epitope on hTMPRSS2 comprising residue Lys223. (xxxiv) The present bispecific antibody specifically binds to an epitope on hTMPRSS2 comprising residue His279. (xxxv) The present bispecific antibody specifically binds to an epitope on hTMPRSS2 comprising residue Val280. (xxxvi) The present bispecific antibody specifically binds to an epitope on hTMPRSS2 comprising residue Cys281. (xxxvii) The present bispecific antibody specifically binds to an epitope on hTMPRSS2 comprising residue His296. (xxxviii) The present bispecific antibody specifically binds to an epitope on hTMPRSS2 comprising residue Glu299. (xxxix) The present bispecific antibody specifically binds to an epitope on hTMPRSS2 comprising residue Asp345. (xl) The present bispecific antibody specifically binds to an epitope on hTMPRSS2 comprising residue Asn368. (xli) The present bispecific antibody specifically binds to an epitope on hTMPRSS2 comprising residue Pro369. (xlii) The present bispecific antibody specifically binds to an epitope on hTMPRSS2 comprising residue Gly370. (xliii) The present bispecific antibody specifically binds to an epitope on hTMPRSS2 comprising residue Met371. (xliv) The present bispecific antibody specifically binds to an epitope on hTMPRSS2 comprising residue Met372. (xlv) The present bispecific antibody specifically binds to an epitope on hTMPRSS2 comprising residue Leu373. (xlvi) The present bispecific antibody specifically binds to an epitope on hTMPRSS2 comprising residue Gln374. (xlvii) The present bispecific antibody specifically binds to an epitope on hTMPRSS2 comprising residue Glu376. (xlviii) The present bispecific antibody specifically binds to an epitope on hTMPRSS2 comprising residue Gln377. (xlix) The present bispecific antibody specifically binds to an epitope on hTMPRSS2 comprising residue Leu378. (l) The present bispecific antibody specifically binds to an epitope on hTMPRSS2 comprising residue Asp435. (li) The present bispecific antibody specifically binds to an epitope on hTMPRSS2 comprising residue Ser436. (lii) The present bispecific antibody specifically binds to an epitope on hTMPRSS2 comprising residue Gln438. (liii) The present bispecific antibody specifically binds to an epitope on hTMPRSS2 comprising residue Asp440. (liv) The present bispecific antibody specifically binds to an epitope on hTMPRSS2 comprising residue Ser441. (lv) The present bispecific antibody specifically binds to an epitope on hTMPRSS2 comprising residue Thr447. (lvi) The present bispecific antibody specifically binds to an epitope on hTMPRSS2 comprising residue Lys449. (lvii) The present bispecific antibody specifically binds to an epitope on hTMPRSS2 comprising residue Asn450. (lvii) The present bispecific antibody specifically binds to an epitope on hTMPRSS2 comprising residue Asn451. (lix) The present bispecific antibody specifically binds to an epitope on hTMPRSS2 comprising residue Ile452. (lx) The present bispecific antibody specifically binds to an epitope on hTMPRSS2 comprising residue Trp454. (lxi) The present bispecific antibody specifically binds to an epitope on hTMPRSS2 comprising residue Thr459. (lxii) The present bispecific antibody specifically binds to an epitope on hTMPRSS2 comprising residue Ser460. (lxiii) The present bispecific antibody specifically binds to an epitope on hTMPRSS2 comprising residue Trp461. (lxiv) The present bispecific antibody specifically binds to an epitope on hTMPRSS2 comprising residue Gly464. (lxv) The present bispecific antibody specifically binds to an epitope on hTMPRSS2 comprising residue Val473. (lxvi) The present bispecific antibody specifically binds to an epitope on hTMPRSS2 comprising residue Tyr474. (lxvii) The present bispecific antibody specifically binds to an epitope on hTMPRSS2 comprising residue Val65.

In a first preferred embodiment, the present bispecific antibody has a low effector function. In a second preferred embodiment, the present bispecific antibody has a long serum half-life. In a third preferred embodiment, the present bispecific antibody is an IgG4 antibody. In a fourth preferred embodiment, the present bispecific antibody comprises a heavy chain modification that inhibits half antibody formation. In a fifth preferred embodiment, the present bispecific antibody (i) has a low effector function; (ii) has a long serum half-life; (iii) is an IgG4 antibody; and (iv) comprises a heavy chain modification that inhibits half antibody formation.

In a preferred embodiment, the present bispecific antibody is a humanized bispecific antibody, and preferably a human bispecific antibody.

The following eight embodiments of the present bispecific antibody are exemplary. In a first embodiment of the invention, the present bispecific antibody is a humanized or human IgG4 antibody that (i) has the serum half-life-extending mutation combination M252Y/S254T/T256E (YTE) (with numbering according to the EU Index); (ii) has the half antibody formation-inhibiting mutation S228P or K447del, or the mutation combination S228P/K447del (with numbering according to the EU Index); and (iii) has the effector function-lowering L235E mutation (with numbering according to the EU Index).

In a second embodiment of the invention, the present bispecific antibody is a humanized or human IgG4 antibody that (i) has the serum half-life-extending mutation combination M252Y/S254T/T256E (YTE) (with numbering according to the EU Index); (ii) has the half antibody formation-inhibiting mutation S228P or K447del, or the mutation combination S228P/K447del (with numbering according to the EU Index); and (iii) has one or more of the effector function-lowering mutations L235A, F234A, and G237A (with numbering according to the EU Index).

In a third embodiment of the invention, the present bispecific antibody is a humanized or human IgG4 antibody that (i) has the serum half-life-extending mutation combination M252Y/S254T/T256E (YTE) (with numbering according to the EU Index); (ii) has the half antibody formation-inhibiting mutation S228P or K447del, or the mutation combination S228P/K447del (with numbering according to the EU Index); and (iii) has the effector function-lowering D265A mutation (with numbering according to the EU Index).

In a fourth embodiment of the invention, the present bispecific antibody is a humanized or human IgG4 antibody that (i) has the serum half-life-extending mutation combination M252Y/S254T/T256E (YTE) (with numbering according to the EU Index); (ii) has the half antibody formation-inhibiting mutation S228P or K447del, or the mutation combination S228P/K447del (with numbering according to the EU Index); and (iii) has one or more of the effector function-lowering mutations A330R, F243L, and an L328 substitution (with numbering according to the EU Index).

In a fifth embodiment of the invention, the present bispecific antibody is a humanized or human IgG4 antibody that (i) has the serum half-life-extending mutation combination M252Y/S254T/T256E (YTE) (with numbering according to the EU Index); (ii) has the half antibody formation-inhibiting mutation S228P or K447del, or the mutation combination S228P/K447del (with numbering according to the EU Index); and (iii) has the effector function-lowering IgG2/IgG4 format wherein IgG2 (up to T260) is joined to IgG4 (with numbering according to the EU Index).

In a sixth embodiment of the invention, the present bispecific antibody is a humanized or human IgG4 antibody that (i) has the serum half-life-extending mutation combination M252Y/S254T/T256E (YTE) (with numbering according to the EU Index); (ii) has the half antibody formation-inhibiting mutation S228P or K447del, or the mutation combination S228P/K447del (with numbering according to the EU Index); and (iii) has the effector function-lowering F243A/V264A mutation combination (with numbering according to the EU Index).

In a seventh embodiment of the invention, the present bispecific antibody is a humanized or human IgG4 antibody that (i) has the serum half-life-extending mutation combination M252Y/S254T/T256E (YTE) (with numbering according to the EU Index); (ii) has the half antibody formation-inhibiting mutation S228P or K447del, or the mutation combination S228P/K447del (with numbering according to the EU Index); and (iii) has the effector function-lowering E233P/F234A/L235A/G236del/G237A mutation combination (with numbering according to the EU Index).

In an eighth embodiment of the invention, the present bispecific antibody is a humanized or human IgG4 antibody that (i) has the serum half-life-extending mutation combination M252Y/S254T/T256E (YTE) (with numbering according to the EU Index); (ii) has the half antibody formation-inhibiting mutation S228P or K447del, or the mutation combination S228P/K447del (with numbering according to the EU Index); and (iii) has the effector function-lowering S228P/L235E mutation combination (with numbering according to the EU Index).

In a preferred embodiment of each of the above eight embodiments, the present bispecific antibody has a “knobs-into-holes” (kih) modification to prevent heavy chain mispairing. In another preferred embodiment of each of the above eight embodiments, the present bispecific antibody comprises two distinct heavy chains and two identical light chains. In a further preferred embodiment of each of the above eight embodiments wherein the antibody comprises two distinct heavy chains and two identical light chains, one of the heavy chains contains a chimeric Fc form that ablates binding to Protein A via the contact region. This technology, known as FcΔAdp, is described in M. Godar, et al., and A. D. Tustian, et al. The following additional two embodiments of the present bispecific antibody are exemplary. In a first embodiment of the invention, the present bispecific antibody is a humanized IgG4 antibody that (i) binds to the extracellular portion of hACE2 with an affinity of 50 nM; (ii) binds to the extracellular portion of hTMPRSS2 with an affinity of 50 nM; (iii) reduces the entry into hACE2⁺/hTMPRSS2⁺ human cells of a pseudovirus bearing SARS-CoV-2 S protein by a factor of 10,000; (iv) reduces the ability of human TMPRSS1 to cleave its substrate by 20%; (v) inhibits by 20% the ability of hACE2 to cleave a synthetic MCA-based peptide (preferably Mca-APK(Dnp); (vi) has the serum half-life-extending mutation combination M252Y/S254T/T256E (YTE) (with numbering according to the EU Index); (vii) has the half antibody formation-inhibiting mutation S228P or K447del, or the mutation combination S228P/K447del (with numbering according to the EU Index); and (viii) has an effector function-lowering mutation, mutation combination, or alteration, selected from the group consisting of L235E, L235A, F234A, G237A, D265A, A330R, F243L, L328 substitution, F243A/V264A, E233P/F234A/L235A/G236del/G237A, S228P/L235E, and an IgG2/IgG4 format wherein IgG2 (up to T260) is joined to IgG4 (with numbering according to the EU Index).

In a second embodiment of the invention, the present bispecific antibody is a human IgG4 antibody that (i) binds to the extracellular portion of hACE2 with an affinity of 50 nM; (ii) binds to the extracellular portion of hTMPRSS2 with an affinity of 50 nM; (iii) reduces the entry into hACE2⁺/hTMPRSS2⁺ human cells of a pseudovirus bearing SARS-CoV-2 S protein by a factor of 10,000; (iv) reduces the ability of human TMPRSS1 to cleave its substrate by 20%; (v) inhibits by 20% the ability of hACE2 to cleave a synthetic MCA-based peptide (preferably Mca-APK(Dnp); (vi) has the serum half-life-extending mutation combination M252Y/S254T/T256E (YTE) (with numbering according to the EU Index); (vii) has the half antibody formation-inhibiting mutation S228P or K447del, or the mutation combination S228P/K447del (with numbering according to the EU Index); and (viii) has an effector function-lowering mutation, mutation combination, or alteration, selected from the group consisting of L235E, L235A, F234A, G237A, D265A, A330R, F243L, L328 substitution, F243A/V264A, E233P/F234A/L235A/G236del/G237A, S228P/L235E, and an IgG2/IgG4 format wherein IgG2 (up to T260) is joined to IgG4 (with numbering according to the EU Index).

In a preferred embodiment of each of the above two embodiments, the present bispecific antibody has a “knobs-into-holes” (kih) modification to prevent heavy chain mispairing. In another preferred embodiment of each of the above two embodiments, the present bispecific antibody comprises two distinct heavy chains and two identical light chains. In a further preferred embodiment of each of the above two embodiments wherein the antibody comprises two distinct heavy chains and two identical light chains, one of the heavy chains contains a chimeric Fc form that ablates binding to Protein A via the contact region (i.e., FcΔAdp technology).

This invention provides an isolated nucleic acid molecule encoding (a) the present bispecific antibody, if the bispecific antibody has only one chain; or (b) one or more chains of the present bispecific antibody, if the bispecific antibody has a plurality of chains. Where the present bispecific antibody comprises light and heavy chains, this invention also provides an isolated nucleic acid molecule encoding (i) the complete light chain, or a portion of the light chain, of the present bispecific antibody, and/or (ii) the complete heavy chain, or a portion of the heavy chain, of the present bispecific antibody. In one embodiment, the present nucleic acid molecule is a DNA molecule, for example, a cDNA molecule.

This invention further provides a recombinant vector, for example a plasmid or a viral vector, comprising the present nucleic acid molecule operably linked to a promoter of RNA transcription.

This invention still further provides a host vector system comprising one or more of the present vectors in a suitable host cell (e.g., a bacterial cell, an insect cell, a yeast cell, or a mammalian cell such as a hybridoma cell (See, e.g., Chiu and Gilliland; Kohler and Milstein)).

This invention further provides a composition comprising (i) the present bispecific antibody, and (ii) a pharmaceutically acceptable carrier.

This invention provides a method for reducing the likelihood of a human subject's becoming infected with SARS-CoV-2 comprising administering to the subject a prophylactically effective amount of the present bispecific antibody. In a preferred embodiment of this method, the subject has been exposed to SARS-CoV-2. In another preferred embodiment of this method, the present bispecific antibody does not exhibit significant toxicity in a cynomolgus monkey when administered at a prophylactically effective amount. As an example, when administered at a prophylactically effective amount to a cynomolgus monkey, the present bispecific antibody does not cause more than a 15% fluctuation in blood pressure or in the number of white blood cells, red blood cells, monocytes, or lymphocytes. Methods for determining toxicity in cynomolgus monkeys are presented in the examples below.

This invention also provides a method for treating a human subject who is infected with SARS-CoV-2 comprising administering to the subject a therapeutically effective amount of the present bispecific antibody. In one embodiment of this method, the subject is symptomatic of a SARS-CoV-2 infection. In another embodiment, the subject is asymptomatic of a SARS-CoV-2 infection. In another preferred embodiment of this method, the present bispecific antibody does not exhibit significant toxicity in a cynomolgus monkey when administered at a therapeutically effective amount. As an example, when administered at a therapeutically effective amount to a cynomolgus monkey, the present bispecific antibody does not cause more than a 15% fluctuation in blood pressure or in the number of white blood cells, red blood cells, monocytes, or lymphocytes.

This invention provides a recombinant AAV vector comprising a nucleic acid sequence encoding (a) the present bispecific antibody, if the bispecific antibody has only one chain, or (b) one or more chains of the present bispecific antibody, if the bispecific antibody has a plurality of chains. In one embodiment of the present recombinant AAV vector, the nucleic acid sequence encodes all chains of the bispecific antibody. In another embodiment, the nucleic acid sequence encodes one or more chains of the bispecific antibody, but not all chains.

In connection with the present vectors, a nucleic acid sequence “encoding” a protein (e.g., an antibody heavy chain) encodes it operably (i.e., in a manner permitting its expression in a cell infected by a viral particle comprising the vector that contains the nucleic acid sequence). Additionally, the recombinant viral vectors of this invention are not limited to any particular configuration with respect to the exogenous protein-coding sequences. For example, in one embodiment of the subject recombinant AAV vector, a “one vector” approach is used wherein a singular recombinant AAV vector includes nucleic acid sequences encoding an scFv bispecific antibody. In another embodiment, a “two vector” approach is used wherein one recombinant AAV vector includes a nucleic acid sequence encoding a first heavy antibody chain and a first light antibody chain, and a second recombinant AAV vector includes a nucleic acid sequence encoding a second heavy antibody chain and a second light antibody chain (See, e.g., S. P. Fuchs, et al. (2016)).

This invention further provides a recombinant AAV particle comprising the present recombinant AAV vector and an AAV capsid protein.

This invention also provides a composition comprising (i) a plurality of the present AAV particles and (ii) a pharmaceutically acceptable carrier.

This invention provides a method for reducing the likelihood of a human subject's becoming infected with SARS-CoV-2 comprising administering to the subject a prophylactically effective number of the present AAV particles. In one embodiment of the present prophylactic method, the subject has been exposed to SARS-CoV-2. In another embodiment, the subject has not been exposed to SARS-CoV-2.

This invention provides a method for treating a human subject who is infected with SARS-CoV-2 comprising administering to the subject a therapeutically effective number of the present AAV particles. In one embodiment of the present therapeutic method, the subject is symptomatic of a SARS-CoV-2 infection. In another embodiment, the subject is asymptomatic of a SARS-CoV-2 infection.

This invention further provides a kit comprising, in separate compartments, (a) a diluent and (b) the present bispecific antibody either as a suspension or in lyophilized form.

Finally, this invention provides a kit comprising, in separate compartments, (a) a diluent and (b) a suspension of a plurality of the present recombinant AAV particles. In one example, the subject kit comprises (i) a single-dose vial containing a concentrated solution of the subject particles (also measured as viral genomes) in a suitable solution (e.g., a solution of sterile water, sodium chloride, sodium phosphate, and Poloxamer 188) and (ii) one or more vials of suitable diluent (e.g., a solution of sterile water, sodium chloride, sodium phosphate, and Poloxamer 188).

The present vectors, particles, and methods are envisioned for suitable recombinant non-AVV viruses (e.g., lentivirus, adenovirus, alphavirus, herpesvirus, or vaccinia virus), mutatis mutandis, as they are for recombinant AAV viruses in this invention.

The present antibodies, vectors, particles, and methods are envisioned for all viruses (e.g., SARS-CoV, MERS-CoV, and influenza viruses (e.g., H1N1, H2N2, H3N2, H5N1, H1N2, and H7N9) that depend on proteolytic cleavage by TMPRSS2 for cellular entry, mutatis mutandis, as they are for SARS-CoV-2 in this invention.

The present bispecific antibodies, compositions, vectors, particles, and methods are envisioned for the prophylaxis and treatment of all SARS-CoV viruses other than SARS-CoV-2 (e.g., SARS-CoV), mutatis mutandis, as they are for the prophylaxis and treatment of SARS-CoV-2 in this invention.

This invention will be better understood by reference to the examples which follow, but those skilled in the art will readily appreciate that the specific examples detailed are only illustrative of the invention as described more fully in the claims that follow thereafter.

### EXAMPLES

### Example 1—BioVision Assay Kit for ACE2 Function

BioVision, Inc. sells the Angiotensin II Converting Enzyme (ACE2) Activity Assay Kit (Fluorometric) (https://www.biovision.com/angiotensin-ii-converting-enzyme-ace2-activity-assay-kit-fluorometric.html). This kit can be used to measure the degree to which an antibody inhibits the ability of hACE2 to cleave angiotensin II.

BioVision provides the following background information regarding its test kit, which has been edited here. Angiotensin II converting enzyme (ACE2), a zinc-based metalloprotease, is part of the renin-angiotensin system (RAS) that controls the regulation of blood pressure by cleaving the C-terminal amino acid residue of Angiotensin II to convert it into Angiotensin 1-7. ACE2 is a receptor of human coronaviruses, such as SARS and HCoV-NL63. It is expressed on the vascular endothelial cells of lung, kidney, and heart. ACE2 is a potential therapeutic target for cardiovascular and coronavirus-induced diseases. BioVision's kit will aid research in this field. It utilizes the ability of an active ACE2 to cleave a synthetic MCA-based peptide substrate to release a free fluorophore. The released MCA can be easily quantified using a fluorescence microplate reader. BioVision also provides an ACE2-specific inhibitor that can differentiate the ACE2 activity from other proteolytic activity. This kit can detect as low as 0.4 mU, is simple, and can be used in a high-throughput format.

BioVision's kit has the following specifications: (i) Cat #—K897-100; (ii) Size—100 assays; (iii) Detection Method—Fluorometric (Ex/Em=320/420 nm); (iv) Species Reactivity—Mammalian; (v) Applications—Detection of ACE2 activity in tissue/cell lysates and enzyme preparations; (vi) Features & Benefits—Simple one-step reaction/Takes only 1-2 hrs/Non-radiometric fluorescent detection/HTP adaptable; (vii) Kit Components—ACE2 Assay Buffer/ACE2 Dilution Buffer, and ACE2 Lysis Buffer/ACE2 Positive Control, ACE2 Substrate, ACE2 Inhibitor (22 mM), and MCA-Standard (1 mM); (viii) Storage Conditions—−20° C.; and (ix) Shipping Conditions—Gel Pack.

### Example 2—SensoLyte Assay Kit for ACE2 Function

Anaspec sells the SensoLyte® 390 ACE2 Activity Assay Kit *Fluorimetric* (“SensoLyte kit”) (https://www.anaspec.com/products/product.asp?id=43987). This kit can be used to measure the degree to which an antibody inhibits the ability of hACE2 to cleave angiotensin II.

Anaspec provides the following information regarding its SensoLyte test kit, which has been edited here. The kit provides a convenient assay for high throughput screening of ACE2 enzyme inhibitors and inducers using a Mc-Ala/Dnp fluorescence resonance energy transfer (FRET) peptide. In the FRET peptide, Dnp quenches the fluorescence of Mc-Ala. Upon cleavage into two separate fragments by ACE2, the fluorescence of Mc-Ala is recovered, and can be monitored at excitation/emission=330/390 nm. This assay can detect the activity of sub-nanogram levels of ACE2. Assays are performed in a convenient 96-well microplate format.

The Sensolyte kit also has the following specifications: (i) Cat #—AS-72086; (ii) Size—100 assays; (iii) Storage Conditions—−20° C.

### Example 3—Angiotensin II-Based Mass Spectrometry Assay for hACE2 Function

This method (the “mass spectrometry assay”) can be used to quantitatively measure hACE2 activity using mass spectrometry. In particular, it can be used to measure the degree to which an antibody inhibits the ability of hACE2 to cleave angiotensin II, as well as other substrates. The method is adapted from the ACE2 assay described in Donoghue, et al.

Enzymatic reactions are performed in 15 μl. To each tube at room temperature is added 10 μl of buffer (10 mmol/l Tris, pH 7.0) with or without hACE2. The hACE2 used in this method is recombinant soluble hACE2 prepared according to Donoghue, et al. Five microliters of purified angiotensin II (Sigma) are added to each tube for a final concentration of 5 μmol/I. (This mass spectrometry assay can also employ peptide substrates other than angiotensin II (e.g., des-Arg-bradykinin, neurotensin, kinetensin, apelin-13, and dynorphin A 1-13).) Lisinopril or captopril (Sigma) is added to some reactions at final concentrations of 6.6 μmol/I. Neither lisinopril nor captopril inhibits hACE2 activity, and these compounds are thus useful as controls to ensure that the angiotensin II cleavage measured is due to hACE2 activity. For reactions and control experiments, the tubes are incubated at 37° C. for 30 minutes. A portion (1 μl) of each reaction is quenched by the addition of 1 μl of a low-pH MALDI matrix compound (10 g/L α-cyano-4 hydroxycinnamic acid in a 1:1 mixture of acetonitrile and water). One microliter of the resulting solution is applied to the surface of a MALDI plate. The plate is then air-dried and inserted into the sample introduction port of the Voyager Elite biospectrometry MALDI time-of-flight (TOF) mass spectrometer (PerSeptive Biosystems). The resulting signal is digitized at a frequency of 1 GHz and accumulated for 64 scans. Purified conditioned medium from empty vector transfections is used to control individual experiments for variability in extent of substrate conversion to product. For tandem mass spectrometry sequencing, a hybrid quadrupole time-of-flight mass spectrometer (Q-TOF-MS) (Micromass UK Limited) equipped with an orthogonal electrospray source (Z-spray) is used. The quadrupole is set up to pass precursor ions of selected m/z to the hexapole collision cell (Q2), and product ion spectra are acquired with the TOF analyzer. Argon is introduced into the Q2 with a collision energy of 35 eV and cone energy of 25 V.

### Example 4—Angiotensin II-Based HPLC Assay for hACE2 Function

This method (the “HPLC assay”) can be used to quantitatively measure hACE2 activity using HPLC. In particular, it can be used to measure the degree to which an antibody inhibits the ability of hACE2 to cleave angiotensin II, as well as other substrates. The method is adapted from the “ACEH” assay described in Tipnis, et al.

### Protein and Enzymatic Assays.

Protein concentrations are determined using the bicinchoninic acid assay (Smith, et al.) with bovine serum albumin as a standard. Assays for hACE2 activity are carried out in a total volume of 100 μl, containing 100 mM Tris-HCl, pH 7.4, 20 μg of protein and 100 μM angiotensin II as a substrate. (This HPLC assay can also employ peptide substrates other than angiotensin II (e.g., des-Arg-bradykinin, neurotensin, and kinetensin, apelin-13, and dynorphin A 1-13).) Where appropriate, inhibitors are added to give final concentrations of 10 μM lisinopril, 10 μM captopril, 10 μM enalaprilat, 100 μM benzyl succinate, or 10 mM EDTA. EDTA inhibits hACE2 activity, but none of lisinopril, captopril, enalaprilat, and benzyl succinate (a carboxypeptidase A inhibitor) inhibits hACE2 activity. These compounds are thus useful as controls to ensure that the angiotensin II cleavage measured is due to hACE2 activity. Reactions are carried out at 37° C., for 2 hours and stopped by heating to 100° C. for 5 minutes followed by centrifugation at 11,600×g for 10 minutes. Carboxypeptidase A assays are carried out at room temperature for 30 minutes, using 0.1 units of enzyme per assay.

### HPLC Analysis of Cleavage Products.

Peptide hydrolysis products are separated using reverse-phase HPLC (pBondapak C-18 reverse phase column, Waters) with a UV detector set at 214 nm. All separations are carried out at room temperature, with a flow rate of 1.5 ml/min. Mobile phase A consists of 0.08% (v/v) phosphoric acid and mobile phase B consists of 40% (v/v) acetonitrile in 0.08% (v/v) phosphoric acid. A linear solvent gradient of 11% B to 100% B over 15 minutes with five minutes at final conditions, and eight minute re-equilibration is used. The product from angiotensin II is collected and analyzed by matrix-assisted laser desorption ionization/time-of-flight mass spectrometry.

### Example 5—Protease Assays

The assays in Examples 5-7, adapted from Koschubs, et al., are described for hepsin (i.e., TMPRSS1). However, they can also be performed on other proteases such as recombinant HAT (i.e., TMPRSS11D) and human matriptase.

Purified hepsin is diluted to 1 nM in assay buffer [50 mM Tris/HCl (pH 7.4), 100 mM NaCl, 0.1 mg/ml BSA and 0.02% Tween 20]. Acetyl-KQLR-AMC peptide (AMC is 7-amino-4-methylcoumarin) is synthesized with >95% purity as determined by HPLC and MS analysis.

For measuring amidolytic activities, hepsin is transferred to a 384-well flat-bottomed plate (Optiplate, PerkinElmer). The acetyl-KQLR-AMC peptide (5 μM) is added and the enzyme reaction is started. Assays contain less than 5% DMSO in a final test volume of 30 μl. The fluorescence increase is monitored with excitation at 530 nm and emission at 572 nm on an Envision reader (PerkinElmer) at 26° C. To determine the apparent Kₘ value and inhibition model, hydrolysis rates of at least six different concentrations of peptide are measured in triplicate. Rates of hydrolysis and apparent Kₘ values are calculated using XLFit software (IDBS).

Progress curves of the steady-state reactions are analyzed by adding 0.5 nM hepsin to a mixture of 10 μM acetyl-KQLR-AMC peptide and 18-500 nM antibody. Fluorescence is measured on a Carey Eclipse Fluorescence Spectrophotometer for two minutes at 26° C. Monitoring of the enzyme reaction starts after a delay of approximately two seconds. Rates for initial and steady state reactions are calculated using linear regression analysis XLFit software (IDBS).

To evaluate the inhibition mechanism, various concentrations of antibody (20-0.31 nM in two-fold dilutions in triplicate) are incubated with 1 nM hepsin for 15 minutes. The linear rates of fluorescence increase are measured after simultaneously adding 20, 10, 5, and 2.5 μM acetyl-KQLR-AMC peptide. Data are fitted to the equations for tight binding inhibition using SigmaPlot enzyme kinetic software (Version 8.02, Systat).

### Example 6—Protease Inhibition by Antibodies

To determine inhibitory activities, hepsin (1 nM) and dilutions of antibodies are transferred to a 384-well flat-bottomed plate (Optiplate, PerkinElmer) and incubated for 30 minutes at 26° C. Peptide (5 μM) is added and the enzyme reaction is started. After 40 minutes of incubation at 26° C., the fluorescence increase is measured with excitation at 530 nm and emission at 572 nm on an Envision reader (PerkinElmer).

The percentage inhibition of hepsin activity is calculated according to the following formula:

% Inhibition=100×[1−(Fₛ−F_{b})/(Fₜ−F_{b})]

where Fₛ is the fluorescence signal of the sample including the antibody, F_{b} is the fluorescence signal in the absence of hepsin and antibody, and Fₜ is the fluorescence signal in the presence of hepsin with no antibody. The concentration of inhibitor resulting in 50% inhibition (IC₅₀) of the uninhibited enzyme is calculated after fitting the data to a four-parameter equation using XLFit® software (IDBS). At least three independent measurements are performed in triplicate.

### Example 7—FRET Activity Assay

Antibody specificity is tested using a FRET (fluorescence resonance energy transfer) activity assay with JA133-Z-Gln-Arg-Arg-Z-Lys-(TAMRA™)-NH₂ (synthesized and purified as described in Koschubs, et al.) as the cleavable peptide. Purified human hepsin is diluted in assay buffer (see above) to a concentration of 10 nM. Peptide substrate is diluted in assay buffer to 300 nM and antibody to 0.293 nM. Then, 10 μl of diluted hepsin and antibody solutions are each added into 384-well microtitre plates and incubated at room temperature (20° C.) for 30 minutes. Peptide substrate (10 μl/well) is added to each well, mixed, and incubated at room temperature for 60 minutes. Signals are quantified by reading fluorescence (excitation at 530 nm and emission at 572 nm) on a Victor 2 reader (PerkinElmer). The percent inhibition of hepsin activity is calculated as described above.

### Example 8—Hepsin (TMPRSS1) Activity Assay

This assay, adapted from Chevillet, et al., is described for hepsin (i.e., TMPRSS1). However, it can also be performed on other proteases such as trypsin and thrombin.

Titration of the chromogenic substrate pyroGlu-Pro-Arg-pNA is performed for hepsin and the resulting substrate-velocity data are fitted with non-linear regression using GraphPad Prism 4 to calculate Vₘₐₓ and Kₘ. Enzyme assay concentration and Kₘ for hepsin are 0.4 nM and 170 μM, respectively. Inhibitor (i.e., antibody) activity is determined by incubating hepsin with increasing concentrations of inhibitor for 30 minutes at room temperature followed by addition of the substrate at the appropriate Kₘ. The reactions are then followed using a kinetic microplate reader and the linear rates of increase in absorbance at 405 nm expressed as residual percent activity (100%×vᵢ/vₒ). At least three independent experiments are performed for hepsin. IC₅₀ is calculated by fitting the data to a four-parameter nonlinear regression using GraphPad Prism 4. The equilibration time-dependence of inhibitor potency is determined by incubating hepsin with the respective inhibitor at its IC₅₀ value or buffer/solvent alone under the above conditions in triplicate. Samples are withdrawn at 30, 60, 120, and 180 minutes and activity analyzed by the addition of substrate as above. The reversibility of inhibition is determined using a dilution technique. Hepsin is incubated with the inhibitors at their respective IC₅₀ values or buffer control as above for one hour at room temperature in triplicate. Samples are then diluted with buffer to the additional percentage indicated, and activity is measured as above.

### Example 9—Measuring Interaction of Soluble RBD Protein with Soluble hACE2

In a preferred embodiment of this invention, measuring the interaction of soluble RBD protein (a proxy for SARS-CoV-2) with soluble hACE2 (a proxy for the extracellular portion of hACE2) can be used to indirectly measure (i) the binding of a monoclonal antibody to the extracellular portion of hACE2, and (ii) a monoclonal antibody's ability to inhibit binding of SARS-CoV-2 to the extracellular portion of hACE2.

The following method for analyzing hACE2-binding inhibition is taken from Suryadevara, et al. Wells of 384-well microtiter plates are coated with 1 μg/mL purified recombinant SARS-CoV-2 S2Pecto protein at 4° C. overnight. Plates are blocked with 2% non-fat dry milk and 2% normal goat serum in DPBS-T for 1 hour. For screening assays, purified monoclonal antibodies are diluted two-fold in blocking buffer starting from 10 μg/mL in triplicate, added to the wells (20 μL per well) and incubated for 1 hour at ambient temperature. Recombinant hACE2 with a C-terminal Flag tag peptide is added to wells at 2 μg/mL in a 5 μL per well volume (final 0.4 μg/mL concentration of hACE2) without washing of antibody and then incubated for 40 minutes at ambient temperature. Plates are washed and bound hACE2 is detected using HRP-conjugated anti-Flag antibody (Sigma-Aldrich, cat. A8592, lot SLBV3799, 1:5,000 dilution) and TMB substrate. ACE2 binding without antibody serves as a control. The signal obtained for binding of the human ACE2 in the presence of each dilution of tested antibody is expressed as a percentage of the human ACE2 binding without antibody after subtracting the background signal. For dose-response assays, serial dilutions of purified monoclonal antibodies are applied to the wells in triplicate, and monoclonal antibody binding is detected as detailed above. IC50 values for inhibition by monoclonal antibody of S2Pecto protein binding to human ACE2 are determined after log transformation of antibody concentration using sigmoidal dose-response nonlinear regression analysis.

The reagents used in this example can be made according to this reference and/or purchased commercially (e.g., from LakePharma, Inc., Worcester, Mass.). In addition, related kits are commercially available. For example, (i) a SARS-CoV-2 Spike-ACE2 Interaction Inhibitor Screening Assay Kit is available from Cayman Chemical (Ann Arbor, Mich.); and (ii) a SARS-CoV-2 Spike:ACE2 Inhibitor Screening Assay Kit, an ACE2 Inhibitor Screening Assay Kit, and a Spike RBD (SARS-CoV-2): ACE2 Inhibitor Screening Assay Kit are all available from BPS Bioscience (San Diego, Calif.).

### Example 10—Recombinant hTMPRSS2 Assay

This enzymatic assay can be used to quantitatively measure the binding of an agent (e.g., an antibody) to recombinant hTMPRSS2. In particular, it can be used to measure the degree to which an antibody specifically binds to the extracellular portion of human hTMPRSS2. The assay is exemplified using TMPRSS2-binding small molecules (i.e., camostat, nafamostat, and gabexate). The method is adapted from the hTMPRSS2 assay described in Shrimp, et al.

Reagents

Recombinant human TMPRSS2 protein expressed from yeast (human TMPRSS2 residues 106-492, N-terminal 6×His-tag) (cat. #TMPRSS2-1856H) is acquired from Creative BioMart (Shirley, N.Y.). Peptides obtained from Bachem include Boc-Leu-Gly-Arg-AMC. Acetate (cat. #I-1105), Boc-Gln-Ala-Arg-AMC. HCl (cat. #I-1550), Ac-Val-Arg-Pro-Arg-AMC. TFA (cat. #I-1965), Cbz-Gly-Gly-Arg-AMC. HCl (cat. #I-1140). Peptides custom ordered from LifeTein (Somerset, N.J.) include Cbz-d-Arg-Gly-Arg-AMC, and Cbz-d-Arg-Pro-Arg-AMC.

Fluorogenic Peptide Screening Protocol-384-Well Plate

To a 384-well black plate (Greiner 781900) is added Boc-Gln-Ala-Arg-AMC (62.5 nL) and inhibitor (62.5 nL) using an ECHO 655 acoustic dispenser (LabCyte). To that is added TMPRSS2 (750 nL) in assay buffer (50 mM Tris pH 8, 150 mM NaCl, 0.01% Tween20) to give a total reaction volume of 25 μL. Following 1 hour incubation at RT, detection is done using the PHERAstar with 340 nm excitation and 440 nm emission.

Fluorescence Counter Assay-384-Well Plate

To a 384-well black plate (Greiner 781900) is added 7-amino-methylcoumarin (62.5 nL) and inhibitor or DMSO (62.5 nL) using an ECHO 655 acoustic dispenser (LabCyte). To that is added assay buffer (50 mM Tris pH 8, 150 mM NaCl, 0.01% Tween20) to give a total reaction volume of 25 μL. Detection is done using the PHERAstar with 340 nm excitation and 440 nm emission. Fluorescence is normalized relative to a negative control containing DMSO-only wells (0% activity, low fluorescence) and a positive control containing AMC only (100% activity, high fluorescence). An inhibitor causing fluorescence quenching would be identified as having a concentration-dependent decrease on AMC fluorescence.

Fluorogenic Peptide Screening Protocol-1536-Well Plate

To a 1536-well black plate is added Boc-Gln-Ala-Arg-AMC substrate (20 nL) and inhibitor (20 nL) using an ECHO 655 acoustic dispenser (LabCyte). To that is dispensed TMPRSS2 (150 nL) in assay buffer (50 mM Tris pH 8, 150 mM NaCl, 0.01% Tween20) using a BioRAPTR (Beckman Coulter) to give a total reaction volume of 5 μL. Following 1 hour of incubation at RT, detection is done using the PHERAstar with 340 nm excitation and 440 nm emission.

TMPRSS2 Assay Protocol

The TMPRSS2 biochemical assay is performed according to the assay protocol shown in the table below.

| Step | Process | Notes |
|---|---|---|
| 1 | 20 nL of peptide | Peptide (dissolved in DMSO) |
| | substrate dispensed | dispensing performed using an |
| | into 1536-well plates. | ECHO 655 acoustic dispenser |
| | | (LabCyte). Corning 1536-well |
| | | Black Polystyrene, square well, |
| | | high base, nonsterile, nontreated; |
| | | cat.# 3724 |
| 2 | 20 nL of inhibitor | Inhibitor or vehicle control |
| | or vehicle control | (DMSO) dispensing performed |
| | (DMSO) dispensed | using an ECHO 655 acoustic |
| | into 1536-well plates. | dispenser (LabCyte). |
| 3 | TMPRSS2 diluted | TMPRSS2 (33.5 μM, 150 nL) |
| | in assay buffer | in assay buffer (50 mM Tris |
| | dispensed into | pH 8, 150 mM NaCl, 0.01% |
| | 1536-well plates. | Tween20) dispensing performed |
| | | using a BioRAPTR (Beckman |
| | | Coulter). Total reaction volume |
| | | of 5 μL. |
| 4 | Incubate at RT | Final assay conditions are 10 μM |
| | for 1 h | peptide and 1 μM TMPRSS2 in |
| | | assay buffer (50 mM Tris-HCl, |
| | | pH 8, 150 mM NaCl, |
| | | 0.01% Tween20) |
| 5 | Read on PHERAstar | Fastest read settings, Fluorescence |
| | FSX (BMG Labtech) | Intensity module, 340 nm excitation, |
| | | 440 nm emission) (cat.# 1601A2, |
| | | BMG Labtech) |

Data Process and Analysis

To determine compound activity in the assay, the concentration-response data for each sample are plotted and modeled by a four-parameter logistic fit yielding IC₅₀ and efficacy (maximal response) values. Raw plate reads for each titration point are first normalized relative to a positive control containing no enzyme (0% activity, full inhibition) and a negative control containing DMSO-only wells (100% activity, basal activity). Data normalization, visualization, and curve fitting are performed using Prism (GraphPad, San Diego, Calif.).

Protease Profiling

Camostat, nafamostat, and gabexate are assessed for inhibition against panels of recombinant human proteases by commercial services from Reaction Biology Corp and BPS Biosciences. The Reaction Biology Corp profile tested in a 10-dose IC₅₀ with a 3-fold serial dilution starting at 10 μM against 65 proteases. The BPS Biosciences profile is against 48 proteases at a single concentration of 10 μM.

### Example 11—Production and Titration of Pseudoviruses

In one embodiment of this invention, pseudoviruses are produced and titrated according to the following method taken from Nie, et al.

For pseudovirus construction, spike genes from strain Wuhan-Hu-1 (GenBank: MN908947) are codon-optimized for human cells and cloned into eukaryotic expression plasmid pcDNA3.1 to generate the envelope recombinant plasmid pcDNA3.1.S2.

The pseudoviruses are produced and titrated using methods similar to Rift valley fever pseudovirus, as described previously (e.g., by Ma, et al., and Whitt). For this VSV pseudovirus system, the backbone is provided by VSV G pseudotyped virus (G*ΔG-VSV) that packages expression cassettes for firefly luciferase instead of VSV-G in the VSV genome. Briefly, 293T cells are transfected with pcDNA3.1.S2 (30 μg for a T75 flask) using Lipofectamine 3000 (Invitrogen, L3000015) following the manufacturer's instructions. Twenty-four hours later, the transfected cells are infected with G*ΔG-VSV with a multiplicity of four. Two hours after infection, cells are washed with PBS three times, and then new complete culture medium is added. Twenty-four hours post infection, SARS-CoV-2 pseudoviruses containing culture supernatants are harvested, filtered (0.45-μm pore size, Millipore, SLHP033RB) and stored at −70° C. in 2-ml aliquots until use. The 50% tissue culture infectious dose (TCID50) of SARS-CoV-2 pseudovirus is determined using a single-use aliquot from the pseudovirus bank. All stocks are used only once to avoid inconsistencies that could result from repeated freezing-thawing cycles. For titration of the SARS-CoV-2 pseudovirus, a 2-fold initial dilution is made in hexaplicate wells of 96-well culture plates followed by serial 3-fold dilutions (nine dilutions in total). The last column serves as the cell control without the addition of pseudovirus. Then, the 96-well plates are seeded with trypsin-treated mammalian cells adjusted to a pre-defined concentration. After 24 h incubation in a 5% CO2 environment at 37° C., the culture supernatant is aspirated gently to leave 100 μl in each well. Then, 100 μl of luciferase substrate (Perkinelmer, 6066769) is added to each well. Two minutes after incubation at room temperature, 150 μl of lysate is transferred to white solid 96-well plates for the detection of luminescence using a microplate luminometer (PerkinElmer, Ensight). The positive well is determined as ten-fold relative luminescence unit (RLU) values higher than the cell background. The 50% tissue culture infectious dose (TCID50) is calculated using the Reed-Muench method, as described previously.

### Example 12—Supplemental Antibody Generation and Testing Methods

In a preferred embodiment of this invention, the present antibody's hACE2-binding ability, hACE2 carboxypeptidase-inhibiting ability, virus-neutralizing ability, and toxicity can be determined using the following methods taken from Du, et al.

Cell Lines and Viruses

HEK293T (ATCC, CRL-3216), HEK293T-ACE2 (SinoBiological, OEC001), Vero E6 (ATCC, CRL-1586), and LLC-MK2 (ATCC, CRL-7) cells are cultured at 37° C. under 5% CO₂ in Dulbecco's modified Eagle's medium (DMEM) (HyClone, South Logan, Utah) supplemented with 10% fetal bovine serum (FBS) (Gibco, Carlsbad, Calif., USA).

SARS-CoV-2 virus (BetaCoV/Wuhan/IVDC-HB-01/2020, GISAID accession ID: EPI_ISL_402119) is used. Vero E6 cells are applied to the reproduction of SARS-CoV-2 stocks. The HCoV-NL63 strain is used. LLC-MK2 cells are applied to the reproduction of HCoV-NL63 stocks.

Generation of ACE2-Blocking Monoclonal Antibodies

To generate murine anti-hACE2 antibodies, BALB/c mice receive hACE2 (19-615) soluble antigens in a prime-boost immunization regimen with a 4-week interval. Using hybridoma technology, one obtainer a number of mouse anti-hACE2 cell clones. After screening hybridoma supernatants, several clones of the monoclonal antibodies that block HEK293T-hACE2 cell infection with SARS-CoV and SARS-CoV-2 spike pseudotyped virus are identified. The antibody clone exhibiting the best inhibitory activity against pseudotyped virus infection (top antibody) is identified. The sequences of the variable regions of the top antibody are obtained through rapid amplification of complementary DNA (cDNA) ends amplification.

Plasmid Construction

The coding sequences of SARS-CoV-RBD (residues 306-527, accession number: NC_004718), SARS-CoV-2-RBD (residues 319-541, accession number EPI_ISL_402119), hACE2 (residues 19-615, accession number BAJ21180), and hACE2 variants (S19P, I21T, K26R, N33D, and D38E) fused with N-terminal native signal peptides and C-terminal 6×His tag are, respectively, cloned into the pCAGGS expression vector (Addgene) using the EcoRI and XhoI restriction sites. The signal peptides and variable regions of antibody are synthesized (GenScript) and fused with the coding sequences for the human IgG4 and kappa light chain constant region into the pCAGGS vectors. The pEGFP-N1-hACE2 plasmid is constructed by cloning the coding region of hACE2 into pEGFP-N1 using restriction enzymes XhoI and SmaI. To express minimal glycosylated ACE2, a coding sequence of residues 19-615 is synthesized (GenScript) and cloned into pFastBac1 vector (Invitrogen), with an N-terminal gp67 signal peptide and a C-terminal 6×His tag.

Protein Expression and Purification

To prepare the proteins of ACE2 (19-615), SARS-CoV-RBD, and SARS-CoV-2-RBD, HEK293T cells are transiently transfected with expressing plasmids containing the coding sequence for the indicated proteins. After 3 days, the supernatant is collected and soluble protein is purified by Ni affinity chromatography using a HisTrap HP 5 ml column (GE Healthcare), The samples are then further purified via size-exclusion chromatography with a Superdex 200 column (GE Healthcare) in a buffer composed of 20 mM Tris-HCl (pH 8.0) and 150 mM NaCl. Preparation of the full-length protein is achieved by transfection of plasmids into HEK293T cells. The protein is purified from the culture supernatants using a HiTrap Protein A HP column (GE Healthcare) and subsequently purified via the above size-exclusion chromatography.

For crystal screenings, the peptidase domain of human ACE2 (19-615) with a C-terminal 6×His tag is expressed using the baculovirus-insect cell system. The baculovirus is generated and amplified using the Sf21 insect cells (Invitrogen, B82101), and Hi5 insect cells (Invitrogen, 885502) are used for protein expression. The conditioned medium is collected 48 h post infection and exchanged into the binding buffer (10 mM HEPES, pH 7.2, and 150 mM NaCl). The ACE2 (19-615) and antibody-Fab proteins are purified as described above for HEK293T cell-derived ACE2 (19-615), To obtain the complex between ACE2 and antibody-Fab, purified ACE2 and antibody-Fab are incubated together, passed through a Superdex 200 increase 10/300 gel filtration column (GE Healthcare), and eluted using the binding buffer.

Flow Cytometry Assay

To test the activity of antibodies to block the binding between ACE2 and SARS-CoV-RBD, or SARS-CoV-2-RBD. HEK293T cells are transiently transfected with pEGFP-N1-ACE2 plasmids. After 24 h, 3×10⁵ cells are collected and incubated with 10 μg/ml antibody protein or isotype IgG at 37° C. for 30 min, followed by incubation with 200 ng/ml RBD proteins at 37° C. for another 30 min, After washing three times, the cells are incubated with APC-conjugated anti-His antibody (1:200, Miltenyi Biotec, 130-119-782) for another 30 min. Then, the cells and data are collected and analyzed using flow cytometry (BD FACS Canto™ H, BD FACSDiva Software v8.0.3, and Flow Jo 7.6.1).

To test whether the antibody has any impact on the cell-surface expression of hACE2, HEK293T-hACE2 cells are incubated with different concentrations (10 μg/ml or with five-fold serial dilutions ranging from 10 μg/ml to 0.64 ng/ml) of antibody at 37° C. in DMEM with 10% FBS for 4 or 24 h. Then, the cells are washed with FACS buffer (phosphate-buffered saline (PBS), 1% bovine serum albumin, and 2 mM EDTA) and incubated with 10 μg/ml antibody or isotype IgG at 4° C. for 60 min. After washing three times, cells are incubated with Alexa Fluor™488 goat anti-human IgG (H+L) antibody (1:200, Invitrogen, A11013) at 4° C. for another 30 min. Then, the cells are washed twice and resuspended in 200 μl FAGS buffer for flow cytometry analysis (Beckman CytoFLEX S, Beckman CytExpert 2.3.0.84, and Flow Jlo 7.6.1).

Surface Plasmon Resonance

The interaction between antibody and hACE2 is monitored by SPR using a BIAcore 8K (GE Healthcare) carried out in single-cycle mode with protein A biosensor chip (GE Healthcare). All the measurements are performed in the buffer consisting of 10 mM Na2HPO4, 2 mM KH₂PO₄, 137 mM NaCl, 2.7 mM KCl, pH 7.4, and 0.05% (v/v) Tween-20. The antibody protein is captured on the chip at ˜1000 response units. Then, gradient concentrations of ACE2 protein (from 200 to 12.5 nM with two-fold dilutions) flowed over the chip surface and the real-time response is recorded. After each cycle, the sensor is regenerated with 10 mM Gly-HCl (pH 1.5). The raw data and affinities are collected and calculated using a 1:1 fitting model with BIAevaluation software (GE Healthcare, Biacore 8 K Control Software 2.0.15.12933 and Biacore Insight Evaluation 1.0.5.11069).

hACE2 Carboxypeptidase Activity Measurement

Enzymatic reactions are performed in black microtiter plates at ambient temperature (26° C.). To each well, 25 μl of 1.6 μg/ml hACE2 (19-615) protein in PBS is added, respectively. Then, 25 μl antibody at various final concentrations of 100, 200, and 400 μg/ml or hACE2 inhibitor (MLN-4760, Sigma, 5.30616) at a final concentration of 10 μM are added to wells and incubated for 15 min. The reactions are initiated by adding 50 μl of fluorogenic peptides (Mac-APK-Dnp) (GenScript) at 40 μM or with two-fold serial dilutions ranging from 40 to 0.3125 μM to determine the kinetic constants for hACE2 hydrolysis. The relative fluorescence units (RFUs) are read at excitation and emission wavelengths of 320 and 405 nm, respectively, in kinetic mode at 2-min intervals for 6 h (BMG LABTECH. CLARIOstar Plus 5.61). To calculate the specific activity of hACE2, the intensities of RFU are converted to molarities according to standard substrate Mca-P-L-OH (GenScript), To obtain the kinetic constants, the initial velocity conditions are limited to 12 min. Initial velocities are plotted versus substrate concentration and fit to the Michaelis-Menten equation v=Vₘₐₓ[S]/(Kₘ+[S]) using GraphPad Prism software (version 6.0). Turnover numbers (k_{cat}) are calculated from the equation k_{cat}=Vₘₐₓ/[E], using the hACE2 molecular mass of 85 kDa and assuming the enzyme sample to be essentially pure and fully active.

Generation of Pseudoviruses

pcDNA3.1.S2 recombinant plasmid (GenBank: MT_613044), constructed by inserting the codon-optimized S gene of SARS-CoV-2 (GenBank: MN_908947) into pcDNA3.1, is used as the template to generate the plasmid with mutagenesis in the S gene. Following the procedure of circular PCR, 15-20 nucleotides before and after the target mutation site are selected as forward primers, while the reverse complementary sequences are selected as reverse primers. Following site-directed mutagenesis PCR, the template chain is digested using DpnI restriction endonuclease (NEB, R0176S). Afterward, the PCR product is directly used to transform Escherichia coli DH5α-competent cells (Vazyme, C502-02) and single clones are selected and then sequenced.

The SARS-CoV and SARS-CoV-2 pseudoviruses are produced using the VSV pseudovirus system as described previously. In brief, on the day before transfection, HEK293T cells are prepared and adjusted to the concentration of 5×10⁵ cell/ml, 15 ml of which are transferred into a T75 cell culture flask and incubated overnight at 37° C. in an incubator conditioned with 5% CO₂. The cells generally reach 70-90% confluence after overnight incubation. Thirty micrograms of DNA plasmid expressing the spike protein is transfected according to the user's instruction manual of Lipofectamine 3000 (Invitrogen, L3000001). The transfected cells are subsequently infected with G*ΔG-VSV (VSV G-pseudotyped virus) at concentrations of 7×10⁵ TCID50/ml. After being incubated for 6 h, the medium is replaced with a fresh medium and incubated for 24 h. The culture supernatants containing the pseudovirus are harvested, filtered (0.45 μM pore size), and stored at −80° C. TCID50 of pseudoviruses is determined as described previously.

Neutralization Assay

For pseudovirus neutralization assay, 10⁴ HEK293T-hACE2 cells per well are seeded into 96-well plates (Corning) before infection. Fifty-five microliters of three- or five-fold serially diluted antibody (from 50 μg/ml) are added to cells. After incubation at 37° C. for 1 h, 1.3×10⁴ TCID50 of SARS-CoV-2 pseudovirus in 55 μl are added in mixtures and subsequently incubated for 24 h. Transfer cell lysates (50 μl/well) are placed into luminometer plates (Microfluor 96-well plates). Add luciferase substrate (50 μl/well) is included in a luciferase assay system. The infectivity is determined by measuring the bioluminescence (Promega, GLoMax 1.9.3).

For live neutralization assay, 10⁴ Vero E6 cells per well are seeded in 96-well plates (Corning) before infection. Fifty microliters of two-fold serially diluted antibody (from 10 μg/ml) is added to Vero E6 cells with eight replicates. After incubation at 37° C. for 1 h, 100 TCID50 of SARS-CoV-2 in 50 μl is added to cells. In parallel, 10⁴ LLC-MK2 cells per well are seeded in 96-well plates (Corning) before infection. Fifty microliters of two-fold serially diluted antibody (from 100 μg/ml) is added to the cells with eight replicates. After incubation at 37° C. for 1 h, 20 TCID50 of HCoV-NL63 in 50 μl is added to the mixtures. Then, mixtures are subsequently incubated at 37 CC for 3 days. Cells infected with or without the virus are applied as positive or negative controls. CPE in each well is observed and recorded on the third day. A virus back titration is performed to assess the correct virus titer used in each experiment. AH experiments followed the standard operating procedures (SOPS) of the approved Biosafety Level-3 facility.

Mice Experiments

AH animal experiments are carried out according to the relevant procedures and relevant ethical regulations regarding animal research.

Briefly, the full cDNAs of hACE2 are knocked into the exon 2, the first coding exon, of the mAce2 gene located in GRC m38.p6 sites. hACE2 transgenic mice (female, 30 weeks old) are divided into five groups including eight mice in the placebo group injected with PBS. Animals in the pre-exposure groups are injected with 5 or 25 mg/kg antibody one day before the viral challenge. In the post-exposure groups, the mice are administered with 5 or 25 mg/kg antibody one day after the viral challenge. All mice are euthanized on the fifth day after being challenged with 5×10⁵ TCID50 of SARS-CoV-2. The lung tissues from five mice in each group are placed into 1 ml of DMEM separately. After homogenization, viral RNAs are extracted by Magnetic Bead Extraction Kit (EmerTher, RE01) according to the manufacturer's instructions and eluted in 50 μl of elution buffer and used as the template for reverse transcription-polymerase chain reaction (RT-PCR). The pairs of primers are used to target ORF1ab gene: OFR1ab-F, CCCTGTGGGTTTTACACTTAA-3′ and OFR1ab-R, 5-ACGATTGTGCATCAGCTGA-3′; Probe-ORF1ab 5′-the FAM-CCGTCTGCGGTATGTGGAAAGGTTATGG-BHQ1-3′.

Five microliters of RNA is used to verify the RNA quantity by One Step PrimeScript RT-PCR Kit (Takara, RR064B) according to the manufacturer's instructions. The amplification is performed as follows: 42° C. for 5 min, 95° C. for 10 s, followed by 40 cycles consisting of 95° C. for 3 s, 60° C. for 30 s, and a default melting-curve step in an Applied QuantStudio 5 Real-Time PCR System (QuantStudio Design and Analysis Software vi 5.1). The limit of detection in this RT-PCR program is 40 copies. When the detection is lower than 40 copies, the value is recorded as 20 copies.

Histopathology and Pathology

Mice necropsies are performed according to a standard protocol. The lung tissues of three mice in each group for histological examination are stored in 10% neutral-buffered formalin for 7 days, embedded in paraffin, sectioned, and stained with hematoxylin before examination by light microscopy.

Safety Assessment Using Cynomolgus Monkeys

Purpose-bred cynomolgus monkeys (Macaca fascicularis) are obtained from licensed vendors and undergo standard quarantine periods (˜4 weeks) before initiation. During the study periods, animals are single-housed in primary enclosures according to the appropriate regulations. AH experimental procedures (the management, sampling, and euthanasia) are conducted in appropriate facilities according to the appropriate regulations.

A total of four male cynomolgus monkeys (3 years old) are selected and randomly divided into two groups according to body weight. Cynomolgus monkeys are administered via repeated intravenous infusion (60 or 180 mg/kg at once a week for 3 weeks). During the study, the animals in each group survived until the planned euthanasia. At the end of the dosing period (D22), all animals are euthanized.

Clinical signs of toxicity are subjectively determined following standard procedures. Blood samples for hematology and clinical chemistry are drawn pre-study, D7, D14, and D21. Comprehensive hematology evaluations included determinations of differential leukocyte count and indicators of erythrocyte mass (RBC count). Meanwhile, serum chemistry analyses including the determination of serum enzyme activity are employed. Blood pressure measurements (systolic, diastolic, and mean blood pressure) are conducted on 6, 12, 24, 72, and 120 h after the completion of infusion on D8. Blood pressure (ecgAUTO v3.3.0.20).

According to the American Veterinary Medical Association principle, the amount of anesthetic is calculated based on the animal's body weight. At the end of the dosing period (D22), the animals are intramuscularly injected with 5 mg/kg Zoletil 50 (Virbac) combined with 2 mg/kg Sumianxin II (Dunhua Shengda Animal Co., Ltd). Anesthesia euthanasia is performed after femoral artery/venous release.

Statistical Analysis

Statistical significance between groups is determined by unpaired two-tailed t test. For the inhibition and neutralization experiments, IC50 and ND50 are calculated with the log (inhibitor) versus response-variable slope in GraphPad Prism 6.0. Enzyme kinetics (Kₘ and Vₘₐₓ) of ACE2 is fit with Michaelis-Menten in GraphPad Prism 6.0.

### Example 13—Antibody Expression Cassettes

Each of FIGS. 4A, 4B, and 4C shows a schematic diagram of two expression cassettes for use in two of the present rAAV vectors that together encode a four-chain embodiment of the present anti-hACE2/hTMPRSS2 bispecific antibody.

FIG. 4A shows, as one example, expression cassettes for an IgG(kih) bispecific antibody that comprises a first heavy and light chain that together bind to an epitope on hACE2 and a second heavy and light chain that together bind to an epitope on hTMPRSS2. The cassettes have the following structure: 5′ITR-CAG-Antibody Heavy Chain 1-Furin F2A-Antibody Light Chain 1-SV40 polyA-3′ITR; and 5′ITR-CAG-Antibody Heavy Chain 2-Furin F2A-Antibody Light Chain 2-SV40 polyA-3′ITR.

FIG. 4B shows, as another example, expression cassettes for an IgG(kih) bispecific antibody that comprises a first heavy chain and a common light chain that together bind to an epitope on hACE2 and a second heavy and the common light chain that together bind to an epitope on hTMPRSS2. The cassettes have the following structure: 5′ITR-CAG-Antibody Heavy Chain 1-Furin F2A-Antibody Light Chain-SV40 polyA-3′ITR; and 5′ITR-CAG-Antibody Heavy Chain 2-Furin F2A-Antibody Light Chain-SV40 polyA-3′ITR.

FIG. 4C shows, as a further example, expression cassettes for an IgG(kih) bispecific antibody that comprises a first heavy chain and a common light chain that together bind to an epitope on hACE2 and a second heavy chain that, together with the common light chain, bind to an epitope on hTMPRSS2. The cassettes have the following structure: 5′ITR-CAG-Antibody Heavy Chain 1-Furin F2A-Antibody Light Chain-SV40 polyA-3′ITR; and 5′ITR-CAG-Antibody Heavy Chain 2-SV40 polyA-3′ITR.

FIG. 4D shows a schematic diagram of a single expression cassette for inclusion in an AAV-antibody vector, wherein only one vector is needed for the expression of the present anti-hACE2/hTMPRSS2 bispecific antibody. An example of such is a tandem scFv (taFv) bispecific antibody that comprises the four antigen-binding segments Fv1 and Fv2 (that together bind to an epitope on hACE2) and Fv3 and Fv4 (that together bind to an epitope on hTMPRSS2). The cassette has the following structure: 5′ITR-CAG-Fv1, Fv2, Fv3, and Fv4 domains-SV40 polyA-3′ITR.

These cassette components include a CMV enhancer/chicken beta-actin promoter and intron (or CAG); an SV40 polyadenylation signal (or SV40 polyA); the antibody chains; and, in FIGS. 4A-4C, a furin F2A self-processing peptide cleavage site. In one embodiment, the promoter in each cassette is a liver-specific promoter. Each expression cassette is flanked by AAV serotype 2 inverted terminal repeats (ITR). In the cassette-containing bicistronic single-stranded AAV (ssAAV) vectors (FIGS. 4A-4C), both the heavy and light chains are expressed from one open reading frame using a F2A self-processing peptide from FMD. The furin cleavage sequence “RKRR” for the cellular protease furin is added for removal of amino acids left on the heavy chain C-terminus following F2A self-processing. In one embodiment of this invention, the subject rAAV vectors possess introns, and in another embodiment, they do not. Abbreviations: CMV, cytomegalovirus; SV40, simian virus 40; and FMD, foot-in-mouth disease virus.

### Example 14—rAAV Production

The subject rAAVs can be produced according to known methods. For instance, in one such method, HEK-293 cells are transfected with a select rAAV vector plasmid and two helper plasmids to allow generation of infectious AAV particles. After harvesting transfected cells and cell culture supernatant, rAAV is purified by three sequential CsCl centrifugation steps. Vector genome number is assessed by Real-Time PCR, and the purity of the preparation is verified by electron microscopy and silver-stained SDS-PAGE (Mueller, et al.).

### Example 15—Heavy and Light Chain CDR Single Point Mutation Embodiments

This example sets forth single amino acid point mutations of exemplary heavy chain CDR1, CDR2, and CDR3 regions, and exemplary light chain CDR1, CDR2, and CDR3 regions, envisioned for the hACE2-binding portion of the present bispecific antibody. These six exemplary CDR regions are those shown in FIG. 5 for humanized 11B11 VH (heavy chain) and humanized 11B11 VK (light chain), as originally presented in Supplementary FIG. 2 of Du, et al. The heavy chain CDR1 has the following amino acid sequence: GFTFIDYYMN. The heavy chain CDR2 has the following amino acid sequence: FIRNKANDYTTEYST. The heavy chain CDR3 has the following amino acid sequence: RHMYDDGFDF. The light chain CDR1 has the following amino acid sequence: ASSSVRYMH. The light chain CDR2 has the following amino acid sequence: LLIYDTSKLA. The light chain CDR3 has the following amino acid sequence: QQWSYNPLTF. For the purpose of this Example, the numbering for each CDR residue corresponds to the amino acid residue numbering in the variable region shown in FIG. 5 for humanized 11B11 VH or humanized 11B11 VK, as applicable. So, the first heavy chain CDR1 residue, G, is the 26th amino acid residue of the humanized 11B11 VH heavy chain variable region shown in FIG. 5. As such, it is referred to in this example as G26. Moreover, the amino acids used in this example are the following 20 naturally occurring amino acids: A, R, N, D, C, Q, E, G, H, I, L, K, M, F, P, S, T, W, Y, and V. So, for each of the 10 amino acid residues in the heavy chain CDR1 (beginning with G26), there are 19 point mutations possible. For instance, a point mutation whereby V replaces G26 would be written as G26V. Examples of single point mutations are set forth below for heavy chain CDR1, CDR2, and CDR3, and light chain CDR1, CDR2, and CDR3.

For heavy chain CDR1 (having the sequence GFTFIDYYMN), when the amino acid residue to be mutated is G26, the point mutants envisioned are G26A, G26R, G26N, G26D, G26C, G26Q, G26E, G26H, G26I, G26L, G26K, G26M, G26F, G26P, G26S, G26T, G26W, G26Y, and G26V. When the amino acid residue to be mutated is F27, the point mutants envisioned are F27A, F27R, F27N, F27D, F27C, F27Q, F27E, F27G, F27H, F27I, F27L, F27K, F27M, F27P, F27S, F27T, F27W, F27Y, and F27V. When the amino acid residue to be mutated is T28, the point mutants envisioned are T28A, T28R, T28N, T28D, T28C, T28Q, T28E, T28G, T28H, T28I, T28L, T28K, T28M, T28F, T28P, T28S, T28W, T28Y, and T28V. When the amino acid residue to be mutated is F29, the point mutants envisioned are F29A, F29R, F29N, F29D, F29C, F29Q, F29E, F29G, F29H, F29I, F29L, F29K, F29M, F29P, F29S, F29T, F29W, F29Y, and F29V. When the amino acid residue to be mutated is I30, the point mutants envisioned are I30A, I30R, I30N, I30D, I30C, I30Q, I30E, I30G, I30H, I30L, I30K, I30M, I30F, I30P, I30S, I30T, I30W, I30Y, and I30V. When the amino acid residue to be mutated is D31, the point mutants envisioned are D31A, D31R, D31N, D31C, D31Q, D31E, D31G, D31H, D31I, D31L, D31K, D31M, D31F, D31P, D31S, D31T, D31W, D31Y, and D31V. When the amino acid residue to be mutated is Y32, the point mutants envisioned are Y32A, Y32R, Y32N, Y32D, Y32C, Y32Q, Y32E, Y32G, Y32H, Y32I, Y32L, Y32K, Y32M, Y32F, Y32P, Y32S, Y32T, Y32W, and Y32V. When the amino acid residue to be mutated is Y33, the point mutants envisioned are Y33A, Y33R, Y33N, Y33D, Y33C, Y33Q, Y33E, Y33G, Y33H, Y33I, Y33L, Y33K, Y33M, Y33F, Y33P, Y33S, Y33T, Y33W, and Y33V. When the amino acid residue to be mutated is M34, the point mutants envisioned are M34A, M34R, M34N, M34D, M34C, M34Q, M34E, M34G, M34H, M34I, M34L, M34K, M34F, M34P, M34S, M34T, M34W, M34Y, and M34V. When the amino acid residue to be mutated is N35, the point mutants envisioned are N35A, N35R, N35D, N35C, N35Q, N35E, N35G, N35H, N35I, N35L, N35K, N35M, N35F, N35P, N35S, N35T, N35W, N35Y, and N35V.

For heavy chain CDR2 (having the sequence FIRNKANDYTTEYST), when the amino acid residue to be mutated is F50, the point mutants envisioned are F50A, F50R, F50N, F50D, F50C, F50Q, F50E, F50G, F50H, F50I, F50L, F50K, F50M, F50P, F50S, F50T, F50W, F50Y, and F50V. When the amino acid residue to be mutated is I51, the point mutants envisioned are I51A, I51R, I51N, I51D, I51C, I51Q, I51E, I51G, I51H, I51L, I51K, I51M, I51F, I51P, I51S, I51T, I51W, I51Y, and I51V. When the amino acid residue to be mutated is R52, the point mutants envisioned are R52A, R52N, R52D, R52C, R52Q, R52E, R52G, R52H, R52I, R52L, R52K, R52M, R52F, R52P, R52S, R52T, R52W, R52Y, and R52V. When the amino acid residue to be mutated is N53, the point mutants envisioned are N53A, N53R, N53D, N53C, N53Q, N53E, N53G, N53H, N53I, N53L, N53K, N53M, N53F, N53P, N53S, N53T, N53W, N53Y, and N53V. When the amino acid residue to be mutated is K54, the point mutants envisioned are K54A, K54R, K54N, K54D, K54C, K54Q, K54E, K54G, K54H, K54I, K54L, K54M, K54F, K54P, K54S, K54T, K54W, K54Y, and K54V. When the amino acid residue to be mutated is A55, the point mutants envisioned are A55R, A55N, A55D, A55C, A55Q, A55E, A55G, A55H, A55I, A55L, A55K, A55M, A55F, A55P, A55S, A55T, A55W, A55Y, and A55V. When the amino acid residue to be mutated is N56, the point mutants envisioned are N56A, N56R, N56D, N56C, N56Q, N56E, N56G, N56H, N56I, N56L, N56K, N56M, N56F, N56P, N56S, N56T, N56W, N56Y, and N56V. When the amino acid residue to be mutated is D57, the point mutants envisioned are D57A, D57R, D57N, D57C, D57Q, D57E, D57G, D57H, D57I, D57L, D57K, D57M, D57F, D57P, D57S, D57T, D57W, D57Y, and D57V. When the amino acid residue to be mutated is Y58, the point mutants envisioned are Y58A, Y58R, Y58N, Y58D, Y58C, Y58Q, Y58E, Y58G, Y58H, Y58I, Y58L, Y58K, Y58M, Y58F, Y58P, Y58S, Y58T, Y58W, and Y58V. When the amino acid residue to be mutated is T59, the point mutants envisioned are T59A, T59R, T59N, T59D, T59C, T59Q, T59E, T59G, T59H, T59I, T59L, T59K, T59M, T59F, T59P, T59S, T59W, T59Y, and T59V. When the amino acid residue to be mutated is T60, the point mutants envisioned are T60A, T60R, T60N, T60D, T60C, T60Q, T60E, T60G, T60H, T60I, T60L, T60K, T60M, T60F, T60P, T60S, T60W, T60Y, and T60V. When the amino acid residue to be mutated is E61, the point mutants envisioned are E61A, E61R, E61N, E61D, E61C, E61Q, E61E, E61G, E61H, E61I, E61L, E61K, E61M, E61F, E61P, E61S, E61T, E61W, E61Y, and E61V. When the amino acid residue to be mutated is Y62, the point mutants envisioned are Y62A, Y62R, Y62N, Y62D, Y62C, Y62Q, Y62E, Y62G, Y62H, Y62I, Y62L, Y62K, Y62M, Y62F, Y62P, Y62S, Y62T, Y62W, Y62Y, and Y62V. When the amino acid residue to be mutated is S63, the point mutants envisioned are S63A, S63R, S63N, S63D, S63C, S63Q, S63E, S63G, S63H, S63I, S63L, S63K, S63M, S63F, S63P, S63S, S63T, S63W, S63Y, and S63V. When the amino acid residue to be mutated is T64, the point mutants envisioned are T64A, T64R, T64N, T64D, T64C, T64Q, T64E, T64G, T64H, T64I, T64L, T64K, T64M, T64F, T64P, T64S, T64T, T64W, T64Y, and T64V.

For heavy chain CDR3 (having the sequence RHMYDDGFDF), when the amino acid residue to be mutated is R93, the point mutants envisioned are R93A, R93N, R93D, R93C, R93Q, R93E, R93G, R93H, R93I, R93L, R93K, R93M, R93F, R93P, R93S, R93T, R93W, R93Y, and R93V. When the amino acid residue to be mutated is H94, the point mutants envisioned are H94A, H94R, H94N, H94D, H94C, H94Q, H94E, H94G, H94I, H94L, H94K, H94M, H94F, H94P, H94S, H94T, H94W, H94Y, and H94V. When the amino acid residue to be mutated is M95, the point mutants envisioned are M95A, M95R, M95N, M95D, M95C, M95Q, M95E, M95G, M95H, M95I, M95L, M95K, M95F, M95P, M95S, M95T, M95W, M95Y, and M95V. When the amino acid residue to be mutated is Y96, the point mutants envisioned are Y96A, Y96R, Y96N, Y96D, Y96C, Y96Q, Y96E, Y96G, Y96H, Y96I, Y96L, Y96K, Y96M, Y96F, Y96P, Y96S, Y96T, Y96W, and Y96V. When the amino acid residue to be mutated is D97, the point mutants envisioned are D97A, D97R, D97N, D97C, D97Q, D97E, D97G, D97H, D97I, D97L, D97K, D97M, D97F, D97P, D97S, D97T, D97W, D97Y, and D97V. When the amino acid residue to be mutated is D98, the point mutants envisioned are D98A, D98R, D98N, D98C, D98Q, D98E, D98G, D98H, D98I, D98L, D98K, D98M, D98F, D98P, D98S, D98T, D98W, D98Y, and D98V. When the amino acid residue to be mutated is G99, the point mutants envisioned are G99A, G99R, G99N, G99D, G99C, G99Q, G99E, G99H, G99I, G99L, G99K, G99M, G99F, G99P, G99S, G99T, G99W, G99Y, and G99V. When the amino acid residue to be mutated is F100, the point mutants envisioned are F100A, F100R, F100N, F100D, F100C, F100Q, F100E, F100G, F100H, F100I, F100L, F100K, F100M, F100P, F100S, F100T, F100W, F100Y, and F100V. When the amino acid residue to be mutated is D101, the point mutants envisioned are D101A, D101R, D101N, D101C, D101Q, D101E, D101G, D101H, D101I, D101L, D101K, D101M, D101F, D101P, D101S, D101T, D101W, D101Y, and D101V. When the amino acid residue to be mutated is F102, the point mutants envisioned are F102A, F102R, F102N, F102D, F102C, F102Q, F102E, F102G, F102H, F102I, F102L, F102K, F102M, F102P, F102S, F102T, F102W, F102Y, and F102V.

For light chain CDR1 (having the sequence ASSSVRYMH, wherein R30 is immediately followed by Y32), when the amino acid residue to be mutated is A25, the point mutants envisioned are A25R, A25N, A25D, A25C, A25Q, A25E, A25G, A25H, A25I, A25L, A25K, A25M, A25F, A25P, A25S, A25T, A25W, A25Y, and A25V. When the amino acid residue to be mutated is S26, the point mutants envisioned are S26A, S26R, S26N, S26D, S26C, S26Q, S26E, S26G, S26H, S26I, S26L, S26K, S26M, S26F, S26P, S26T, S26W, S26Y, and S26V. When the amino acid residue to be mutated is S27, the point mutants envisioned are S27A, S27R, S27N, S27D, S27C, S27Q, S27E, S27G, S27H, S27I, S27L, S27K, S27M, S27F, S27P, S27T, S27W, S27Y, and S27V. When the amino acid residue to be mutated is S28, the point mutants envisioned are S28A, S28R, S28N, S28D, S28C, S28Q, S28E, S28G, S28H, S28I, S28L, S28K, S28M, S28F, S28P, S28T, S28W, S28Y, and S28V. When the amino acid residue to be mutated is V29, the point mutants envisioned are V29A, V29R, V29N, V29D, V29C, V29Q, V29E, V29G, V29H, V29I, V29L, V29K, V29M, V29F, V29P, V29S, V29T, V29W, and V29Y. When the amino acid residue to be mutated is R30, the point mutants envisioned are R30A, R30N, R30D, R30C, R30Q, R30E, R30G, R30H, R30I, R30L, R30K, R30M, R30F, R30P, R30S, R30T, R30W, R30Y, and R30V. When the amino acid residue to be mutated is Y32, the point mutants envisioned are Y32A, Y32R, Y32N, Y32D, Y32C, Y32Q, Y32E, Y32G, Y32H, Y32I, Y32L, Y32K, Y32M, Y32F, Y32P, Y32S, Y32T, Y32W, and Y32V. When the amino acid residue to be mutated is M33, the point mutants envisioned are M33A, M33R, M33N, M33D, M33C, M33Q, M33E, M33G, M33H, M33I, M33L, M33K, M33F, M33P, M33S, M33T, M33W, M33Y, and M33V. When the amino acid residue to be mutated is H34, the point mutants envisioned are H34A, H34R, H34N, H34D, H34C, H34Q, H34E, H34G, H34I, H34L, H34K, H34M, H34F, H34P, H34S, H34T, H34W, H34Y, and H34V.

For light chain CDR2 (having the sequence LLIYDTSKLA), when the amino acid residue to be mutated is L46, the point mutants envisioned are L46A, L46R, L46N, L46D, L46C, L46Q, L46E, L46G, L46H, L46I, L46K, L46M, L46F, L46P, L46S, L46T, L46W, L46Y, and L46V. When the amino acid residue to be mutated is L47, the point mutants envisioned are L47A, L47R, L47N, L47D, L47C, L47Q, L47E, L47G, L47H, L47I, L47K, L47M, L47F, L47P, L47S, L47T, L47W, L47Y, and L47V. When the amino acid residue to be mutated is I48, the point mutants envisioned are I48A, I48R, I48N, I48D, I48C, I48Q, I48E, I48G, I48H, I48L, I48K, I48M, I48F, I48P, I48S, I48T, I48W, I48Y, and I48V. When the amino acid residue to be mutated is Y49, the point mutants envisioned are Y49A, Y49R, Y49N, Y49D, Y49C, Y49Q, Y49E, Y49G, Y49H, Y49I, Y49L, Y49K, Y49M, Y49F, Y49P, Y49S, Y49T, Y49W, and Y49V. When the amino acid residue to be mutated is D50, the point mutants envisioned are D50A, D50R, D50N, D50C, D50Q, D50E, D50G, D50H, D50I, D50L, D50K, D50M, D50F, D50P, D50S, D50T, D50W, D50Y, and D50V. When the amino acid residue to be mutated is T51, the point mutants envisioned are T51A, T51R, T51N, T51D, T51C, T51Q, T51E, T51G, T51H, T51I, T51L, T51K, T51M, T51F, T51P, T51S, T51W, T51Y, and T51V. When the amino acid residue to be mutated is S52, the point mutants envisioned are S52A, S52R, S52N, S52D, S52C, S52Q, S52E, S52G, S52H, S52I, S52L, S52K, S52M, S52F, S52P, S52T, S52W, S52Y, and S52V. When the amino acid residue to be mutated is K53, the point mutants envisioned are K53A, K53R, K53N, K53D, K53C, K53Q, K53E, K53G, K53H, K53I, K53L, K53M, K53F, K53P, K53S, K53T, K53W, K53Y, and K53V. When the amino acid residue to be mutated is L54, the point mutants envisioned are L54A, L54R, L54N, L54D, L54C, L54Q, L54E, L54G, L54H, L54I, L54K, L54M, L54F, L54P, L54S, L54T, L54W, L54Y, and L54V. When the amino acid residue to be mutated is A55, the point mutants envisioned are A55R, A55N, A55D, A55C, A55Q, A55E, A55G, A55H, A55I, A55L, A55K, A55M, A55F, A55P, A55S, A55T, A55W, A55Y, and A55V.

For light chain CDR3 (having the sequence QQWSYNPLTF), when the amino acid residue to be mutated is Q89, the point mutants envisioned are Q89A, Q89R, Q89N, Q89D, Q89C, Q89E, Q89G, Q89H, Q89I, Q89L, Q89K, Q89M, Q89F, Q89P, Q89S, Q89T, Q89W, Q89Y, and Q89V. When the amino acid residue to be mutated is Q90, the point mutants envisioned are Q90A, Q90R, Q90N, Q90D, Q90C, Q90E, Q90G, Q90H, Q90I, Q90L, Q90K, Q90M, Q90F, Q90P, Q90S, Q90T, Q90W, Q90Y, and Q90V. When the amino acid residue to be mutated is W91, the point mutants envisioned are W91A, W91R, W91N, W91D, W91C, W91Q, W91E, W91G, W91H, W91I, W91L, W91K, W91M, W91F, W91P, W91S, W91T, W91Y, and W91V. When the amino acid residue to be mutated is S92, the point mutants envisioned are S92A, S92R, S92N, S92D, S92C, S92Q, S92E, S92G, S92H, S92I, S92L, S92K, S92M, S92F, S92P, S92T, S92W, S92Y, and S92V. When the amino acid residue to be mutated is Y93, the point mutants envisioned are Y93A, Y93R, Y93N, Y93D, Y93C, Y93Q, Y93E, Y93G, Y93H, Y93I, Y93L, Y93K, Y93M, Y93F, Y93P, Y93S, Y93T, Y93W, and Y93V. When the amino acid residue to be mutated is N94, the point mutants envisioned are N94A, N94R, N94D, N94C, N94Q, N94E, N94G, N94H, N94I, N94L, N94K, N94M, N94F, N94P, N94S, N94T, N94W, N94Y, and N94V. When the amino acid residue to be mutated is P95, the point mutants envisioned are P95A, P95R, P95N, P95D, P95C, P95Q, P95E, P95G, P95H, P95I, P95L, P95K, P95M, P95F, P95S, P95T, P95W, P95Y, and P95V. When the amino acid residue to be mutated is L96, the point mutants envisioned are L96A, L96R, L96N, L96D, L96C, L96Q, L96E, L96G, L96H, L96I, L96K, L96M, L96F, L96P, L96S, L96T, L96W, L96Y, and L96V. When the amino acid residue to be mutated is T97, the point mutants envisioned are T97A, T97R, T97N, T97D, T97C, T97Q, T97E, T97G, T97H, T97I, T97L, T97K, T97M, T97F, T97P, T97S, T97W, T97Y, and T97V. When the amino acid residue to be mutated is F98, the point mutants envisioned are F98A, F98R, F98N, F98D, F98C, F98Q, F98E, F98G, F98H, F98I, F98L, F98K, F98M, F98P, F98S, F98T, F98W, F98Y, and F98V.

### Example 16—Heavy Chain CDR3 Double Point Mutation Embodiments

This example sets forth examples of double amino acid point mutations of an exemplary heavy chain CDR3 envisioned for the hACE2-binding portion of the present bispecific antibody. Again, the heavy chain CDR3 has the following amino acid sequence: RHMYDDGFDF, wherein the numbering for each heavy chain CDR3 residue corresponds to the amino acid residue numbering in the heavy chain variable region shown in FIG. 5. So, for example, the first and third heavy chain CDR3 residues, i.e., R and M, are, respectively, the 93rd and 95th amino acid residues of the heavy chain variable region shown in FIG. 5. As such, they are referred to in this example as R93 and M95. As in Example 15, the amino acids used in this example are the following 20 naturally occurring amino acids: A, R, N, D, C, Q, E, G, H, I, L, K, M, F, P, S, T, W, Y, and V. And again, for each of the 10 amino acid residues in the heavy chain CDR3 (beginning with R93), there are 19 single point mutations possible. For each double point mutation, however, there are far more permutations possible. For example, R93A/M95Q (i.e., the double point mutation wherein A replaces R93 and Q replaces M95) would constitute one of the many double point mutations possible. Examples of double point mutations are set forth below. In each example, the double point mutation is expressed as a two-letter abbreviation. So, for example, the double point mutation R93A/M95Q would be expressed simply as AQ.

When the first and second amino acid residues to be mutated are R93 and H94, the double point mutants envisioned are as follows:

AA, AR, AN, AD, AC, AQ, AE, AG, AI, AL, AK, AM, AF, AP, AS, AT, AW, AY, AV, NA, NR, NN, ND, NC, NQ, NE, NG, NI, NL, NK, NM, NF, NP, NS, NT, NW, NY, NV, DA, DR, DN, DD, DC, DQ, DE, DG, DI, DL, DK, DM, DF, DP, DS, DT, DW, DY, DV, CA, CR, CN, CD, CC, CQ, CE, CG, CI, CL, CK, CM, CF, CP, CS, CT, CW, CY, CV, QA, QR, QN, QD, QC, QQ, QE, QG, QI, QL, QK, QM, QF, QP, QS, QT, QW, QY, QV, EA, ER, EN, ED, EC, EQ, EE, EG, EI, EL, EK, EM, EF, EP, ES, ET, EW, EY, EV, GA, GR, GN, GD, GC, GQ, GE, GG, GI, GL, GK, GM, GF, GP, GS, GT, GW, GY, GV, HA, HR, HN, HD, HC, HQ, HE, HG, HI, HL, HK, HM, HF, HP, HS, HT, HW, HY, HV, IA, IR, IN, ID, IC, IQ, IE, IG, II, IL, IK, IM, IF, IP, IS, IT, IW, IY, IV, LA, LR, LN, LD, LC, LQ, LE, LG, LI, LL, LK, LM, LF, LP, LS, LT, LW, LY, LV, KA, KR, KN, KD, KC, KQ, KE, KG, KI, KL, KK, KM, KF, KP, KS, KT, KW, KY, KV, MA, MR, MN, MD, MC, MQ, ME, MG, MI, ML, MK, MM, MF, MP, MS, MT, MW, MY, MV, FA, FR, FN, FD, FC, FQ, FE, FG, FI, FL, FK, FM, FF, FP, FS, FT, FW, FY, FV, PA, PR, PN, PD, PC, PQ, PE, PG, PI, PL, PK, PM, PF, PP, PS, PT, PW, PY, PV, SA, SR, SN, SD, SC, SQ, SE, SG, SI, SL, SK, SM, SF, SP, SS, ST, SW, SY, SV, TA, TR, TN, TD, TC, TQ, TE, TG, TI, TL, TK, TM, TF, TP, TS, TT, TW, TY, TV, WA, WR, WN, WD, WC, WQ, WE, WG, WI, WL, WK, WM, WF, WP, WS, WT, WW, WY, WV, YA, YR, YN, YD, YC, YQ, YE, YG, YI, YL, YK, YM, YF, YP, YS, YT, YW, YY, YV, VA, VR, VN, VD, VC, VQ, VE, VG, VI, VL, VK, VM, VF, VP, VS, VT, VW, VY, and W.

When the first and second amino acid residues to be mutated are R93 and M95, the double point mutants envisioned are as follows:

AA, AR, AN, AD, AC, AQ, AE, AG, AH, AI, AL, AK, AF, AP, AS, AT, AW, AY, AV, NA, NR, NN, ND, NC, NQ, NE, NG, NH, NI, NL, NK, NF, NP, NS, NT, NW, NY, NV, DA, DR, DN, DD, DC, DQ, DE, DG, DH, DI, DL, DK, DF, DP, DS, DT, DW, DY, DV, CA, CR, CN, CD, CC, CQ, CE, CG, CH, CI, CL, CK, CF, CP, CS, CT, CW, CY, CV, QA, QR, QN, QD, QC, QQ, QE, QG, QH, QI, QL, QK, QF, QP, QS, QT, QW, QY, QV, EA, ER, EN, ED, EC, EQ, EE, EG, EH, EI, EL, EK, EF, EP, ES, ET, EW, EY, EV, GA, GR, GN, GD, GC, GQ, GE, GG, GH, GI, GL, GK, GF, GP, GS, GT, GW, GY, GV, HA, HR, HN, HD, HC, HQ, HE, HG, HH, HI, HL, HK, HF, HP, HS, HT, HW, HY, HV, IA, IR, IN, ID, IC, IQ, IE, IG, IH, II, IL, IK, IF, IP, IS, IT, IW, IY, IV, LA, LR, LN, LD, LC, LQ, LE, LG, LH, LI, LL, LK, LF, LP, LS, LT, LW, LY, LV, KA, KR, KN, KD, KC, KQ, KE, KG, KH, KI, KL, KK, KF, KP, KS, KT, KW, KY, KV, MA, MR, MN, MD, MC, MQ, ME, MG, MH, MI, ML, MK, MF, MP, MS, MT, MW, MY, MV, FA, FR, FN, FD, FC, FQ, FE, FG, FH, FI, FL, FK, FF, FP, FS, FT, FW, FY, FV, PA, PR, PN, PD, PC, PQ, PE, PG, PH, PI, PL, PK, PF, PP, PS, PT, PW, PY, PV, SA, SR, SN, SD, SC, SQ, SE, SG, SH, SI, SL, SK, SF, SP, SS, ST, SW, SY, SV, TA, TR, TN, TD, TC, TQ, TE, TG, TH, TI, TL, TK, TF, TP, TS, TT, TW, TY, TV, WA, WR, WN, WD, WC, WQ, WE, WG, WH, WI, WL, WK, WF, WP, WS, WT, WW, WY, WV, YA, YR, YN, YD, YC, YQ, YE, YG, YH, YI, YL, YK, YF, YP, YS, YT, YW, YY, YV, VA, VR, VN, VD, VC, VQ, VE, VG, VH, VI, VL, VK, VF, VP, VS, VT, VW, VY, and W.

When the first and second amino acid residues to be mutated are R93 and Y96, the double point mutants envisioned are as follows:

AA, AR, AN, AD, AC, AQ, AE, AG, AH, AI, AL, AK, AM, AF, AP, AS, AT, AW, AV, NA, NR, NN, ND, NC, NQ, NE, NG, NH, NI, NL, NK, NM, NF, NP, NS, NT, NW, NV, DA, DR, DN, DD, DC, DQ, DE, DG, DH, DI, DL, DK, DM, DF, DP, DS, DT, DW, DV, CA, CR, CN, CD, CC, CQ, CE, CG, CH, CI, CL, CK, CM, CF, CP, CS, CT, CW, CV, QA, QR, QN, QD, QC, QQ, QE, QG, QH, QI, QL, QK, QM, QF, QP, QS, QT, QW, QV, EA, ER, EN, ED, EC, EQ, EE, EG, EH, EI, EL, EK, EM, EF, EP, ES, ET, EW, EV, GA, GR, GN, GD, GC, GQ, GE, GG, GH, GI, GL, GK, GM, GF, GP, GS, GT, GW, GV, HA, HR, HN, HD, HC, HQ, HE, HG, HH, HI, HL, HK, HM, HF, HP, HS, HT, HW, HV, IA, IR, IN, ID, IC, IQ, IE, IG, IH, II, IL, IK, IM, IF, IP, IS, IT, IW, IV, LA, LR, LN, LD, LC, LQ, LE, LG, LH, LI, LL, LK, LM, LF, LP, LS, LT, LW, LV, KA, KR, KN, KD, KC, KQ, KE, KG, KH, KI, KL, KK, KM, KF, KP, KS, KT, KW, KV, MA, MR, MN, MD, MC, MQ, ME, MG, MH, MI, ML, MK, MM, MF, MP, MS, MT, MW, MV, FA, FR, FN, FD, FC, FQ, FE, FG, FH, FI, FL, FK, FM, FF, FP, FS, FT, FW, FV, PA, PR, PN, PD, PC, PQ, PE, PG, PH, PI, PL, PK, PM, PF, PP, PS, PT, PW, PV, SA, SR, SN, SD, SC, SQ, SE, SG, SH, SI, SL, SK, SM, SF, SP, SS, ST, SW, SV, TA, TR, TN, TD, TC, TQ, TE, TG, TH, TI, TL, TK, TM, TF, TP, TS, TT, TW, TV, WA, WR, WN, WD, WC, WQ, WE, WG, WH, WI, WL, WK, WM, WF, WP, WS, WT, WW, WV, YA, YR, YN, YD, YC, YQ, YE, YG, YH, YI, YL, YK, YM, YF, YP, YS, YT, YW, YV, VA, VR, VN, VD, VC, VQ, VE, VG, VH, VI, VL, VK, VM, VF, VP, VS, VT, VW, and W.

When the first and second amino acid residues to be mutated are R93 and D97, the double point mutants envisioned are as follows:

AA, AR, AN, AC, AQ, AE, AG, AH, AI, AL, AK, AM, AF, AP, AS, AT, AW, AY, AV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and W.

When the first and second amino acid residues to be mutated are R93 and D98, the double point mutants envisioned are as follows:

AA, AR, AN, AC, AQ, AE, AG, AH, AI, AL, AK, AM, AF, AP, AS, AT, AW, AY, AV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and W.

When the first and second amino acid residues to be mutated are R93 and G99, the double point mutants envisioned are as follows:

AA, AR, AN, AD, AC, AQ, AE, AH, AI, AL, AK, AM, AF, AP, AS, AT, AW, AY, AV, NA, NR, NN, ND, NC, NQ, NE, NH, NI, NL, NK, NM, NF, NP, NS, NT, NW, NY, NV, DA, DR, DN, DD, DC, DQ, DE, DH, DI, DL, DK, DM, DF, DP, DS, DT, DW, DY, DV, CA, CR, CN, CD, CC, CQ, CE, CH, CI, CL, CK, CM, CF, CP, CS, CT, CW, CY, CV, QA, QR, QN, QD, QC, QQ, QE, QH, QI, QL, QK, QM, QF, QP, QS, QT, QW, QY, QV, EA, ER, EN, ED, EC, EQ, EE, EH, EI, EL, EK, EM, EF, EP, ES, ET, EW, EY, EV, GA, GR, GN, GD, GC, GQ, GE, GH, GI, GL, GK, GM, GF, GP, GS, GT, GW, GY, GV, HA, HR, HN, HD, HC, HQ, HE, HH, HI, HL, HK, HM, HF, HP, HS, HT, HW, HY, HV, IA, IR, IN, ID, IC, IQ, IE, IH, II, IL, IK, IM, IF, IP, IS, IT, IW, IY, IV, LA, LR, LN, LD, LC, LQ, LE, LH, LI, LL, LK, LM, LF, LP, LS, LT, LW, LY, LV, KA, KR, KN, KD, KC, KQ, KE, KH, KI, KL, KK, KM, KF, KP, KS, KT, KW, KY, KV, MA, MR, MN, MD, MC, MQ, ME, MH, MI, ML, MK, MM, MF, MP, MS, MT, MW, MY, MV, FA, FR, FN, FD, FC, FQ, FE, FH, FI, FL, FK, FM, FF, FP, FS, FT, FW, FY, FV, PA, PR, PN, PD, PC, PQ, PE, PH, PI, PL, PK, PM, PF, PP, PS, PT, PW, PY, PV, SA, SR, SN, SD, SC, SQ, SE, SH, SI, SL, SK, SM, SF, SP, SS, ST, SW, SY, SV, TA, TR, TN, TD, TC, TQ, TE, TH, TI, TL, TK, TM, TF, TP, TS, TT, TW, TY, TV, WA, WR, WN, WD, WC, WQ, WE, WH, WI, WL, WK, WM, WF, WP, WS, WT, WW, WY, WV, YA, YR, YN, YD, YC, YQ, YE, YH, YI, YL, YK, YM, YF, YP, YS, YT, YW, YY, YV, VA, VR, VN, VD, VC, VQ, VE, VH, VI, VL, VK, VM, VF, VP, VS, VT, VW, VY, and W.

When the first and second amino acid residues to be mutated are R93 and F100, the double point mutants envisioned are as follows:

AA, AR, AN, AD, AC, AQ, AE, AG, AH, AI, AL, AK, AM, AP, AS, AT, AW, AY, AV, NA, NR, NN, ND, NC, NQ, NE, NG, NH, NI, NL, NK, NM, NP, NS, NT, NW, NY, NV, DA, DR, DN, DD, DC, DQ, DE, DG, DH, DI, DL, DK, DM, DP, DS, DT, DW, DY, DV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are R93 and D101, the double point mutants envisioned are as follows:

AA, AR, AN, AC, AQ, AE, AG, AH, AI, AL, AK, AM, AF, AP, AS, AT, AW, AY, AV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and W.

When the first and second amino acid residues to be mutated are R93 and F102, the double point mutants envisioned are as follows:

AA, AR, AN, AD, AC, AQ, AE, AG, AH, AI, AL, AK, AM, AP, AS, AT, AW, AY, AV, NA, NR, NN, ND, NC, NQ, NE, NG, NH, NI, NL, NK, NM, NP, NS, NT, NW, NY, NV, DA, DR, DN, DD, DC, DQ, DE, DG, DH, DI, DL, DK, DM, DP, DS, DT, DW, DY, DV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are H94 and M95, the double point mutants envisioned are as follows:

AA, AR, AN, AD, AC, AQ, AE, AG, AH, AI, AL, AK, AF, AP, AS, AT, AW, AY, AV, RA, RR, RN, RD, RC, RQ, RE, RG, RH, RI, RL, RK, RF, RP, RS, RT, RW, RY, RV, NA, NR, NN, ND, NC, NQ, NE, NG, NH, NI, NL, NK, NF, NP, NS, NT, NW, NY, NV, DA, DR, DN, DD, DC, DQ, DE, DG, DH, DI, DL, DK, DF, DP, DS, DT, DW, DY, DV, CA, CR, CN, CD, CC, CQ, CE, CG, CH, CI, CL, CK, CF, CP, CS, CT, CW, CY, CV, QA, QR, QN, QD, QC, QQ, QE, QG, QH, QI, QL, QK, QF, QP, QS, QT, QW, QY, QV, EA, ER, EN, ED, EC, EQ, EE, EG, EH, EI, EL, EK, EF, EP, ES, ET, EW, EY, EV, GA, GR, GN, GD, GC, GQ, GE, GG, GH, GI, GL, GK, GF, GP, GS, GT, GW, GY, GV, IA, IR, IN, ID, IC, IQ, IE, IG, IH, II, IL, IK, IF, IP, IS, IT, IW, IY, IV, LA, LR, LN, LD, LC, LQ, LE, LG, LH, LI, LL, LK, LF, LP, LS, LT, LW, LY, LV, KA, KR, KN, KD, KC, KQ, KE, KG, KH, KI, KL, KK, KF, KP, KS, KT, KW, KY, KV, MA, MR, MN, MD, MC, MQ, ME, MG, MH, MI, ML, MK, MF, MP, MS, MT, MW, MY, MV, FA, FR, FN, FD, FC, FQ, FE, FG, FH, FI, FL, FK, FF, FP, FS, FT, FW, FY, FV, PA, PR, PN, PD, PC, PQ, PE, PG, PH, PI, PL, PK, PF, PP, PS, PT, PW, PY, PV, SA, SR, SN, SD, SC, SQ, SE, SG, SH, SI, SL, SK, SF, SP, SS, ST, SW, SY, SV, TA, TR, TN, TD, TC, TQ, TE, TG, TH, TI, TL, TK, TF, TP, TS, TT, TW, TY, TV, WA, WR, WN, WD, WC, WQ, WE, WG, WH, WI, WL, WK, WF, WP, WS, WT, WW, WY, WV, YA, YR, YN, YD, YC, YQ, YE, YG, YH, YI, YL, YK, YF, YP, YS, YT, YW, YY, YV, VA, VR, VN, VD, VC, VQ, VE, VG, VH, VI, VL, VK, VF, VP, VS, VT, VW, VY, and W.

When the first and second amino acid residues to be mutated are H94 and Y96, the double point mutants envisioned are as follows:

AA, AR, AN, AD, AC, AQ, AE, AG, AH, AI, AL, AK, AM, AF, AP, AS, AT, AW, AV, RA, RR, RN, RD, RC, RQ, RE, RG, RH, RI, RL, RK, RM, RF, RP, RS, RT, RW, RV, NA, NR, NN, ND, NC, NQ, NE, NG, NH, NI, NL, NK, NM, NF, NP, NS, NT, NW, NV, DA, DR, DN, DD, DC, DQ, DE, DG, DH, DI, DL, DK, DM, DF, DP, DS, DT, DW, DV, CA, CR, CN, CD, CC, CQ, CE, CG, CH, CI, CL, CK, CM, CF, CP, CS, CT, CW, CV, QA, QR, QN, QD, QC, QQ, QE, QG, QH, QI, QL, QK, QM, QF, QP, QS, QT, QW, QV, EA, ER, EN, ED, EC, EQ, EE, EG, EH, EI, EL, EK, EM, EF, EP, ES, ET, EW, EV, GA, GR, GN, GD, GC, GQ, GE, GG, GH, GI, GL, GK, GM, GF, GP, GS, GT, GW, GV, IA, IR, IN, ID, IC, IQ, IE, IG, IH, II, IL, IK, IM, IF, IP, IS, IT, IW, IV, LA, LR, LN, LD, LC, LQ, LE, LG, LH, LI, LL, LK, LM, LF, LP, LS, LT, LW, LV, KA, KR, KN, KD, KC, KQ, KE, KG, KH, KI, KL, KK, KM, KF, KP, KS, KT, KW, KV, MA, MR, MN, MD, MC, MQ, ME, MG, MH, MI, ML, MK, MM, MF, MP, MS, MT, MW, MV, FA, FR, FN, FD, FC, FQ, FE, FG, FH, FI, FL, FK, FM, FF, FP, FS, FT, FW, FV, PA, PR, PN, PD, PC, PQ, PE, PG, PH, PI, PL, PK, PM, PF, PP, PS, PT, PW, PV, SA, SR, SN, SD, SC, SQ, SE, SG, SH, SI, SL, SK, SM, SF, SP, SS, ST, SW, SV, TA, TR, TN, TD, TC, TQ, TE, TG, TH, TI, TL, TK, TM, TF, TP, TS, TT, TW, TV, WA, WR, WN, WD, WC, WQ, WE, WG, WH, WI, WL, WK, WM, WF, WP, WS, WT, WW, WV, YA, YR, YN, YD, YC, YQ, YE, YG, YH, YI, YL, YK, YM, YF, YP, YS, YT, YW, YV, VA, VR, VN, VD, VC, VQ, VE, VG, VH, VI, VL, VK, VM, VF, VP, VS, VT, VW, and W.

When the first and second amino acid residues to be mutated are H94 and D97, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and VV.

When the first and second amino acid residues to be mutated are H94 and D98, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and VV.

When the first and second amino acid residues to be mutated are H94 and G99, the double point mutants envisioned are as follows:

AA, AR, AN, AD, AC, AQ, AE, AH, AI, AL, AK, AM, AF, AP, AS, AT, AW, AY, AV, RA, RR, RN, RD, RC, RQ, RE, RH, RI, RL, RK, RM, RF, RP, RS, RT, RW, RY, RV, NA, NR, NN, ND, NC, NQ, NE, NH, NI, NL, NK, NM, NF, NP, NS, NT, NW, NY, NV, DA, DR, DN, DD, DC, DQ, DE, DH, DI, DL, DK, DM, DF, DP, DS, DT, DW, DY, DV, CA, CR, CN, CD, CC, CQ, CE, CH, CI, CL, CK, CM, CF, CP, CS, CT, CW, CY, CV, QA, QR, QN, QD, QC, QQ, QE, QH, QI, QL, QK, QM, QF, QP, QS, QT, QW, QY, QV, EA, ER, EN, ED, EC, EQ, EE, EH, EI, EL, EK, EM, EF, EP, ES, ET, EW, EY, EV, GA, GR, GN, GD, GC, GQ, GE, GH, GI, GL, GK, GM, GF, GP, GS, GT, GW, GY, GV, IA, IR, IN, ID, IC, IQ, IE, IH, II, IL, IK, IM, IF, IP, IS, IT, IW, IY, IV, LA, LR, LN, LD, LC, LQ, LE, LH, LI, LL, LK, LM, LF, LP, LS, LT, LW, LY, LV, KA, KR, KN, KD, KC, KQ, KE, KH, KI, KL, KK, KM, KF, KP, KS, KT, KW, KY, KV, MA, MR, MN, MD, MC, MQ, ME, MH, MI, ML, MK, MM, MF, MP, MS, MT, MW, MY, MV, FA, FR, FN, FD, FC, FQ, FE, FH, FI, FL, FK, FM, FF, FP, FS, FT, FW, FY, FV, PA, PR, PN, PD, PC, PQ, PE, PH, PI, PL, PK, PM, PF, PP, PS, PT, PW, PY, PV, SA, SR, SN, SD, SC, SQ, SE, SH, SI, SL, SK, SM, SF, SP, SS, ST, SW, SY, SV, TA, TR, TN, TD, TC, TQ, TE, TH, TI, TL, TK, TM, TF, TP, TS, TT, TW, TY, TV, WA, WR, WN, WD, WC, WQ, WE, WH, WI, WL, WK, WM, WF, WP, WS, WT, WW, WY, WV, YA, YR, YN, YD, YC, YQ, YE, YH, YI, YL, YK, YM, YF, YP, YS, YT, YW, YY, YV, VA, VR, VN, VD, VC, VQ, VE, VH, VI, VL, VK, VM, VF, VP, VS, VT, VW, VY, and VV.

When the first and second amino acid residues to be mutated are H94 and F100, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, DA, DR, DN, DD, DC, DQ, DE, DG, DH, DI, DL, DK, DM, DP, DS, DT, DW, DY, DV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are H94 and D101, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and VV.

When the first and second amino acid residues to be mutated are H94 and F102, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, DA, DR, DN, DD, DC, DQ, DE, DG, DH, DI, DL, DK, DM, DP, DS, DT, DW, DY, DV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are M95 and Y96, the double point mutants envisioned are as follows:

AA, AR, AN, AD, AC, AQ, AE, AG, AH, AI, AL, AK, AM, AF, AP, AS, AT, AW, AV, RA, RR, RN, RD, RC, RQ, RE, RG, RH, RI, RL, RK, RM, RF, RP, RS, RT, RW, RV, NA, NR, NN, ND, NC, NQ, NE, NG, NH, NI, NL, NK, NM, NF, NP, NS, NT, NW, NV, DA, DR, DN, DD, DC, DQ, DE, DG, DH, DI, DL, DK, DM, DF, DP, DS, DT, DW, DV, CA, CR, CN, CD, CC, CQ, CE, CG, CH, CI, CL, CK, CM, CF, CP, CS, CT, CW, CV, QA, QR, QN, QD, QC, QQ, QE, QG, QH, QI, QL, QK, QM, QF, QP, QS, QT, QW, QV, EA, ER, EN, ED, EC, EQ, EE, EG, EH, EI, EL, EK, EM, EF, EP, ES, ET, EW, EV, GA, GR, GN, GD, GC, GQ, GE, GG, GH, GI, GL, GK, GM, GF, GP, GS, GT, GW, GV, HA, HR, HN, HD, HC, HQ, HE, HG, HH, HI, HL, HK, HM, HF, HP, HS, HT, HW, HV, IA, IR, IN, ID, IC, IQ, IE, IG, IH, II, IL, IK, IM, IF, IP, IS, IT, IW, IV, LA, LR, LN, LD, LC, LQ, LE, LG, LH, LI, LL, LK, LM, LF, LP, LS, LT, LW, LV, KA, KR, KN, KD, KC, KQ, KE, KG, KH, KI, KL, KK, KM, KF, KP, KS, KT, KW, KV, FA, FR, FN, FD, FC, FQ, FE, FG, FH, FI, FL, FK, FM, FF, FP, FS, FT, FW, FV, PA, PR, PN, PD, PC, PQ, PE, PG, PH, PI, PL, PK, PM, PF, PP, PS, PT, PW, PV, SA, SR, SN, SD, SC, SQ, SE, SG, SH, SI, SL, SK, SM, SF, SP, SS, ST, SW, SV, TA, TR, TN, TD, TC, TQ, TE, TG, TH, TI, TL, TK, TM, TF, TP, TS, TT, TW, TV, WA, WR, WN, WD, WC, WQ, WE, WG, WH, WI, WL, WK, WM, WF, WP, WS, WT, WW, WV, YA, YR, YN, YD, YC, YQ, YE, YG, YH, YI, YL, YK, YM, YF, YP, YS, YT, YW, YV, VA, VR, VN, VD, VC, VQ, VE, VG, VH, VI, VL, VK, VM, VF, VP, VS, VT, VW, and W.

When the first and second amino acid residues to be mutated are M95 and D97, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and W.

When the first and second amino acid residues to be mutated are M95 and D98, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and W.

When the first and second amino acid residues to be mutated are M95 and G99, the double point mutants envisioned are as follows:

AA, AR, AN, AD, AC, AQ, AE, AH, AI, AL, AK, AM, AF, AP, AS, AT, AW, AY, AV, RA, RR, RN, RD, RC, RQ, RE, RH, RI, RL, RK, RM, RF, RP, RS, RT, RW, RY, RV, NA, NR, NN, ND, NC, NQ, NE, NH, NI, NL, NK, NM, NF, NP, NS, NT, NW, NY, NV, DA, DR, DN, DD, DC, DQ, DE, DH, DI, DL, DK, DM, DF, DP, DS, DT, DW, DY, DV, CA, CR, CN, CD, CC, CQ, CE, CH, CI, CL, CK, CM, CF, CP, CS, CT, CW, CY, CV, QA, QR, QN, QD, QC, QQ, QE, QH, QI, QL, QK, QM, QF, QP, QS, QT, QW, QY, QV, EA, ER, EN, ED, EC, EQ, EE, EH, EI, EL, EK, EM, EF, EP, ES, ET, EW, EY, EV, GA, GR, GN, GD, GC, GQ, GE, GH, GI, GL, GK, GM, GF, GP, GS, GT, GW, GY, GV, HA, HR, HN, HD, HC, HQ, HE, HH, HI, HL, HK, HM, HF, HP, HS, HT, HW, HY, HV, IA, IR, IN, ID, IC, IQ, IE, IH, II, IL, IK, IM, IF, IP, IS, IT, IW, IY, IV, LA, LR, LN, LD, LC, LQ, LE, LH, LI, LL, LK, LM, LF, LP, LS, LT, LW, LY, LV, KA, KR, KN, KD, KC, KQ, KE, KH, KI, KL, KK, KM, KF, KP, KS, KT, KW, KY, KV, FA, FR, FN, FD, FC, FQ, FE, FH, FI, FL, FK, FM, FF, FP, FS, FT, FW, FY, FV, PA, PR, PN, PD, PC, PQ, PE, PH, PI, PL, PK, PM, PF, PP, PS, PT, PW, PY, PV, SA, SR, SN, SD, SC, SQ, SE, SH, SI, SL, SK, SM, SF, SP, SS, ST, SW, SY, SV, TA, TR, TN, TD, TC, TQ, TE, TH, TI, TL, TK, TM, TF, TP, TS, TT, TW, TY, TV, WA, WR, WN, WD, WC, WQ, WE, WH, WI, WL, WK, WM, WF, WP, WS, WT, WW, WY, WV, YA, YR, YN, YD, YC, YQ, YE, YH, YI, YL, YK, YM, YF, YP, YS, YT, YW, YY, YV, VA, VR, VN, VD, VC, VQ, VE, VH, VI, VL, VK, VM, VF, VP, VS, VT, VW, VY, and W.

When the first and second amino acid residues to be mutated are M95 and F100, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, DA, DR, DN, DD, DC, DQ, DE, DG, DH, DI, DL, DK, DM, DP, DS, DT, DW, DY, DV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are M95 and D101, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and W.

When the first and second amino acid residues to be mutated are M95 and F102, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, DA, DR, DN, DD, DC, DQ, DE, DG, DH, DI, DL, DK, DM, DP, DS, DT, DW, DY, DV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are Y96 and D97, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, VA, VR, VN, VC, VQ, VE, VG, VH, VI, VL, VK, VM, VF, VP, VS, VT, VW, VY, and W.

When the first and second amino acid residues to be mutated are Y96 and D98, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, VA, VR, VN, VC, VQ, VE, VG, VH, VI, VL, VK, VM, VF, VP, VS, VT, VW, VY, and W.

When the first and second amino acid residues to be mutated are Y96 and G99, the double point mutants envisioned are as follows:

AA, AR, AN, AD, AC, AQ, AE, AH, AI, AL, AK, AM, AF, AP, AS, AT, AW, AY, AV, RA, RR, RN, RD, RC, RQ, RE, RH, RI, RL, RK, RM, RF, RP, RS, RT, RW, RY, RV, NA, NR, NN, ND, NC, NQ, NE, NH, NI, NL, NK, NM, NF, NP, NS, NT, NW, NY, NV, DA, DR, DN, DD, DC, DQ, DE, DH, DI, DL, DK, DM, DF, DP, DS, DT, DW, DY, DV, CA, CR, CN, CD, CC, CQ, CE, CH, CI, CL, CK, CM, CF, CP, CS, CT, CW, CY, CV, QA, QR, QN, QD, QC, QQ, QE, QH, QI, QL, QK, QM, QF, QP, QS, QT, QW, QY, QV, EA, ER, EN, ED, EC, EQ, EE, EH, EI, EL, EK, EM, EF, EP, ES, ET, EW, EY, EV, GA, GR, GN, GD, GC, GQ, GE, GH, GI, GL, GK, GM, GF, GP, GS, GT, GW, GY, GV, HA, HR, HN, HD, HC, HQ, HE, HH, HI, HL, HK, HM, HF, HP, HS, HT, HW, HY, HV, IA, IR, IN, ID, IC, IQ, IE, IH, II, IL, IK, IM, IF, IP, IS, IT, IW, IY, IV, LA, LR, LN, LD, LC, LQ, LE, LH, LI, LL, LK, LM, LF, LP, LS, LT, LW, LY, LV, KA, KR, KN, KD, KC, KQ, KE, KH, KI, KL, KK, KM, KF, KP, KS, KT, KW, KY, KV, MA, MR, MN, MD, MC, MQ, ME, MH, MI, ML, MK, MM, MF, MP, MS, MT, MW, MY, MV, FA, FR, FN, FD, FC, FQ, FE, FH, FI, FL, FK, FM, FF, FP, FS, FT, FW, FY, FV, PA, PR, PN, PD, PC, PQ, PE, PH, PI, PL, PK, PM, PF, PP, PS, PT, PW, PY, PV, SA, SR, SN, SD, SC, SQ, SE, SH, SI, SL, SK, SM, SF, SP, SS, ST, SW, SY, SV, TA, TR, TN, TD, TC, TQ, TE, TH, TI, TL, TK, TM, TF, TP, TS, TT, TW, TY, TV, WA, WR, WN, WD, WC, WQ, WE, WH, WI, WL, WK, WM, WF, WP, WS, WT, WW, WY, WV, VA, VR, VN, VD, VC, VQ, VE, VH, VI, VL, VK, VM, VF, VP, VS, VT, VW, VY, and W.

When the first and second amino acid residues to be mutated are Y96 and F100, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, DA, DR, DN, DD, DC, DQ, DE, DG, DH, DI, DL, DK, DM, DP, DS, DT, DW, DY, DV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, VA, VR, VN, VD, VC, VQ, VE, VG, VH, VI, VL, VK, VM, VP, VS, VT, VW, VY, and W.

When the first and second amino acid residues to be mutated are Y96 and D101, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, VA, VR, VN, VC, VQ, VE, VG, VH, VI, VL, VK, VM, VF, VP, VS, VT, VW, VY, and W.

When the first and second amino acid residues to be mutated are Y96 and F102, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, DA, DR, DN, DD, DC, DQ, DE, DG, DH, DI, DL, DK, DM, DP, DS, DT, DW, DY, DV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, VA, VR, VN, VD, VC, VQ, VE, VG, VH, VI, VL, VK, VM, VP, VS, VT, VW, VY, and W.

When the first and second amino acid residues to be mutated are D97 and D98, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and W.

When the first and second amino acid residues to be mutated are D97 and G99, the double point mutants envisioned are as follows:

AA, AR, AN, AD, AC, AQ, AE, AH, AI, AL, AK, AM, AF, AP, AS, AT, AW, AY, AV, RA, RR, RN, RD, RC, RQ, RE, RH, RI, RL, RK, RM, RF, RP, RS, RT, RW, RY, RV, NA, NR, NN, ND, NC, NQ, NE, NH, NI, NL, NK, NM, NF, NP, NS, NT, NW, NY, NV, CA, CR, CN, CD, CC, CQ, CE, CH, CI, CL, CK, CM, CF, CP, CS, CT, CW, CY, CV, QA, QR, QN, QD, QC, QQ, QE, QH, QI, QL, QK, QM, QF, QP, QS, QT, QW, QY, QV, EA, ER, EN, ED, EC, EQ, EE, EH, EI, EL, EK, EM, EF, EP, ES, ET, EW, EY, EV, GA, GR, GN, GD, GC, GQ, GE, GH, GI, GL, GK, GM, GF, GP, GS, GT, GW, GY, GV, HA, HR, HN, HD, HC, HQ, HE, HH, HI, HL, HK, HM, HF, HP, HS, HT, HW, HY, HV, IA, IR, IN, ID, IC, IQ, IE, IH, II, IL, IK, IM, IF, IP, IS, IT, IW, IY, IV, LA, LR, LN, LD, LC, LQ, LE, LH, LI, LL, LK, LM, LF, LP, LS, LT, LW, LY, LV, KA, KR, KN, KD, KC, KQ, KE, KH, KI, KL, KK, KM, KF, KP, KS, KT, KW, KY, KV, MA, MR, MN, MD, MC, MQ, ME, MH, MI, ML, MK, MM, MF, MP, MS, MT, MW, MY, MV, FA, FR, FN, FD, FC, FQ, FE, FH, FI, FL, FK, FM, FF, FP, FS, FT, FW, FY, FV, PA, PR, PN, PD, PC, PQ, PE, PH, PI, PL, PK, PM, PF, PP, PS, PT, PW, PY, PV, SA, SR, SN, SD, SC, SQ, SE, SH, SI, SL, SK, SM, SF, SP, SS, ST, SW, SY, SV, TA, TR, TN, TD, TC, TQ, TE, TH, TI, TL, TK, TM, TF, TP, TS, TT, TW, TY, TV, WA, WR, WN, WD, WC, WQ, WE, WH, WI, WL, WK, WM, WF, WP, WS, WT, WW, WY, WV, YA, YR, YN, YD, YC, YQ, YE, YH, YI, YL, YK, YM, YF, YP, YS, YT, YW, YY, YV, VA, VR, VN, VD, VC, VQ, VE, VH, VI, VL, VK, VM, VF, VP, VS, VT, VW, VY, and W.

When the first and second amino acid residues to be mutated are D97 and F100, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are D97 and D101, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and W.

When the first and second amino acid residues to be mutated are D97 and F102, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are D98 and G99, the double point mutants envisioned are as follows:

AA, AR, AN, AD, AC, AQ, AE, AH, AI, AL, AK, AM, AF, AP, AS, AT, AW, AY, AV, RA, RR, RN, RD, RC, RQ, RE, RH, RI, RL, RK, RM, RF, RP, RS, RT, RW, RY, RV, NA, NR, NN, ND, NC, NQ, NE, NH, NI, NL, NK, NM, NF, NP, NS, NT, NW, NY, NV, CA, CR, CN, CD, CC, CQ, CE, CH, CI, CL, CK, CM, CF, CP, CS, CT, CW, CY, CV, QA, QR, QN, QD, QC, QQ, QE, QH, QI, QL, QK, QM, QF, QP, QS, QT, QW, QY, QV, EA, ER, EN, ED, EC, EQ, EE, EH, EI, EL, EK, EM, EF, EP, ES, ET, EW, EY, EV, GA, GR, GN, GD, GC, GQ, GE, GH, GI, GL, GK, GM, GF, GP, GS, GT, GW, GY, GV, HA, HR, HN, HD, HC, HQ, HE, HH, HI, HL, HK, HM, HF, HP, HS, HT, HW, HY, HV, IA, IR, IN, ID, IC, IQ, IE, IH, II, IL, IK, IM, IF, IP, IS, IT, IW, IY, IV, LA, LR, LN, LD, LC, LQ, LE, LH, LI, LL, LK, LM, LF, LP, LS, LT, LW, LY, LV, KA, KR, KN, KD, KC, KQ, KE, KH, KI, KL, KK, KM, KF, KP, KS, KT, KW, KY, KV, MA, MR, MN, MD, MC, MQ, ME, MH, MI, ML, MK, MM, MF, MP, MS, MT, MW, MY, MV, FA, FR, FN, FD, FC, FQ, FE, FH, FI, FL, FK, FM, FF, FP, FS, FT, FW, FY, FV, PA, PR, PN, PD, PC, PQ, PE, PH, PI, PL, PK, PM, PF, PP, PS, PT, PW, PY, PV, SA, SR, SN, SD, SC, SQ, SE, SH, SI, SL, SK, SM, SF, SP, SS, ST, SW, SY, SV, TA, TR, TN, TD, TC, TQ, TE, TH, TI, TL, TK, TM, TF, TP, TS, TT, TW, TY, TV, WA, WR, WN, WD, WC, WQ, WE, WH, WI, WL, WK, WM, WF, WP, WS, WT, WW, WY, WV, YA, YR, YN, YD, YC, YQ, YE, YH, YI, YL, YK, YM, YF, YP, YS, YT, YW, YY, YV, VA, VR, VN, VD, VC, VQ, VE, VH, VI, VL, VK, VM, VF, VP, VS, VT, VW, VY, and W.

When the first and second amino acid residues to be mutated are D98 and F100, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are D98 and D101, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and W.

When the first and second amino acid residues to be mutated are D98 and F102, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are G99 and F100, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, DA, DR, DN, DD, DC, DQ, DE, DG, DH, DI, DL, DK, DM, DP, DS, DT, DW, DY, DV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are G99 and D101, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, FA, FR, FN, FC, FQ, FE, FG, FH, FI, FL, FK, FM, FF, FP, FS, FT, FW, FY, FV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and W.

When the first and second amino acid residues to be mutated are G99 and F102, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, DA, DR, DN, DD, DC, DQ, DE, DG, DH, DI, DL, DK, DM, DP, DS, DT, DW, DY, DV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are F100 and D101, the double point mutants envisioned are as follows:

AA, AR, AN, AC, AQ, AE, AG, AH, AI, AL, AK, AM, AF, AP, AS, AT, AW, AY, AV, RA, RR, RN, RC, RQ, RE, RG, RH, RI, RL, RK, RM, RF, RP, RS, RT, RW, RY, RV, NA, NR, NN, NC, NQ, NE, NG, NH, NI, NL, NK, NM, NF, NP, NS, NT, NW, NY, NV, DA, DR, DN, DC, DQ, DE, DG, DH, DI, DL, DK, DM, DF, DP, DS, DT, DW, DY, DV, CA, CR, CN, CC, CQ, CE, CG, CH, CI, CL, CK, CM, CF, CP, CS, CT, CW, CY, CV, QA, QR, QN, QC, QQ, QE, QG, QH, QI, QL, QK, QM, QF, QP, QS, QT, QW, QY, QV, EA, ER, EN, EC, EQ, EE, EG, EH, EI, EL, EK, EM, EF, EP, ES, ET, EW, EY, EV, GA, GR, GN, GC, GQ, GE, GG, GH, GI, GL, GK, GM, GF, GP, GS, GT, GW, GY, GV, HA, HR, HN, HC, HQ, HE, HG, HH, HI, HL, HK, HM, HF, HP, HS, HT, HW, HY, HV, IA, IR, IN, IC, IQ, IE, IG, IH, II, IL, IK, IM, IF, IP, IS, IT, IW, IY, IV, LA, LR, LN, LC, LQ, LE, LG, LH, LI, LL, LK, LM, LF, LP, LS, LT, LW, LY, LV, KA, KR, KN, KC, KQ, KE, KG, KH, KI, KL, KK, KM, KF, KP, KS, KT, KW, KY, KV, MA, MR, MN, MC, MQ, ME, MG, MH, MI, ML, MK, MM, MF, MP, MS, MT, MW, MY, MV, PA, PR, PN, PC, PQ, PE, PG, PH, PI, PL, PK, PM, PF, PP, PS, PT, PW, PY, PV, SA, SR, SN, SC, SQ, SE, SG, SH, SI, SL, SK, SM, SF, SP, SS, ST, SW, SY, SV, TA, TR, TN, TC, TQ, TE, TG, TH, TI, TL, TK, TM, TF, TP, TS, TT, TW, TY, TV, WA, WR, WN, WC, WQ, WE, WG, WH, WI, WL, WK, WM, WF, WP, WS, WT, WW, WY, WV, YA, YR, YN, YC, YQ, YE, YG, YH, YI, YL, YK, YM, YF, YP, YS, YT, YW, YY, YV, VA, VR, VN, VC, VQ, VE, VG, VH, VI, VL, VK, VM, VF, VP, VS, VT, VW, VY, and W.

When the first and second amino acid residues to be mutated are F100 and F102, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, DA, DR, DN, DD, DC, DQ, DE, DG, DH, DI, DL, DK, DM, DP, DS, DT, DW, DY, DV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

When the first and second amino acid residues to be mutated are D101 and F102, the double point mutants envisioned are as follows:

AA, AR, AN, AD, AC, AQ, AE, AG, AH, AI, AL, AK, AM, AP, AS, AT, AW, AY, AV, RA, RR, RN, RD, RC, RQ, RE, RG, RH, RI, RL, RK, RM, RP, RS, RT, RW, RY, RV, NA, NR, NN, ND, NC, NQ, NE, NG, NH, NI, NL, NK, NM, NP, NS, NT, NW, NY, NV, CA, CR, CN, CD, CC, CQ, CE, CG, CH, CI, CL, CK, CM, CP, CS, CT, CW, CY, CV, QA, QR, QN, QD, QC, QQ, QE, QG, QH, QI, QL, QK, QM, QP, QS, QT, QW, QY, QV, EA, ER, EN, ED, EC, EQ, EE, EG, EH, EI, EL, EK, EM, EP, ES, ET, EW, EY, EV, GA, GR, GN, GD, GC, GQ, GE, GG, GH, GI, GL, GK, GM, GP, GS, GT, GW, GY, GV, HA, HR, HN, HD, HC, HQ, HE, HG, HH, HI, HL, HK, HM, HP, HS, HT, HW, HY, HV, IA, IR, IN, ID, IC, IQ, IE, IG, IH, II, IL, IK, IM, IP, IS, IT, IW, IY, IV, LA, LR, LN, LD, LC, LQ, LE, LG, LH, LI, LL, LK, LM, LP, LS, LT, LW, LY, LV, KA, KR, KN, KD, KC, KQ, KE, KG, KH, KI, KL, KK, KM, KP, KS, KT, KW, KY, KV, MA, MR, MN, MD, MC, MQ, ME, MG, MH, MI, ML, MK, MM, MP, MS, MT, MW, MY, MV, FA, FR, FN, FD, FC, FQ, FE, FG, FH, FI, FL, FK, FM, FP, FS, FT, FW, FY, FV, PA, PR, PN, PD, PC, PQ, PE, PG, PH, PI, PL, PK, PM, PP, PS, PT, PW, PY, PV, SA, SR, SN, SD, SC, SQ, SE, SG, SH, SI, SL, SK, SM, SP, SS, ST, SW, SY, SV, TA, TR, TN, TD, TC, TQ, TE, TG, TH, TI, TL, TK, TM, TP, TS, TT, TW, TY, TV, WA, WR, WN, WD, WC, WQ, WE, WG, WH, WI, WL, WK, WM, WP, WS, WT, WW, WY, WV, YA, YR, YN, YD, YC, YQ, YE, YG, YH, YI, YL, YK, YM, YP, YS, YT, YW, YY, YV, VA, VR, VN, VD, VC, VQ, VE, VG, VH, VI, VL, VK, VM, VP, VS, VT, VW, VY, and W.

### REFERENCES

P. Maddon, et al., U.S. Pat. No. 6,451,313.
W. Dall'Acqua, et al., U.S. Pat. No. 7,083,784.
W. Olson, et al., U.S. Pat. No. 7,122,185.
L. Presta, et al., U.S. Pat. No. 7,332,581.
V. M. Litwin, et al., U.S. Pat. No. 7,345,153.
R. S. McIvor, et al., U.S. Pat. No. 9,827,295.
P. Hotez, et al., U.S. Patent Application No. 20160376321.
D. Ballon, et al., U.S. Patent Publication No. 20170067028.
G. Buchliss, et al., U.S. Patent Publication No. 20190038724.
J. Zhou, et al., U.S. Patent Publication No. 20190078099.
M. Gasmi, et al., U.S. Patent Publication No. 20190160187.
J. A. Bluestone, et al., International Publication No. WO/1994/028027.
S. A. Morgan, et al., International Publication No. WO/1994/029351.
R. J. Owens, et al., International Publication No. WO/1995/026403.
P. J. Carter, et al., International Publication No. WO/1996/027011.
G. A. Lazar, et al., International Publication No. WO/2004/029207.
R. P. Rother, et al., International Publication No. WO/2005/007809.
A. Chamberlain, et al., International Publication No. WO/2009/086320.
T. A. Stadheim, et al., International Publication No. WO/2011/149999.
H. Zhou, International Publication No. WO/2017/079369.
Adeno-Associated Virus (AAV) Guide, Addgene Catalog (https://www.addgene. org/viral-vectors/aav/aav-guide/).
Amicus, Thermo Fisher's Brammer Bio Partner on Gene Therapy Manufacturing, Genetic Engineering & Biotechnology News, Jul. 2, 2019.
T. M. Antalis, et al., Membrane-anchored serine proteases in health and disease, Progress in Molecular Biology and Translational Science, Vol. 99 (2011).
M. Bolles, et al., A double inactivated severe acute respiratory syndrome coronavirus vaccine provides incomplete protection in mice and induces increased eosinophilic proinflammatory pulmonary response upon challenge, J. of Virology, December 2011, 12201-12215.
E. M. Bouricha, et al., In silico analysis of ACE2 orthologues to predict animal host range with high susceptibility to SARS-CoV-2, 3 Biotech, 10, Article number: 483 (2020).
P. Breining, et al., Camostat mesylate against SARS-CoV-2 and COVID-19-Rationale, dosing and safety, Basic and Clinical Pharmacology & Toxicology, Vol. 128, Issue 2, February 2021, Pages 204-212.
D. A. Brindley, et al., Emerging Platform Bioprocesses for Viral Vectors and Gene Therapies, Bioprocess International, Apr. 18, 2016.
U. Brinkmann and R. E. Kontermann, The making of bispecific antibodies, mAbs, Vol. 9, 2:182-212 (2017).
T. H. Bugge, et al., Type II transmembrane serine proteases, J. Biol. Chem., 284(35): 23177-23181 (2009).
D. R. Burton and L. M. Walker, Rational Vaccine Design in the Time of COVID-19, Cell Host & Microbe, 27:695-698, May 13, 2020.
E. Callaway, The Race for Coronavirus Vaccines, Nature 580:576-77 (Apr. 30, 2020).
J. R. Cantor, et al., Therapeutic enzyme deimmunization by combinatorial T-cell epitope removal using neutral drift, Proc Natl Acad Sci USA, 2011 Jan. 25; 108(4): 1272-1277.
W. H. Chen, et al., The SARS-CoV-2 Vaccine Pipeline: an Overview, Curr. Tropical Med. Reports, Springer Nature Switzerland AG (2020).
J. R. Chevillet, et al., Identification and characterization of small-molecule inhibitors of hepsin, Mol. Cancer Ther. 2008 October; 7(10): 3343-3351.
F. Chiappelli, 2019-nCoV—Toward a 4^{th} Generation Vaccine, Bioinformation 16(2):139-144 (2020).
R. V. Chikhale, et al., Identification of potential anti-TMPRSS2 natural products through homology modelling, virtual screening and molecular dynamics simulation studies, J. of Biomolecular Structure and Dynamics, Aug. 3, 2020 (https://doi.org/10.1080/07391102.2020.1798813).
M. L. Chiu and G. L. Gilliland, Engineering antibody therapeutics, Current Opinion in Structural Biology 2016, 38:163-173.
S. Y. Choi, et al., Type II transmembrane serine proteases in cancer and viral infections, Trends in Mol. Med. 15(7): 303-312 (2009).
T.-W. Chun, et al., Durable Control of HIV Infection in the Absence of Antiretroviral Therapy: Opportunities and Obstacles, JAMA. 2019; 322(1): 27-28.
N. E. Clarke and A. J. Turner, Angiotensin-Converting Enzyme 2: The First Decade, Intl. J. of Hypertension, Volume 2012, Article ID 307315, pp. 1-12.
D. Clayton, et al., Structural determinants for binding to angiotensin converting enzyme 2 (ACE2) and angiotensin receptors 1 and 2, Front. Pharmacol., 30 Jan. 2015.
C. M. Coleman, et al., Purified coronavirus spike protein nanoparticles induce coronavirus neutralizing antibodies in mice, Vaccine 32 (2014) 3169-3174.
B. Coutard, et al., The spike glycoprotein of the new coronavirus 2019-nCoV contains a furin-like cleavage site absent in CoV of the same clade, Antiviral Research 176 (2020) 104742.
M. C. Crank, et al., A proof of concept for structure-based vaccine design targeting RSV in humans, Science 365, 505-509 (2019).
S. Daya and K. I. Berns, Gene Therapy Using Adeno-Associated Virus Vectors, Clinical Microbiology Reviews, October 2008, Vol. 21, No. 4, p. 583-593.
C. E. Deal and A. B. Balazs, Vectored Antibody Gene Delivery for the Prevention or Treatment of HIV Infection, Curr Opin HIV AIDS. 2015 May; 10(3): 190-197.
M. S. Diamond and T. C. Pierson, The Challenges of Vaccine Development against a New Virus during a Pandemic, Cell Host & Microbe, 27, May 13, 2020.
M. Donoghue, et al., A Novel Angiotensin-Converting Enzyme-Related Carboxypeptidase (ACE2) Converts Angiotensin I to Angiotensin 1-9, Circulation Res., Sep. 1, 2000.
L. M. Drouin and M. Agbandje-McKenna, Adeno-associated virus structural biology as a tool in vector development, Future Virol. 2013 December; 8(12): 1183-1199.
Y. Du, et al., A broadly neutralizing humanized ACE2-targeting antibody against SARS-CoV-2 variants. Nat Commun 12, 5000 (2021) (https://doi.org/10.1038/s41467-021-25331-x), including Supplemental Information (https://static-content.springer.com/esm/art %3A10.1038%2Fs41467-021-25331-x/Media Objects/41467_2021_25331_MOESM1_ESM.pdf).
C. Dumet, et al., Insights into the IgG heavy chain engineering patent landscape as applied to IgG4 antibody development, mAbs, Vol. 11, 8:1341-1350 (2019).
M. Ferarri, et al., Characterization of a novel ACE2-based therapeutic with enhanced rather than reduced activity against SARS-CoV-2 variants, J. of Virology, vol. 95, issue 19, October 2021.
S. P. Fuchs, et al., Recombinant AAV Vectors for Enhanced Expression of Authentic IgG, PLOS ONE|DOI:10.1371/journal.pone.0158009, pp. 1-19, Jun. 22, 2016.
S. P. Fuchs, et al., Liver-directed but not muscle-directed AAV-antibody gene transfer limits humoral immune responses in rhesus monkeys, Mol. Therapy: Methods & Clin. Dev., 16:94-102 (March 2020).
M. R. Gardner, AAV-delivered eCD4-Ig protects rhesus macaques from high-dose SIVmac239 challenges, Sci. Transl. Med. 11, eaau5409 (Jul. 24, 2019).
M. R. Gardner, et al., Anti-Drug Antibody Responses Impair Prophylaxis Mediated by AAV-Delivered HIV-1 Broadly Neutralizing Antibodies, Molecular Therapy, Vol. 27, No. 3, 650-660 (March 2019).
M. Godar, et al., Therapeutic bispecific antibody formats: A patent applications review (1994-2017), Expert Opinion on Therapeutic Patents, Vol. 28, 3:251-276 (2018).
K. Gopinath, et al., Screening of Natural Products Targeting SARS-CoV-2-ACE2 Receptor Interface—A MixMD Based HTVS Pipeline, (2020) Front. Chem. 8:589769.
Y-R Guo, et al., The origin, transmission and clinical therapies on coronavirus disease 2019 (COVID-19) outbreak—an update on the status, Military Medical Res. (2020) 7:11.
J. L. Guy, et al., Identification of critical active-site residues in angiotensin-converting enzyme 2 (ACE2) by site-directed mutagenesis, FEBS Journal, 272 (2005) 3512-3520.
N. Halama, et al., Tumoral Immune Cell Exploitation in Colorectal Cancer Metastases Can Be Targeted Effectively by Anti-CCR5 Therapy in Cancer Patients, 2016, Cancer Cell 29, 587-601.
I. Hamming, et al., Tissue distribution of ACE2 protein, the functional receptor for SARS coronavirus. A first step in understanding SARS pathogenesis, J. of Pathology, 2004, 203:631-637.
Y. Han and P. Kral, Computational Design of ACE2-Based Peptide Inhibitors of SARS-CoV-2, ACS Nano 2020, 14, 4, 5143-5147, Apr. 14, 2020.
M. Hoffmann, et al., SARS-CoV-2 cell entry depends on ACE2 and TMPRSS2 and is blocked by a clinically proven protease inhibitor, Cell, 181:1-10 (2020).
M. Hoffmann, et al., A Multibasic Cleavage Site in the Spike Protein of SARS-CoV-2 Is Essential for Infection of Human Lung Cells, Molecular Cell, 78:1-6 (2020).
M. Hoffman, et al., SARS-CoV-2 variants B.1.351 and P.1 escape from neutralizing antibodies, Cell 11954 (2021).
K. Hollevoet and P. J. Declerck, State of play and clinical prospects of antibody gene transfer, J Transl Med (2017) 15:131.
D. Hu, et al., Effective Optimization of Antibody Affinity by Phage Display Integrated with High-Throughput DNA Synthesis and Sequencing Technologies, PLOS ONE DOI:10.1371/journal.pone.0129125 Jun. 5, 2015.
Y. Huang, et al., Structural and functional properties of SARS-CoV-2 spike protein: potential antivirus drug development for COVID-19, Acta Pharmacologica Sinica, volume 41, pages 1141-1149 (2020).
Human Monoclonal Antibodies for Human ACE2, Twist Biopharma (2020).
C. J. Hutchings, A review of antibody-based therapeutics targeting G protein-coupled receptors: an update, Expert Opinion on Biological Therapy, 1744-7682 (online) (Apr. 8, 2020).
C. Jackson, et al., Mechanism of SARS-CoV-2 entry into cells, Nature Reviews, Oct. 5, 2021.
R. Jefferys, HIV vaccine update: the “Miami macaque” as proof of-concept breakthrough? i-base, Jan. 22, 2018. (http://i-base.info/htb/date/2018/01/22).
G. U. Jeong, et al., Therapeutic Strategies Against COVID-19 and Structural Characterization of SARS-CoV-2: A Review, Front. Microbiol., 14 Jul. 2020.
S. Jiang, et al., SARS Vaccine Development, Emerging Infectious Diseases, 11(7): 1016-1020 (2005).
S. Jiang, et al., Roadmap to developing a recombinant coronavirus S protein receptor-binding domain vaccine for severe acute respiratory syndrome, Expert Review of Vaccines, 11(12); 1405-1413 (2012).
S. Jiang, et al., An emerging coronavirus causing pneumonia outbreak in Wuhan, China: calling for developing therapeutic and prophylactic strategies, Emerging Microbes & Infections, 9:275-277 (2020).
B. Ju, et al., Potent human neutralizing antibodies elicited by SARS-CoV-2 infection, bioRxiv doi: https://doi.org/10.1101/2020.03.21.990770.
J. Kaiser, Boys with a rare muscle disease are breathing on their own, thanks to gene therapy, May 2, 2019, Science.
Y. Kazama, et al., Hepsin, a putative membrane-associated serine protease, activates human factor VII and initiates a pathway of blood coagulation on the cell surface leading to thrombin formation, J. Biol. Chem., 1995, 270(1): 66-72.
A. Keener, The genetic shortcut to antibody drugs, Nature 564, S16-S17 (2018).
B. Kelley, Developing therapeutic monoclonal antibodies at pandemic pace, Nature Biotechnology, Apr. 21, 2020, doi: https://www.nature.com/articles/s41587-020-0512-5.
T. Kitazawa, et al., A bispecific antibody to factors IXa and X restores factor VIII hemostatic activity in a hemophilia A model, Nature Medicine, Vol. 18, No. 10, 1570-1574 (October 2012).
P.-A. Koenig, et al., Structure-guided multivalent nanobodies block SARS-CoV-2 infection and suppress mutational escape, Science 12, February 2021: Vol. 371, Issue 6530, eabe6230.
G. Kohler and C. Milstein, Continuous cultures of fused cells secreting antibody of predefined specificity, Nature 1975, 256:495-497.
T. Koschubs, et al., Allosteric antibody inhibition of human hepsin protease, Biochem J. (2012) 442:483-494.
M. A. Kotterman and D. V. Schaffer, Engineering adeno-associated viruses for clinical gene therapy, Nature Reviews Genetics|AOP, published online 20 May 2014; doi:10.1038/nrg3742.
B. Lafleur, et al., Production of human or humanized antibodies in mice, Methods Mol. Biol. 2012, 901:149-159.
C. S. Lee, et al., Adenovirus-mediated gene delivery: Potential applications for gene and cell-based therapies in the new era of personalized medicine, Genes & Diseases (2017) 4, 43-63.
R. A. Liberatore and D. D. Ho, The Miami Monkey: A Sunny Alternative to the Berlin Patient, Immunity Previews, Volume 50, Issue 3, P537-539, Mar. 19, 2019.
C. Li and RJ Samulski, Engineering adeno-associated virus vectors for gene therapy, Nature Reviews, 21:255-272 (April 2020).
F. Li, et al., Structure of SARS coronavirus spike receptor-binding domain complexed with receptor, Science, 309:1864-1868 (2005).
F. Li, Receptor recognition and cross-species infections of SARS coronavirus, Antiviral Res., October 2013, 100(1).
W. Li, et al., Receptor and viral determinants of SARS-coronavirus adaptation to human ACE2, The EMBO J., (2005) 24:1634-1643.
C. C. Lim, et al., Cognizance of Molecular Methods for the Generation of Mutagenic Phage Display Antibody Libraries for Affinity Maturation, Int. J. Mol. Sci., 2019 April; 20(8): 1861.
J. Luan, et al., Spike protein recognition of mammalian ACE2 predicts the host range and an optimized ACE2 for SARS-CoV-2 infection, Vol. 526, Issue 1, May 21, 2020, pp. 165-169.
N. Lurie, et al., Developing Covid-19 Vaccines at Pandemic Speed, N. Engl. J. Med., Perspective (April 2020).
S. Lytras, et al., The animal origin of SARS-CoV-2, Science, 10.1126/science. abh0117 (2021).
J. Ma, et al., In vitro and in vivo efficacy of a Rift valley fever virus vaccine based on pseudovirus, Hum. Vaccin. Immunother. 2019; 15(10):2286-2294.
J. M. Martinez-Navio, et al., Adeno-Associated Virus Delivery of Anti-HIV Monoclonal Antibodies Can Drive Long-Term Virologic Suppression, Immunity, 50:567-575 (2019).
J. M. Martinez-Navio, et al., Long-Term Delivery of an Anti-SIV Monoclonal Antibody With AAV, Frontiers in Immunology, March 2020, Vol. 11, Article 449.
S. Matsuyama, et al., Enhanced isolation of SARS-CoV-2 by TMPRSS2-expressing cells, PNAS, Mar. 31, 2020 117(13): 7001-7003.
K. McKeage, Ravulizumab: First Global Approval, Drugs (2019), 79:347-52.
A. D. Melin, et al., Comparative ACE2 variation and primate COVID-19 risk, Communications Biology, Volume 3, Article number 641 (2020).
T. Meng, et al., The insert sequence in SARS-CoV-2 enhances spike protein cleavage by TMPRSS, bioRxiv doi: https://www.biorxiv.org/content/10.1101/2020.02.08.926006v3.
J. K. Millet and G. R. Whittaker, Host cell proteases: critical determinants of coronavirus tropism and pathogenesis, Virus Res. 202 (2015) 120-134.
C. Mueller, et al., (2012). Production and discovery of novel recombinant adeno-associated viral vectors. Curr. Protoc. Microbiol. Chapter 14, Unit 14D.1.
S. Nagataa and I. Pastanb, Removal of B cell epitopes as a practical approach for reducing the immunogenicity of foreign protein-based therapeutics, Adv Drug Deliv Rev. 2009 Sep. 30; 61(11): 977-985.
M. F. Naso, Adeno-Associated Virus (AAV) as a Vector for Gene Therapy, BioDrugs (2017) 31:317-334.
J. Nie, et al., Establishment and validation of a pseudovirus neutralization assay for SARS-CoV-2, Emerg. Microbes Infect., 2020 December; 9(1):680-686.
D. S. Ojala, et al., Adeno-Associated Virus Vectors and Neurological Gene Therapy, The Neuroscientist, Feb. 20, 2014.
T. Ou, et al., Hydroxychloroquine-mediated inhibition of SARS-CoV-2 entry is attenuated by TMPRSS2. PLoS Pathog 17(1): e1009212 (2021).
V. Padilla-Sanchez, SARS-CoV-2 Structural Analysis of Receptor Binding Domain New Variants from United Kingdom and South Africa, Research Ideas and Outcomes 7, e62936, Jan. 15, 2021.
S. K. Panda, et al., ACE-2-Derived Biomimetic Peptides for the Inhibition of Spike Protein of SARS-CoV-2, J. Proteome Res. 2021, 20, 2, 1296-1303, Jan. 20, 2021.
L. C. Paoletti and RC Kennedy, Neutralizing antibody induced in mice by novel glycoconjugates of Human Immunodeficiency Virus Type 1 gp120 and env2-3, J. of Infectious Diseases, 2002; 186:1597-1602.
A. Paoloni-Giacobino, et al., Cloning of the TEMPRSS2 gene, which encodes a novel serine protease with transmembrane, LDLRA, and SRCR domains and maps to 21q22.3, Genomics 44:309-320 (1997).
A. B. Patel and A. Verma, COVID-19 and angiotensin-converting enzyme inhibitors and angiotensin receptor blockers: What is the evidence? JAMA, Mar. 24, 2020.
Z. Payandeh, et al., Design of an engineered ACE2 as a novel therapeutic against COVID-19, Journal of Theoretical Biology, Volume 505, 21 Nov. 2020, 110425.
A. Pena, Gene Therapy for Hemophilia A, SB-525, Showing Continued Benefits in Trial Data Update, Hemophelia News Today, Jun. 26, 2019.
A. Philippidis, Virus Supply Vexes Gene Therapy Developers, CMOs, Genetic Engineering & Biotechnology News, Dec. 14, 2017.
M. Poglitsch, et al., Recombinant expression and characterization of human and murine ACE2: Species-specific activation of the alternative renin-angiotensin-system, Intl. J. of Hypertension, Volume 2012, Article ID 428950, pp. 1-8.
T. R. D. J. Radstake, et al., Formation of antibodies against infliximab and adalimumab strongly correlates with functional drug levels and clinical responses in rheumatoid arthritis, Ann Rheum Dis 2009; 68:1739-1745.
N. Raman, et al., Virtual Screening of Natural Products Against Type II Transmembrane Serine Protease (TMPRSS2), the Priming Agent of Coronavirus 2 (SARS-CoV-2), Molecules 2020, 25, 2771.
G. J. Robbie, et al., A Novel Investigational Fc-Modified Humanized Monoclonal Antibody, Motavizumab-YTE, Has an Extended Half-Life in Healthy Adults, Antimicrobial Agents and Chemotherapy, December 2013, Vol. 57, No. 12, pp. 6147-6143.
R. A. S. Santos, et al., The ACE2/Angiotensin-(1-7)/MAS Axis of the Renin-Angiotensin System: Focus on Angiotensin-(1-7), Physiol. Rev. 98:505-553 (2018).
A. Sato, “Synthetic DNA technologies enable fast and responsive SARS-CoV-2 antibody discovery and optimization”, Twist Biopharma, Jul. 7, 2020, Webinar (https://www.youtube.com/watch?v=ceHCqy8UsXU).
Z. E. Sauna, et al., Evaluating and Mitigating the Immunogenicity of Therapeutic Proteins, Trends in Biotechnology, October 2018, Vol. 36, No. 10.
T. Schlothauer, et al., Novel human IgG1 and IgG4 Fc-engineered antibodies with completely abolished immune effector functions, Protein Engineering, Design & Selection, 2016, vol. 29, no. 10, pp. 457-466.
M. Schoof, et al., An ultrapotent synthetic nanobody neutralizes SARS-CoV-2 by stabilizing inactive Spike, Science Dec. 18, 2020: Vol. 370, Issue 6523, pp. 1473-1479.
J. Shang, et al., Structural basis of receptor recognition by SARS-CoV-2, Nature, pages 1-19, Mar. 30, 2020.
L. W. Shen, et al., TMPRSS2: a potential target for treatment of influenza virus and coronavirus infections, Biochimie 142 (2017) 1-10.
D. Sheridan, et al., Design and preclinical characterization of ALXN1210: A novel anti-C5 antibody with extended duration of action, PLOS One, Apr. 12, 2018.
K. Shirato, et al., Middle East Respiratory Syndrome coronavirus infection mediated by the transmembrane serine protease TMPRSS2, J. of Virology, 87(23): 12552-12561 (December 2013).
J. H. Shrimp, et al., An Enzymatic TMPRSS2 Assay for Assessment of Clinical Candidates and Discovery of Inhibitors as Potential Treatment of COVID-19, ACS Pharmacology & Translational Science 2020 3 (5), 997-1007.
A. Shulla, et al., A transmembrane serine protease is linked to the severe acute respiratory syndrome coronavirus receptor and activates virus entry, J. of Virology, 85(2): 873-882 (January 2011).
J.-P. Silva, et al., The S228P Mutation Prevents in Vivo and in Vitro IgG4 Fab-arm Exchange as Demonstrated using a Combination of Novel Quantitative Immunoassays and Physiological Matrix Preparation, J. Biol. Chem., 2015 Feb. 27; 290(9): 5462-5469.
S. K. Singh, et al., CCR5/CCL5 axis interaction promotes migratory and invasiveness of pancreatic cancer cells, Scientific Reports, Nature, (2018) 8:1323.
P. K. Smith, et al., Measurement of protein using bicinchoninic acid, Anal. Biochem. 150:76-85 (1985).
K. Sonawane, et al., (2020), Homology Modeling and Docking Studies of TMPRSS2 with Experimentally Known Inhibitors Camostat Mesylate, Nafamostat and Bromhexine Hydrochloride to Control SARS-Coronavirus-2. ChemRxiv. Preprint. https://doi.org/10.26434/chemrxiv.12162360.v1.
P. Sullivan, FDA approves world's most expensive drug at $2.1M, The Hill, May 24, 2019.
J. Sun, et al., COVID-19: epidemiology, evolution, and cross-disciplinary perspectives, Trends in Mol. Med., 2020, doi: http://www.cell.com/trends/molecular-medicine/retrieve/pii/S1471491420300654?_returnURL=https%3A%2F%/2Flinkinghub.elsevier.com%2Fretrieve%2Fpii%2FS1471491420300654%3Fshowall%3Dtrue.
P. Supasa, et al., Reduced neutralization of SARS-CoV-2 B.1.1.7 variant by convalescent and vaccine sera, Cell 11896 (2021).
N. Suryadevara, et al., Neutralizing and protective human monoclonal antibodies recognizing the N-terminal domain of the SARS-CoV-2 spike protein, 2021, Cell, 184:1-16.
F. V. Suurs, et al., A review of bispecific antibodies and antibody constructs in oncology and clinical challenges, Pharmacology & Therapeutics 201 (2019) 103-119.
W. Tai, et al., Characterization of the receptor-binding domain (RBD) of 2019 novel coronavirus: implication for development of RBD protein as a viral attachment inhibitor and vaccine, Cellular & Mol. Immun., Mar. 19, 2020.
S. H. Tam, et al., Functional, Biophysical, and Structural Characterization of Human IgG1 and IgG4 Fc Variants with Ablated Immune Functionality, Antibodies 2017, 6, 12.
P. Tamamis and C. A. Floudas, Elucidating a Key Anti-HIV-1 and Cancer-Associated Axis: The Structure of CCL5 (Rantes) in Complex with CCR5, Scientific Reports, Nature, (2014) 4:5447.
C.-W. Tan, et al., Pan-Sarbecovirus Neutralizing Antibodies in BNT162b2-Immunized SARS-CoV-1 Survivors, N. Engl. J. Med., Aug. 18, 2021.
X. Tian, et al., Potent binding of 2019 novel coronavirus spike protein by a SARS coronavirus-specific human monoclonal antibody, Emerging Microbes & Infections, 9:382-385 (2020).
S. R. Tipnis, et al., A Human Homolog of Angiotensin-converting Enzyme, J. Biol. Chem., 2000 Oct. 27; 275(43): 33238-43.
A. J. Turner, et al., ACE2: from vasopeptidase to SARS virus receptor, Trends in Pharm. Sci, 25(6): 291-294 (2004).
M. Vaduganathan, et al., Renin-angiotensin-aldosterone system inhibitors in patients with Covid-19, N. Engl. J. Med., Special Report (April 2020).
L. Vangelista and S. Vento, The Expanding Therapeutic Perspective of CCR5 Blockade, Front Immunol. 2017; 8:1981.
C. Vickers, et al., Hydrolysis of Biological Peptides by Human Angiotensin-converting Enzyme-related Carboxypeptidase, J. Biol. Chem., 2002 Apr. 26; 277(17): 14838-43.
Viral Vectors, Gene Therapy Net (http://www.genetherapynet.com/viral-vectors.html).
A. C. Walls, et al., Structure, function and antigenicity of the SARS-CoV-2 spike glycoprotein, bioRxiv doi: https://doi.org/10.1101/2020.02.19.956581.
Y. Wan, et al., Molecular Mechanism for Antibody-Dependent Enhancement of Coronavirus Entry, J. of Virology, Vol. 94, Issue 5, e02015-19 (March 2020).
N. Wang, et al., Subunit Vaccines Against Emerging Pathogenic Human Coronaviruses, Frontiers in Microbiology, 11: 298 (2020).
M. A. Whitt, Generation of VSV pseudotypes using recombinant DeltaG-VSV for studies on virus entry, identification of entry inhibitors, and immune responses to vaccines. J. Virol. Methods. 2010; 169(2):365-374.
S. K. Wong, et al., A 193-amino acid fragment of the SARS Coronavirus S Protein efficiently binds Angiotensin-converting Enzyme 2, J. Biol. Chem., 279(5): 3197-3201 (2004).
D. Wrapp, et al., Cryo-EM structure of the 2019-nCoV spike in the prefusion conformation, Science, 367, 1260-1263 (2020).
D. Wrapp, et al., Structural Basis for Potent Neutralization of Betacoronaviruses by Single-Domain Camelid Antibodies, Cell 181:1-12 (May 28, 2020).
Y. Wu, et al., A non-competing pair of human neutralizing antibodies block COVID-19 virus binding to its receptor ACE2, Science, 10.1126/Science.abc2241 (2020).
S. Xia, et al., Fusion mechanism of 2019-nCoV and fusion inhibitors targeting HR1 domain in spike protein, Cellular & Mol. Immunol., February 2020.
C. Xu, et al., Conformational dynamics of SARS-CoV-2 trimeric spike glycoprotein in complex with receptor ACE2 revealed by cryo-EM, Science Advances Jan. 1, 2021: Vol. 7, no. 1, eabe5575.
J. A. Xuan, et al., Antibodies neutralizing hepsin protease activity do not impact cell growth but inhibit invasion of prostate and ovarian tumor cells in culture, Cancer Res. 2006, 66(7): 3611-3619.
X. Yang, et al., Comprehensive Analysis of the Therapeutic IgG4 Antibody Pembrolizumab: Hinge Modification Blocks Half Molecule Exchange In Vitro and In Vivo, J Pharm Sci, 104:4002-4014, Aug. 26, 2015, https://doi.org/10.1002/jps.24620.
R. Zang, et al., TMPRSS2 and TMPRSS4 promote SARS-CoV-2 infection of human small intestinal enterocytes, Science Immunology 13 May 2020: Vol. 5, Issue 47, eabc3582.
H. Zhang, et al., Angiotensin-converting enzyme 2 (ACE2) as a SARS-CoV-2 receptor: molecular mechanisms and potential therapeutic target, Intensive Care Medicine, 46:586-590 (2020).
J. Zhang, et al., Structure of SARS-CoV-2 spike protein, Curr. Opinion in Virology 2021, 50:173-182.
Zhou, ACE2 and TMPRSS2 are expressed on the human ocular surface, suggesting susceptibility to SARS-CoV-2 infection, bioRxiv, doi: https://www.biorxiv.org/content/10.1101/2020.05.09.086165v1.
P. Zmora, et al., TMPRSS2 isoform 1 activates respiratory viruses and is expressed in viral target cells, PLOS ONE Sep. 17, 2015.
A. Zumla, et al., Coronaviruses—drug discovery and therapeutic options, Nature Reviews: Drug Discovery, Vol. 15, May 2016, 327-347.

1. A bispecific antibody that (i) specifically binds to the extracellular portion of human angiotensin converting enzyme 2 (hACE2); (ii) specifically binds to the extracellular portion of human TMPRSS2 (hTMPRSS2); (iii) does not significantly inhibit the ability of hACE2 to cleave angiotensin II and/or a synthetic MCA-based peptide; and (iv) specifically inhibits the entry into hACE2⁺/hTMPRSS2⁺ human cells of a pseudovirus bearing SARS-CoV-2 S protein.

2. The bispecific antibody of claim 1, wherein the bispecific antibody has a low effector function.

3. The bispecific antibody of claim 1, wherein the bispecific antibody has a long serum half-life.

4. The bispecific antibody of claim 1, wherein the bispecific antibody is an IgG4 antibody.

5. The bispecific antibody of claim 1, wherein the bispecific antibody comprises a heavy chain modification that inhibits half antibody formation.

6. The bispecific antibody of claim 1, wherein the bispecific antibody is a humanized bispecific antibody.

7. The bispecific antibody of claim 1, wherein the bispecific antibody is a human bispecific antibody.

8. An isolated nucleic acid molecule encoding (a) the bispecific antibody of claim 1, if the bispecific antibody has only one chain; or (b) one or more chains of the bispecific antibody of claim 1, if the bispecific antibody has a plurality of chains.

9. A recombinant vector comprising the nucleotide sequence of the nucleic acid molecule of claim 8 operably linked to a promoter of RNA transcription.

10. A composition comprising (i) the bispecific antibody of claim 1, and (ii) a pharmaceutically acceptable carrier.

11. A method for reducing the likelihood of a human subject's becoming infected with SARS-CoV-2 comprising administering to the subject a prophylactically effective amount of the bispecific antibody of claim 1.

12. The method of claim 11, wherein the subject has been exposed to SARS-CoV-2.

13. A method for treating a human subject who is infected with SARS-CoV-2 comprising administering to the subject a therapeutically effective amount of the bispecific antibody of claim 1.

14. The method of claim 13, wherein the subject is symptomatic of a SARS-CoV-2 infection.

15. The method of claim 13, wherein the subject is asymptomatic of a SARS-CoV-2 infection.

16. A recombinant AAV vector comprising a nucleic acid sequence encoding (a) the bispecific antibody of claim 1, if the bispecific antibody has only one chain, or (b) one or more chains of the bispecific antibody of claim 1, if the bispecific antibody has a plurality of chains.

17. The recombinant AAV vector of claim 16, wherein the nucleic acid sequence encodes all chains of the bispecific antibody.

18. A recombinant AAV particle comprising the recombinant AAV vector of claim 16.

19. A composition comprising (i) a plurality of the AAV particles of claim 18 and (ii) a pharmaceutically acceptable carrier.

20. A method for reducing the likelihood of a human subject's becoming infected with SARS-CoV-2 comprising administering to the subject a prophylactically effective number of the AAV particles of claim 18.

21. The method of claim 20, wherein the subject has been exposed to SARS-CoV-2.

22. A method for treating a human subject who is infected with SARS-CoV-2 comprising administering to the subject a therapeutically effective number of the AAV particles of claim 18.

23. The method of claim 22, wherein the subject is symptomatic of a SARS-CoV-2 infection.

24. The method of claim 22, wherein the subject is asymptomatic of a SARS-CoV-2 infection.

25. A kit comprising, in separate compartments, (a) a diluent and (b) a suspension of the bispecific antibody of claim 1.

26. A kit comprising, in separate compartments, (a) a diluent and (b) the bispecific antibody of claim 1 in lyophilized form.

27. A kit comprising, in separate compartments, (a) a diluent and (b) a suspension of a plurality of the recombinant AAV particles of claim 18.


SEQUENCE LISTING (21 sequences)

SEQ ID NO: 1 - protein, 7 aa, Artificial Sequence
  Description of Artificial Sequence Synthetic peptide
YVADAPK

SEQ ID NO: 2 - protein, 18 aa, Artificial Sequence
  Description of Artificial Sequence Synthetic peptide
CAKDRGYSSSWYGGFDYW

SEQ ID NO: 3 - protein, 16 aa, Artificial Sequence
  Description of Artificial Sequence Synthetic peptide
CARHTWWKGAGFFDHW

SEQ ID NO: 4 - protein, 17 aa, Artificial Sequence
  Description of Artificial Sequence Synthetic peptide
CARGTRFLEWSLPLDVW

SEQ ID NO: 5 - protein, 11 aa, Artificial Sequence
  Description of Artificial Sequence Synthetic peptide
CATTENPNPRW

SEQ ID NO: 6 - protein, 11 aa, Artificial Sequence
  Description of Artificial Sequence Synthetic peptide
CATTEDPYPRW

SEQ ID NO: 7 - protein, 15 aa, Artificial Sequence
  Description of Artificial Sequence Synthetic peptide
CARASPNTGWHFDHW

SEQ ID NO: 8 - protein, 11 aa, Artificial Sequence
  Description of Artificial Sequence Synthetic peptide
CATTMNPNPRW

SEQ ID NO: 9 - protein, 15 aa, Artificial Sequence
  Description of Artificial Sequence Synthetic peptide
CAAIAYEEGVYRWDW

SEQ ID NO: 10 - protein, 4 aa, Artificial Sequence
  Description of Artificial Sequence Synthetic peptide
KQLR

SEQ ID NO: 11 - protein, 6 aa, Artificial Sequence
  Description of Artificial Sequence Synthetic peptide
ZQRRZK

SEQ ID NO: 12 - protein, 6 aa, Artificial Sequence
  Description of Artificial Sequence Synthetic 6xHis tag
HHHHHH

SEQ ID NO: 13 - protein, 4 aa, Artificial Sequence
  Description of Artificial Sequence Synthetic peptide
RKRR

SEQ ID NO: 14 - DNA, 3405 bp, Homo sapiens
cgcccaacccaagttcaaaggctgataagagagaaaatctcatgaggaggttttagtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactcaaaaaaaaaaaa

SEQ ID NO: 15 - protein, 805 aa, Homo sapiens [sequence omitted]

SEQ ID NO: 16 - DNA, 2479 bp, Homo sapiens
gtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcagatgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtttagcctgcggggtcaacttgaactcaagccgccagagcaggatcgtgggcggtgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtacaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaacaacaatatctggtggctgataggggatacaagctggggttctggctgtgccaaagcttacagaccaggagtgtacgggaatgtgatggtattcacggactggatttatcgacaaatgaaggcaaacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccatactgtgcaggctgcagtggctcccctgcccagcctgctctccctaaccccttgtccgcaaggggtgatggccggctggttgtgggcactggcggtcaattgtggaaggaagagggttggaggctgcccccattgagatcttcctgctgagtcctttccaggggccaattttggatgagcatggagctgtcacttctcagctgctggatgacttgagatgaaaaaggagagacatggaaagggagacagccaggtggcacctgcagcggctgccctctggggccacttggtagtgtccccagcctacttcacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgaatgccccttggtccctggcagggcgccaagtttggcaccatgtcggcctcttcaggcctgatagtcattggaaattgaggtccatgggggaaatcaaggatgctcagtttaaggtacactgtttccatgttatgtttctacacattgatggtggtgaccctgagttcaaagccatctt

SEQ ID NO: 17 - protein, 492 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQMYSSQRKSWHPVCQDDWNENYGRAACRDMGYKNNFYSSQGIVDDSGSTSFMKLNTSAGNVDIYKKLYHSDACSSKAVVSLRCLACGVNLNSSRQSRIVGGESALPGAWPWQVSLHVQNVHVCGGSIITPEWIVTAAHCVEKPLNNPWHWTAFAGILRQSFMFYGAGYQVQKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSNNNIWWLIGDTSWGSGCAKAYRPGVYGNVMVFTDWIYRQMKANG

SEQ ID NO: 18 - protein, 194 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 19 - protein, 193 aa, Severe acute respiratory syndrome-related coronavirus
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATV

SEQ ID NO: 20 - protein, 212 aa, Middle East respiratory syndrome-related coronavirus
QAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 21 - protein, 4 aa, Artificial Sequence
  Description of Artificial Sequence Synthetic peptide
VRPR